(19) United States
(12) Patent Application Publication
(10) Pub. No.: US 2007/0149526 A1
(43) Pub. Date: Jun. 28, 2007

(54) Diuretic and diuretic-like compound analogs

(75) Inventors: Daryl W. Hochman, Bahama, NC (US); John J. Partridge, Chapel Hill, NC (US)

Correspondence Address: MYERS BIGEL SIBLEY & SAJOVEC, PO BOX 37428, RALEIGH, NC, 27627, US

(73) Assignee: NeuroTherapeutics Pharma, L.L.C.

(21) Appl. No.: 11/549,274

(22) Filed: Oct. 13, 2006

Related U.S. Application Data
(60) Provisional application No. 60/727,564, filed on Oct. 17, 2005.

(51) Int. Cl. A61K 31/5377; A61K 31/4412; A61K 31/41; A61K 31/40; A61K 31/34

(52) U.S. Cl. 514/237.8; 514/471; 544/152; 514/381; 548/253; 514/346; 546/290; 514/408; 548/577

(57) ABSTRACT

The present invention provides compounds that are effective in treating central nervous system disorders and maintaining normal brain function. Methods of making and using the compounds are also provided.

### RELATED APPLICATION INFORMATION

This application claims the benefit of U.S. Provisional Application Ser. No. 60/727,564, filed Oct. 17, 2005, the disclosure of which is incorporated herein by reference in its entirety.

### FIELD OF THE INVENTION

The present invention relates to compounds that traverse the blood-brain barrier. The present invention also relates to intermediates of these compounds, pharmaceutical compositions containing these compounds, and methods of using the compounds. Such compounds are particularly useful for regulation of central nervous system disorders, and are particularly useful for maintaining and enhancing normal central nervous system function.

### BACKGROUND OF THE INVENTION

The blood-brain barrier (BBB) is a physical barrier and system of cellular transport mechanisms between the blood vessels in the central nervous system (CNS) and most areas of the CNS itself. The BBB maintains homeostasis by restricting the entry of potentially harmful chemicals from the blood, and by allowing the entry of essential nutrients, However, the BBB can pose a formidable barrier to delivery of pharmacological agents to the CNS for treatment of CNS disorders or maintaining or enhancing normal and desirable brain functions, such as cognition, learning and memory. More specific CNS disorders and functions are described below.

Epilepsy

Epilepsy is characterized by abnormal discharges of cerebral neurons and is typically manifested as various types of seizures. Epileptiform activity is identified with spontaneously occurring synchronized discharges of neuronal populations that can be measured using electrophysiological techniques. Epilepsy is one of the most common neurological disorders, affecting about 1% of the population. There are various forms of epilepsy, including idiopathic, symptomatic and cryptogenic. Genetic predisposition is thought to be the predominant etiologic factor in idiopathic epilepsy. Symptomatic epilepsy usually develops as a result of a structural abnormality in the brain.

Status epilepticus is a particularly severe form of seizure, which is manifested as multiple seizures that persist for a significant length of time, or serial seizures without any recovery of consciousness between seizures. The overall mortality rate among adults with status epilepticus is approximately 20 percent. Patients who have a first episode are at substantial risk for future episodes and for the development of chronic epilepsy. The frequency of status epilepticus in the United States is approximately 150,000 cases per year, with approximately 55,000 deaths being associated with status epilepticus annually. Acute processes that are associated with status epilepticus include intractable epilepsy, metabolic disturbances (e.g. electrolyte abnormalities, renal failure and sepsis), central nervous system infection (meningitis or encephalitis), stroke, degenerative diseases, head trauma, drug toxicity and hypoxia. The fundamental pathophysiology of status epilepticus involves a failure of mechanisms that normally abort an isolated seizure. This failure can arise from abnormally persistent, excessive excitation or ineffective recruitment of inhibition. Studies have shown that excessive activation of excitatory amino acid receptors can cause prolonged seizures and suggest that excitatory amino acids may play a causative role. Status epilepticus can also be caused by penicillin and related compounds that antagonize the effects of γ-aminobutyric acid (GABA), the primary inhibitory neurotransmitter in the brain.

One early diagnostic procedure for epilepsy involved the oral administration of large quantities of water together with injections of vasopressin to prevent the accompanying diuresis. This procedure was found to induce seizures in epileptic patients, but rarely in non-epileptic individuals (Garland et al., Lancet, 2:566, 1943). Status epilepticus can be blocked in kainic acid-treated rats by intravenous injection of mannitol (Baran et al., Neuroscience, 21:679, 1987). This effect is similar to that achieved by intravenous injection of urea in human patients (Carter, Epilepsia, 3:198, 1962). The treatment in each of these cases increases the osmolarity of the blood and extracellular fluid, resulting in water efflux from the cells and an increase in extracellular space in the brain. Acetazolamide (ACZ), another diuretic with a different mechanism of action (inhibition of carbonic anhydrase), has been studied experimentally as an anticonvulsant (White et al., Advance Neurol., 44:695, 1986; and Guillaume et al., Epilepsia, 32:10, 1991) and used clinically on a limited basis (Tanimukai et al., Biochem. Pharm., 14:961, 1965; and Forsythe et al., Develop. Med. Child Neurol., 23:761, 1981). Although its mechanism of anticonvulsant action has not been determined, ACZ does have a clear effect on the cerebral extracellular space.

Traditional anti-epileptic drugs exert their principal effect through one of three mechanisms: (a) inhibition of repetitive, high-frequency neuronal firing by blocking voltage-dependent sodium channels; (b) potentiation of γ-aminobutyric acid (GABA)-mediated postsynaptic inhibition; and (c) blockade of T-type calcium channels.

Current anti-epileptic drug therapies exert their pharmacological effects on all brain cells, regardless of their involvement in seizure activity, Common side effects are over-sedation, dizziness, loss of memory and liver damage. Furthermore 20-30% of epilepsy patients are refractory to current therapy.

Migraine

Migraine headaches afflict 10-20% of the U.S. population, with an estimated loss of 64 million workdays annually. Migraine headache is characterized by pulsating head pain that is episodic, unilateral or bilateral, lasting from 4 to 72 hours and often associated with nausea, vomiting and hypersensitivity to light and/or sound. When accompanied by premonitory symptoms, such as visual, sensory, speech or motor symptoms, the headache is referred to as “migraine with aura,” formerly known as classic migraine. When not accompanied by such symptoms, the headache is referred to as “migraine without aura,” formerly known as common migraine. Both types evidence a strong genetic component, and both are three times more common in women than men. The precise etiology of migraine has yet to be determined. It has been theorized that persons prone to migraine have a reduced threshold for neuronal excitability, possibly due to reduced activity of the inhibitory neurotransmitter γ-aminobutyric acid (GABA). GABA normally inhibits the activity of the neurotransmitters serotonin (5-HT) and glutamate, both of which appear to be involved in migraine attacks. The excitatory neurotransmitter glutamate is implicated in an electrical phenomenon called cortical spreading depression, which can initiate a migraine attack, while serotonin is implicated in vascular changes that occur as the migraine progresses.

It has been suggested that cortical spreading depression (CSD) underlies migraine visual aura. CSD is characterized by a short burst of intense depolarization in the occipital cortex, followed by a wave of neuronal silence and diminished evoked potentials that advance anteriorly across the surface of the cerebral cortex. Enhanced excitability of the occipital-cortex neurons has been proposed as the basis for CSD. The visual cortex may have a lower threshold for excitability and therefore is most prone to CSD. It has been suggested that mitochondrial disorders, magnesium deficiency and abnormality of presynaptic calcium channels may be responsible for neuronal hyperexcitability (Welch, Pathogenesis of Migraine, Seminaras in Neurobiol., 17:4, 1997). During a spreading depression event, profound ionic perturbations occur, which include interstitial acidification, extracellular potassium accumulation and redistribution of sodium and chloride ions to intracellular compartments. In addition, prolonged glial swelling occurs as a homeostatic response to altered ionic extracellular fluid composition, and interstitial neurotransmitter and fatty acid accumulation. Studies have shown that furosemide inhibits regenerative cortical spreading depression in anaesthetized cats (Read et al, Cephalagia, 17:826, 1997).

Drug therapy is tailored to the severity and frequency of migraine headaches. For occasional attacks, abortive treatment may be indicated, but for attacks occurring two or more times per month, or when attacks greatly impact the patient's daily life, prophylactic therapy may be indicated.

Neurotoxicity

A variety of chemical and biological agents, as well as some infectious agents, have neurotoxic effects. A common example is the pathophysiological effect of acute ethanol ingestion. Episodic ethanol intoxications and withdrawals, characteristic of binge alcoholism, result in brain damage. Animal models designed to mimic the effects of alcohol in the human have demonstrated that a single dose of ethanol given for 5-10 successive days results in neurodegeneration in the entorhinal cortex, dentate gyrus and olfactory bulbs, accompanied by cerebrocortical edema and electrolyte (Na⁺ and K⁺) accumulation. As with other neurodegenerative conditions, research has focused primarily on synaptically based excitotoxic events involving excessive glutamatergic activity, increased intracellular calcium and decreased γ-aminobutyric acid.

Cognition, Learning and Memory

The cognitive abilities of mammals are thought to be dependent on cortical processing. It has generally been accepted that the most relevant parameters for describing and understanding cortical function are the spatio-temporal patterns of activity. In particular, long-term potentiation and long-term depression have been implicated in memory and learning and may play a role in cognition. Oscillatory and synchronized activities in the brains of mammals have been correlated with distinct behavioral states.

Synchronization of spontaneous neuronal firing activity is thought to be an important feature of a number of normal and pathophysiological processes in the central nervous system. Examples include synchronized oscillations of population activity such as gamma rhythms in the neocortex, which are thought to be involved in cognition (Singer and Gray, Annu. Rev. Neurosci., 18:855-86, 1995), and theta rhythm in hippocampus, which is thought to play roles in spatial memory and in the induction of synaptic plasticity (Heurta and Lisman, Neuron. 15:1053-63, 1995; Heurta and Lisman, J. Neurophysiol. 75:877-84, 1996; O'Keefe, Curr. Opin. Neurobiol., 3:917-24, 1993). To date, most research on the processes underlying the generation and maintenance of spontaneous synchronized activity has focused on synaptic mechanisms, However, there is evidence that nonsynaptic mechanisms may also play important roles in the modulation of synchronization in normal and pathological activities in the central nervous system.

Anxiety Disorders

Anxiety disorders are classified into several subtypes: Panic Disorder, Social Anxiety Disorder, Obsessive Compulsive Disorder, Posttraumatic Stress Disorder, Generalized Anxiety Disorder, and Specific Phobia. American Psychiatric Association. Diagnostic and Statistical Manual of Mental Disorders, 4^{th} edition (1994). All but the last of these are typically treated with various pharmacologic approaches as well as with psychotherapeutic approaches. As a group, the anxiety disorders have the highest prevalence in the U.S. of all psychiatric disorders. Kessler et al, Lifetime and 12-month prevalence of DSM-III-R psychiatric disorders in the United States: Results from the National Comorbidity Survey. Arch Gen Psychiatry 51:8-19 (1994). Anxiety disorders afflict 15.7 million people in the United States each year, and 30 million people in the United States at some point in their lives. Lepine J P. The Epidemiology of Anxiety Disorders: Prevalence and Societal Costs. J. Clin. Psychiatry. 63: Suppl 14:4-8 (2002).

The most commonly prescribed pharmacologic treatments for anxiety are the selective serotonin reuptake inhibitors (SSRIs); however, other antidepressant drugs are also used, and benzodiazepines are frequently prescribed to treat acute anxiety and to treat Panic Disorder. Antiepileptic drugs have also been used in the treatment of Posttraumatic Stress Disorder. Many of the treatments, such as SSRIs, are used for most of the anxiety disorder subtypes. Although treatment with different compounds such as tricyclic antidepressants, selective serotonin reuptake inhibitors, high-potency benzodiazepines, and monoamine oxidase inhibitors has been proven effective in anxiety disorders, 20% to 40% of patients are nonresponders. Denys D. and de Geus F. Predictors of Pharmacotherapy Response in Anxiety Disorders. Curr Psychiatry Rep. 7(4):252-7 (August 2005). Additionally, there are numerous side effects associated with long-term use of SSRIs, such as sexual dysfunction and weight gain. Hirschfeld R M. long-term Side Effects of SSRIs: Sexual Dysfunction and Weight Gain. J Clin. Psychiatry. 64: Suppl 18:20-4 (2003). Hence there is a great need for improved anxiety therapeutics.

Addictive Disorders

Addictive and/or compulsive disorders, such as eating disorders (including obesity), addiction to narcotics/physical dependence, alcoholism, and smoking are a major public health problem that impacts society on multiple levels. It has been estimated that substance abuse costs the US more than $484 billion per year. Current strategies for the treatment of additive disorders include psychological counseling and support, use of therapeutic agents or a combination of both. A variety of agents known to affect the central nervous system have been used in various contexts to treat a number of indications related directly or indirectly to addictive behaviors.

Neuropathic Pain

Neuropathic pain and nociceptive pain differ in their etiology, pathophysiology, diagnosis and treatment. Nociceptive pain occurs in response to the activation of a specific subset of peripheral sensory neurons, the nociceptors. It is generally acute (with the exception of arthritic pain), self-limiting and serves a protective biological function by acting as a warning of on-going tissue damage. It is typically well localized and often has an aching or throbbing quality. Examples of nociceptive pain include post-operative pain, sprains, bone fractures, burns, bumps, bruises, inflammation (from an infection or arthritic disorder), obstructions and myofascial pain. Nociceptive pain can usually be treated with opioids and non-steroidal anti-inflammatory drugs (NSAIDS).

Neuropathic pain is a common type of chronic, non-malignant, pain, which is the result of an injury or malfunction in the peripheral or central nervous system and serves no protective biological function. It is estimated to affect more than 1.6 million people in the U.S. population. Neuropathic pain has many different etiologies, and may occur, for example, due to trauma, diabetes, infection with herpes zoster (shingles), HIV/AIDS, late-stage cancer, amputation (including mastectomy), carpal tunnel syndrome, chronic alcohol use, exposure to radiation, and as an unintended side-effect of neurotoxic treatment agents, such as certain anti-HIV and chemotherapeutic drugs.

In contrast to nociceptive pain, neuropathic pain is frequently described as “burning”, “electric”, “tingling” or “shooting” in nature. It is often characterized by chronic allodynia (defined as pain resulting from a stimulus that does not ordinarily elicit a painful response, such as light touch) and hyperalgesia (defined as an increased sensitivity to a normally painful stimulus), and may persist for months or years beyond the apparent healing of any damaged tissues.

Neuropathic pain can be difficult to treat. Analgesic drugs that are effective against normal pain (e.g., opioid narcotics and non-steroidal anti-inflammatory drugs) are rarely effective against neuropathic pain. Similarly, drugs that have activity in neuropathic pain are not usually effective against nociceptive pain. The standard drugs that have been used to treat neuropathic pain appear to often act selectively to relieve certain symptoms but not others in a given patient (for example, relief of allodynia, but not hyperalgesia). For this reason, it has been suggested that successful therapy may require the use of multiple different combinations of drugs and individualized therapy (see, for example, Bennett, Hosp. Pract. (Off Ed). 33:95-98, 1998). Treatment agents typically employed in the management of neuropathic pain include tricylic antidepressants (for example, amitriptyline, imipramine, desimipramine and clomipramine), systemic local anesthetics, and anti-convulsants (such as phenytoin, carbamazepine, valproic acid, clonazepam and gabapentin).

Many anti-convulsants originally developed for the treatment of epilepsy and other seizure disorders have found application in the treatment of non-epileptic conditions, including neuropathic pain, mood disorders (such as bipolar affective disorder), and schizophrenia (for a review of the use of anti-epileptic drugs in the treatment of non-epileptic conditions, see Rogawski and Loscher, Nat. Medicine, 10:685-692, 2004). It has thus been suggested that epilepsy, neuropathic pain and affective disorders have a common pathophysiological mechanism (Rogawski & Loscher, ibid; Ruscheweyh & Sandkuhler, Pain 105:327-338, 2003), namely a pathological increase in neuronal excitability, with a corresponding inappropriately high frequency of spontaneous firing of neurons. However, only some, and not all, antiepileptic drugs are effective in treating neuropathic pain, and furthermore such antiepileptic drugs are only effective in certain subsets of patients with neuropathic pain (McCleane, Expert. Opin. Pharmacother. 5:1299-1312, 2004).

The focus of pharmacological intervention in many disorders of the central and peripheral nervous system, including neuropathic pain, has been on reducing neuronal hyperexcitability. Most agents currently used to treat such disorders target synaptic activity in excitatory pathways by, for example, modulating the release or activity of excitatory neurotransmitters, potentiating inhibitory pathways, blocking ion channels involved in impulse generation, and/or acting as membrane stabilizers. Conventional agents and therapeutic approaches for the treatment of central and peripheral nervous system disorders thus reduce neuronal excitability and inhibit synaptic firing. One serious drawback of these therapies is that they are nonselective and exert their actions on both normal and abnormal neuronal populations. This leads to negative and unintended side effects, which may affect normal CNS functions, such as cognition, learning and memory, and produce adverse physiological and psychological effects in the treated patient. Common side effects include over-sedation, dizziness, loss of memory and liver damage.

Accordingly, there is a continuing need for compositions and methods for regulating various CNS disorders and maintaining and/or enhancing normal CNS function that involve therapies capable of traversing the blood-brain barrier.

### SUMMARY OF THE INVENTION

The present invention provides compounds that traverse the blood-brain barrier. Embodiments of the present invention provide compounds according to formula I, II, III, IV, V and/or VI:

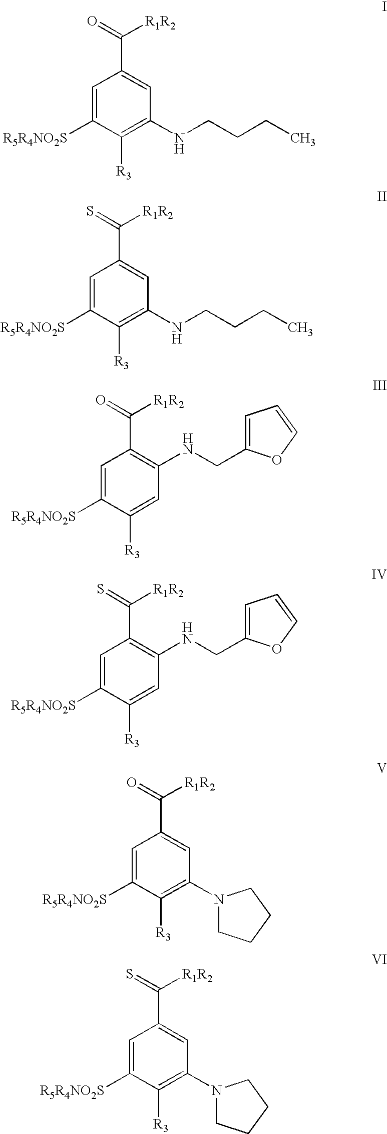

or a pharmaceutically acceptable salt, solvate, tautomer or hydrate thereof, wherein

R₁ is not present, H, O or S;

R₂ is not present, H or when R₁ is O or S, R₂ is selected from the group consisting of hydrogen, alkyl, aralkyl, aryl, alkylaminodialkyl, alkylcarbonylaminodialkyl, alkyloxycarbonylalkyl, alkylcarbonyloxyalkyl, alkylaldehyde, alkylketoalkyl, alkylamide, alkarylamide, arylamide, an alkylammonium group, alkylcarboxylic acid, alkylheteroaryl, alkylhydroxy, a biocompatible polymer such as alkyl(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, and when R₁ is not present, R₂ is selected from the group consisting of hydrogen, N,N-dialkylamino, N,N-dialkarylamino, N,N-diarylamino, N-alkyl-N-alkarylamino, N-alkyl-N-arylamino, N-alkaryl-N-arylamino, unsubstituted or substituted;

R₃ is selected from the group consisting of aryl, halo, hydroxy, alkoxy, and aryloxy, unsubstituted or substituted; and

R₄ and R₅ are each independently selected from the group consisting of hydrogen, alkylaminodialkyl, carbonylalkyl, carbonylalkaryl, carbonylaryl, and salts thereof such as sodium, potassium, calcium, ammonium, trialkylarylammonium and tetraalkylammonium salts, with the following provisos in some embodiments: R₃ of formula I is not phenyloxy when R₁ is O and R₂, R₄ and R₅ are H, more specifically, in some embodiments, the compound of formula I is not bumetanide; R₃ of formula III is not Cl, when R₁ is O and R₂, R₄ and R₅ are H, more specifically, in some embodiments, the compound of formula III is not furosemide; R₂ of formula III is not methyl when R₁ is O, R₃ is Cl, and R₄ and R₅ are H, more specifically, in some embodiments, the compound of formula III is not furosemide methyl ester; R₃ of formula V is not phenyloxy when R₁ is O and R₂, R₄ and R₅ are H, more specifically, in some embodiments, the compound of formula V is not piretanide.

Embodiments of the present invention provide compounds according to formula VII:

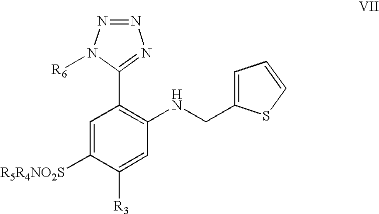

or a pharmaceutically acceptable salt, solvate, tautomer or hydrate thereof, wherein

R₃, R₄ and R₅ are defined above; and

R₆ is selected from the group consisting of alkyloxycarbonylalkyl, alkylaminocarbonylalkyl, alkylaminodialkyl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, with the proviso that, in some embodiments, R₃ of formula VII is not Cl, when R₄, R₅ and R₆ are H, more specifically, in some embodiments, the compound of formula VII is not azosemide.

Embodiments of the present invention further provide compounds according to formula VIII:

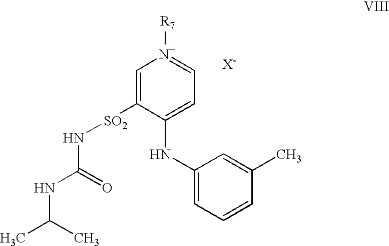

or a pharmaceutically acceptable salt, solvate, tautomer or hydrate thereof,

wherein

R₇ is not present or selected from the group consisting of hydrogen, alkyloxycarbonylalkyl, alkylaminocarbonylalkyl, alkylaminodialkyl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted; and

X⁻ is a halide such as bromide, chloride, fluoride, iodide or an anionic moiety such as mesylate or tosylate; alternatively, X⁻ is not present and the compound forms an “inner” or zwitterionic salt (where R₇ is H), with the proviso that, in some embodiments, R₇ is always present and X⁻ is not present. More specifically, in some embodiments, the compound of formula VIII is not torsemide.

Embodiments of the present invention provide prodrugs capable of passage across the blood-brain barrier comprising a compound of formula I, II, III, IV, V, VI, VII and/or VIII, or a pharmaceutically acceptable salt, solvate, tautomer or hydrate thereof. In some embodiments, the compound of the prodrug is provided in an amount effective for regulating a CNS disorder. In particular embodiments, the CNS disorder is epilepsy, anxiety, neuropathic pain, neural function, drug addiction/physical dependence and/or migraines.

Embodiments of the present invention provide a pharmaceutical composition comprising a compound of formula I, II, III, IV, V, VI, VII and/or VIII, or a pharmaceutically acceptable salt, solvate, tautomer, hydrate or combination thereof and a pharmaceutically acceptable carrier, excipient or diluent. In some embodiments, the compound of the pharmaceutical composition is present in an amount effective for regulating a CNS disorder. In particular embodiments, the CNS disorder is epilepsy, anxiety, neuropathic pain, neural function and/or migraines.

Embodiments of the present invention provide methods of making the compounds described herein and further provide intermediate compounds formed through the synthetic methods described herein to provide the compounds of formula I, II, III, IV, V, VI, VII and/or VIII.

Embodiments of the present invention provide kits including the compounds described herein.

Embodiments of the present invention provide uses of the compounds described herein for the preparation of a medicament for carrying out the aforementioned utilities. In particular embodiments. the CNS disorder is epilepsy, anxiety, neuropathic pain, neural function and/or migraines.

Embodiments of the present invention further provide methods of regulating a CNS disorder. In particular, compounds of formula I, II, III, IV, V, VI, VII and/or VIII of the present invention as well as the prodrugs and modified diuretic or diuretic-like compounds described herein can be used for the regulation, including prevention, management and treatment, of a range of CNS conditions.

The foregoing and other objects and aspects of the present invention are explained in greater detail in reference to the drawing and description set forth herein.

### BRIEF DESCRIPTION OF THE DRAWINGS

FIG. 1 presents a graph depicting the results of bumetanide analogs on the difference in startle amplitude in comparison to control as a measure of the ability of the bumetanide analogs to alleviate anxiety.

### DETAILED DESCRIPTION

The foregoing and other aspects of the present invention will now be described in more detail with respect to embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. Furthermore, the term “about,” as used herein when referring to a measurable value such as an amount of a compound, dose, time, temperature, and the like, is meant to encompass variations of 20%, 10%, 5%, 1%, 0.5%, or even 0.1% of the specified amount. Unless otherwise defined, all terms, including technical and scientific terms used in the description, have the same meaning as commonly understood by one of ordinary skill in the art to which this invention belongs.

All publications, patent applications, patents, and other references mentioned herein are incorporated by reference in their entirety.

The term “alkyl” as used herein refers to a straight or branched chain saturated or partially unsaturated hydrocarbon radical. Examples of alkyl groups include, but are not limited to, methyl, ethyl, isopropyl, tert-butyl, n-pentyl and the like. By “unsaturated” is meant the presence of 1, 2 or 3 double or triple bonds, or a combination thereof. Such alkyl groups may be optionally substituted as described herein.

The term “alkaryl” as used herein refers to a straight or branched chain, saturated or partially unsaturated hydrocarbon radical bonded to an aryl group. Examples of alkaryl groups include, but are not limited to, benzyl, 4-chlorobenzyl, methylbenzyl, dimethylbenzyl, ethylphenyl, propyl-(4-nitrophenyl), and the like. Such alkaryl groups may be optionally substituted as described herein.

The term “alkylene” as used herein refers to a straight or branched chain having two terminal monovalent radical centers derived by the removal of one hydrogen atom from each of the two terminal carbon atoms of straight-chain parent alkane.

The term “aryl” or Ar as used herein refers to an aromatic group, a heteroaryl group or to an optionally substituted aromatic group or heteroaryl group fused to one or more optionally substituted aromatic groups or heteroaryl groups, optionally substituted with suitable substituents including, but not limited to, lower alkyl, lower alkoxy, lower alkylsulfanyl, lower alkylsulfenyl, lower alkylsulfonyl, oxo, hydroxy, mercapto, amino optionally substituted by alkyl, carboxy, tetrazolyl, carbamoyl optionally substituted by alkyl, aminosulfonyl optionally substituted by alkyl, acyl, aroyl, heteroaroyl, acyloxy, aroyloxy, heteroaroyloxy, alkoxycarbonyl, nitro, cyano, halogen, or lower perfluoroalkyl, multiple degrees of substitution being allowed. Examples of aryl include, but are not limited to, phenyl, 2-naphthyl, 1-naphthyl, 4-pyridyl and the like.

The term “halo” as used herein refers to bromo, chloro, fluoro or iodo. Alternatively, the term “halide” as used herein refers to bromide, chloride, fluoride or iodide.

The term “hydroxy” as used herein refers to the group —OH.

The term “alkoxy” as used herein alone or as part of another group, refers to an alkyl group, as defined herein, appended to the parent molecular moiety through an oxy group. Representative examples of alkoxy include, but are not limited to, methoxy, ethoxy, propoxy, 2-propoxy, butoxy, tert-butoxy, pentyloxy, hexyloxy and the like.

The terms “alkaryloxy” or “oxyalkaryl” as used herein refers to the group —O— alkyl-aryl wherein Ar is aryl. Examples include, but are not limited to, benzyloxy, oxybenzyl, 2-naphthyloxy and oxy-2-naphthyl.

The term “aryloxy” as used herein refers to the group —ArO wherein Ar is aryl or heteroaryl. Examples include, but are not limited to, phenoxy, benzyloxy and 2-naphthyloxy.

The term “amino” as used herein refers to —NH₂ in which one or both of the hydrogen atoms may optionally be replaced by alkyl or aryl or one of each, optionally substituted.

The terms “alkylthio” or “thioalkyl” as used herein alone or as part of another group, refers to an alkyl group, as defined herein, appended to the parent molecular moiety through a sulfur moiety. Representative examples of alkylthio include, but are not limited to, methylthio, thiomethyl, ethylthio, thioethyl, n-propylthio, thio-n-propyl, isopropylthio, thio-isopropyl, n-butylthio, thio-n-butyl and the like.

The terms “arylthio” or “thioaryl” as used herein refers to the group —ArS wherein Ar is aryl. Examples include, but are not limited to, phenylthio, thiophenyl, 2-naphthylthio and thio-2-naphthyl.

The terms “alkarylthio” or “thioalkaryl” as used herein refers to the group —S— alkyl-aryl wherein Ar is aryl. Examples include, but are not limited to, benzyllthio, tlhiobenzyl, 2-naphthylthio and thio-2-naphthyl.

The term “carboxy” as used herein refers to the group —COO₂H.

The term “quaternary ammonium” as used herein refers to a chemical structure having four bonds to the nitrogen with a positive charge on the nitrogen in the “ionium” state, i.e., “R₄N⁺” or “quaternary nitrogen,” wherein R is an organic substituent such as alkyl or aryl. The term “quaternary ammonium salt” as used herein refers to the association of the quaternary ammonium cation with an anion.

The term “substituted” as used herein refers to replacement of one or more of the hydrogen atoms of the group replaced by substituents known to those skilled in the art and resulting in a stable compound as described below. Examples of suitable replacement groups include, but are not limited to, alkyl, acyl, alkenyl, alkynyl cycloalkyl, aryl, alkaryl, hydroxy, thio, alkoxy, aryloxy, acyl, amino, amido, carboxy, carboxyalkyl, thiocarboxyalkyl, carboxyaryl, thiocarboxyaryl, halo, oxo, mercapto, sulfinyl, sulfonyl, sulfonamido, amidino, carbamoyl, cycloalkyl, heterocycloalkyl, dialkylaminoalkyl, carboxylic acid, carboxamido, haloalkyl, dihaloalkyl, trihaloalkyl, trihaloalkoxy, alkylthio, aralkyl, alkylsulfonyl, arylthio, amino, alkylamino, dialkylamino, guanidino, ureido, nitro and the like. Substitutions are permissible when such combinations result in compounds stable for the intended purpose. For example, substitutions are permissible when the resultant compound is sufficiently robust to survive isolation to a useful degree of purity from a reaction mixture, and formulation into a therapeutic or diagnostic agent or reagent.

The term “effective amount” or “effective” is intended to designate a dose that causes a relief of symptoms of a disease or disorder as noted through clinical testing and evaluation, patient observation, and/or the like. “Effective amount” or “effective” further can further designate a dose that causes a detectable change in biological or chemical activity. The detectable changes may be detected and/or further quantified by one skilled in the art for the relevant mechanism or process. Moreover, “effective amount” or “effective” can designate an amount that maintains a desired physiological state, i.e., reduces or prevents significant decline and/or promotes improvement in the condition of interest. As is generally understood in the art, the dosage will vary depending on the administration routes, symptoms and body weight of the patient but also depending upon the compound being administered.

The term “solvate” as used herein is intended to refer to a pharmaceutically acceptable solvate form of a specified compound that retains the biological effectiveness of such compound, for example, resulting from a physical association of the compound with one or more solvent molecules. Examples of solvates, without limitation, include compounds of the invention in combination with water, 1-propanol, 2-propanol, ethanol, methanol, DMSO, ethyl acetate, acetic acid, or ethanolamine.

The term “hydrate” as used herein refers to the compound when the solvent is water.

The term “biocompatible polymer” as used herein refers to a polymer moiety that is substantially non-toxic and does not tend to produce substantial immune responses, clotting or other undesirable effects. Accordingly to some embodiments of the present invention, polyalkylene glycol is a biocompatible polymer where, as used herein, polyalkylene glycol refers to straight or branched polyalkylene glycol polymers such as polyethylene glycol, polypropylene glycol, and polybutylene glycol, and further includes the monoalkylether of the polyalkylene glycol. In some embodiments of the present invention, the polyalkylene glycol polymer is a lower alkyl polyalkylene glycol moiety such as a polyethylene glycol moiety (PEG), a polypropylene glycol moiety, or a polybutylene glycol moiety. PEG has the formula —HO(CH₂CH₂O)ₙH, where n can range from about 1 to about 4000 or more. In some embodiments, n is 1 to 100, and in other embodiments, n is 5 to 30. The PEG moiety can be linear or branched. In further embodiments, PEG can be attached to groups such as hydroxyl, alkyl, aryl, acyl or ester. In some embodiments, PEG can be an alkoxy PEG, such as methoxy-PEG (or mPEG), where one terminus is a relatively inert alkoxy group, while the other terminus is a hydroxyl group.

1. Compounds

According to some embodiments, the present invention provides novel compounds. Thus, any of the R groups as defined herein can be excluded or modified in order to exclude a known compound and/or provide a novel compound. Compounds of the present invention can include compounds according to formula I, II, III, IV, V, VI, VII and/or VIII:

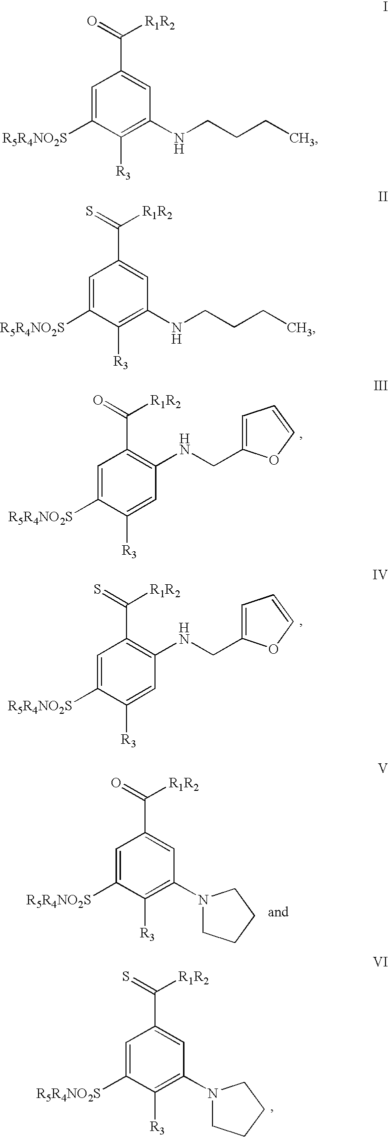

or a pharmaceutically acceptable salt, solvate, tautomer or hydrate thereof, wherein

R₁ is not present, H, O or S;

R₂ is not present, H or when R₁ is O or S, R₂ is selected from the group consisting of hydrogen, alkyl, aralkyl, aryl, alkylaminodialkyl, alkylcarbonylaminodialkyl, alkyloxycarbonylalkyl, alkylcarbonyloxyalkyl, alkylaldehyde, alkylketoalkyl, alkylamide, alkarylamide, arylamide, an alkylammonium group, alkylcarboxylic acid, alkylheteroaryl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, and when R₁ is not present, R₂ is selected from the group consisting of hydrogen, N,N-dialkylamino, N,N-dialkarylamino, N,N-diarylamino, N-alkyl-N-alkarylamino, N-alkyl-N-arylamino, N-alkaryl-N-arylamino, unsubstituted or substituted;

R₃ is selected from the group consisting of aryl, halo, hydroxy, alkoxy, and aryloxy, unsubstituted or substituted; and

R₄ and R₅ are each independently selected from the group consisting of hydrogen, alkylaminodialkyl, carbonylalkyl, carbonylalkaryl, carbonylaryl, and salts thereof such as sodium, potassium, calcium, ammonium, trialkylarylammonium and tetraalkylammonium salts, with the following provisos in some embodiments: R₃ of formula I is not phenyloxy when R₁ is O and R₂, R₄ and R₅ are H, more specifically, in some embodiments, the compound of formula I is not bumetanide; R₃ of formula III is not Cl, when R₁ is O and R₂, R₄ and R₅ are H, more specifically, in some embodiments, the compound of formula III is not furosemide; R₂ of formula III is not methyl when R₁ is O, R₃ is Cl, and R₄ and R₅ are H, more specifically, in some embodiments, the compound of formula III is not furosemide methyl ester; R₃ of formula V is not phenyloxy when R₁ is O and R₂, R₄ and R₅ are H, more specifically, in some embodiments, the compound of formula V is not piretanide

In some embodiments of the present invention, the compound of formula I can be bumetanide, bumetanide aldehyde, bumetanide methyl ester, bumetanide cyanomethyl ester, bumetanide ethyl ester, bumetanide isoamyl ester, bumetanide octyl ester, bumetanide benzyl ester, bumetanide dibenzylamide, bumetanide diethylamide, bumetanide morpholinoethyl ester, bumetanide 3-(dimethylaminopropyl)ester, bumetanide N,N-diethylglycolamido ester, bumetanide N,N-dimethylglycolamido ester, bumetanide pivaxetil ester, bumetanide propaxetil ester, bumetanide methoxy(polyethyleneoxy)ₙ₋₁-ethyl ester, bumetanide benzyltrimethylammonium salt and bumetanide cetyltrimethylammonium salt. In particular embodiments, the compound is not bumentanide.

In other embodiments of the present invention, the compound of formula I can be bumetanide[—(C—O)—SH]thioacid, bumetanide S-methyl thioester, bumetanide S-cyanomethyl thioester, bumetanide S-ethyl thioester, bumetanide S-isoamyl thioester, bumetanide S-octyl thioester, bumetanide S-benzyl thioester, bumetanide S-(morpholinoethyl)thioester, bumetanide S-[3-(dimethylaminopropyl)]thioester, bumetanide S-(N,N-diethylglycolamido)thioester, bumetanide S-(N,N-dimethylglycolamido)thioester, bumetanide S-pivaxetil thioester, bumetanide S-propaxetil thioester, bumetanide S-[methoxy(polyethyleneoxy)ₙ₋₁-ethyl]thioester, bumetanide[—(C═O)—S⁻]benzyltrimethylammonium thioacid salt and bumetanide[—(C═O)—S⁻]cetyltrimethylammonium thioacid salt.

In some embodiments of the present invention, the compound of formula II can be metastable bumetanide[—(C═S)—OH]thioacid, bumetanide O-methyl thioester, bumetanide O-cyanomethyl thioester, bumetanide O-ethyl thioester, bumetanide O-isoamyl thioester, bumetanide O-octyl thioester, bumetanide O-benzyl thioester, bumetanide O-(morpholinoethyl) thioester, bumetanide O-[3-(dimethylaminopropyl)]thioester, bumetanide O-(N,N-diethylglycolamido) thioester, bumetanide, O-(N,N-dimethylglycolamido) thioester, bumetanide O-pivaxetil thioester, bumetanide O-propaxetil thioester, bumetanide O-[methoxy(polyethyleneoxy)ₙ₋₁-ethyl]thioester, bumetanide[—(C═S)—O⁻]benzyltrimethylammonium thioacid salt and bumetanide[—(C═S)—O⁻]cetyltrimethylammonium thioacid salt.

In some embodiments of the present invention, the compound of formula II can be bumetanide thiioaldehyde, bumetanide[—(C═S)—SH]dithioacid, bumetanide methyl dithioester, bumetanide cyanomethyl dithioester, bumetanide ethyl dithioester, bumetanide isoamyl dithioester, bumetanide octyl dithioester, bumetanide benzyl dithioester, bumetanide dibenzylthioamide, bumetanide diethylthioamide, bumetanide morpholinoethyl dithioester, bumetanide 3-(dimethylaminopropyl) dithioester, bumetanide N,N-diethylglycolamido dithioester, bumetanide N,N-dimethylglycolamido dithioester, bumetanide pivaxetil dithioester, bumetanide propaxetil dithioester, bumetanide methoxy(polyethyleneoxy)ₙ₋₁-ethyl dithioester, bumetanide benzyltrimethylammonium dithioacid salt and bumetanide cetyltrimethylammonium dithioacid salt.

In other embodiments of the present invention, the compound of formula III can be furosemide, furosemide aldehyde, furosemide methyl ester, furosemide cyanomethyl ester, furosemide ethyl ester, furosemide isoamyl ester, furosemide octyl ester, furosemide benzyl ester, furosemide morpholinoethyl ester, furosemide 3-(dimethylaminopropyl) ester, furosemide N,N-diethylglycolamido ester, furosemide N,N-dimethylglycolamido ester, furosemide pivaxetil ester, furosemide propaxetil ester, furosemide methoxy(polyethyleneoxy)ₙ₋₁-ethyl ester, furosemide benzyltrimethylammonium acid salt and furosemide cetyltrimethylammonium acid salt. In particular embodiments, the compound is not furosemide.

In further embodiments of the present invention, the compound of formula III can be furosemide[—(C═O)—SH]thioacid, furosemide S-methyl thioester, furosemide S-cyanomethyl thioester, furosemide S-ethyl thioester, furosemide S-isoamyl thioester, furosemide S-octyl thioester, furosemide S-benzyl thioester, furosemide S-(morpholinoethyl) thioester, furosemide S-[3-(dimethylaminopropyl)]thioester, furosemide S-(N,N-diethylglycolamido) thioester, furosemide S-(N,N-dimethylglycolamido) thioester, furosemide S-pivaxetil thioester, furosemide S-propaxetil thioester, furosemide S-[methoxy(polyethyleneoxy)ₙ₋₁-ethyl]thioester, furosemide[—(C═O)—S⁻]benzyltrimethylammonium thioacid salt and furosemide[—(C═O)—S⁻]cetyltrimethylammonium thioacid salt.

In other embodiments of the present invention, the compound of formula IV can be metastable furosemide[—(C═S)—OH]thioacid, furosemide O-methyl thioester, furosemide O-cyanomethyl thioester, furosemide O-ethyl thioester, furosemide O-isoamyl thioester, furosemide O-octyl thioester, furosemide O-benzyl thioester, furosemide O-(morpholinoethyl) thioester, furosemide O-[3-(dimethylaminopropyl)]thioester, furosemide O-(N,N-diethylglycolamido) thioester, furosemide O-(N,N-dimethylglycolamido) thioester, furosemide O-pivaxetil thioester, furosemide O-propaxetil thioester, furosemide O-[methoxy(polyethyleneoxy)ₙ₋₁-ethyl]thioester, furosemide[—(C═S)—O⁻]benzyltrimethylammonium thioacid salt and furosemide[—(C═S)—O⁻]cetyltrimethylammonium thioacid salt.

In further embodiments of the present invention, the compound of formula IV can be furosemide thioaldehyde, furosemide[—(C═S)—SH]dithioacid, furosemide methyl dithioester, furosemide cyanomethyl dithioester, furosemide ethyl dithioester, furosemide isoamyl dithioester, furosemide octyl dithioester, furosemide benzyl dithioester, furosemide dibenzylthioamide, furosemide diethylthioamide, furosemide morpholinoethyl dithioester, furosemide 3-(dimethylaminopropyl) dithioester, furosemide N,N-diethylglycolamido dithioester, furosemide N,N-dimethylglycolamido dithioester, furosemide pivaxetil dithioester, furosemide propaxetil dithioester, furosemide methoxy(polyethyleneoxy)ₙ₋₁-ethyl dithioester, furosemide benzyltrimethylammonium dithioacid salt and furosemide cetyltrimethylammonium dithioacid salt.

In still further embodiments of the present invention, the compound of formula V can be piretanide, piretanide aldehyde, piretanide methyl ester, piretanide cyanomethyl ester, piretanide ethyl ester, piretanide isoamyl ester, piretanide octyl ester, piretanide benzyl ester, piretanide dibenzylamide, piretanide diethylamide, piretanide morpholinoethyl ester, piretanide 3-(dimethylaminopropyl) ester, piretanide N,N-diethylglycolamide ester, piretanide dimetlylglycolamide ester, piretanide pivaxetil ester, piretanide propaxetil ester, piretanide methoxy(polyethyleneoxy)ₙ₋₁-ethyl ester, piretanide benzyltrimethylammonium salt and piretanide cetyltrimethylammonium salt. In particular embodiments, the compound is not piretinide.

In some embodiments of the present invention, the compound of formula V can be piretanide[—(C═O)—SH]thioacid, piretanide S-methyl thioester, piretanide S-cyanomethyl thioester, piretanide S-ethyl thioester, piretanide S-isoamyl thioester, piretanide S-octyl thioester, piretanide S-benzyl thioester, piretanide S-(morpholinoethyl) thioester, piretanide S-[3-(dimethylaminopropyl)]thioester, piretanide S-(N,N-diethylglycolamido) thioester, piretanide S-(N,N-dimethylglycolamido) thioester, piretanide S-pivaxetil thioester, piretanide S-propaxetil thioester, piretanide S-[methoxy(polyethyleneoxy)ₙ₋₁-ethyl]thioester, piretanide[—(C═O)—S⁻]benzyltrimethylammonium thioacid salt and piretanide[—(C═O)—S⁻]cetyltrimethylammonium thioacid salt.

In further embodiments of the present invention, the compound of formula VI can be metastable piretanide[—(C═S)—OH]thioacid, piretanide O-methyl thioester, piretanide O-cyanomethyl thioester, piretanide O-ethyl thioester, piretanide O-isoamyl thioester, piretanide O-octyl thioester, piretanide O-benzyl thioester, piretanide O-(morpholinoethyl) thioester, piretanide O-[3-(dimethylaminopropyl)]thioester, piretanide O-(N,N-diethylglycolamido) thioester, piretanide, O-(N,N-dimethylglycolamido) thioester, piretanide O-pivaxetil thioester, piretanide O-propaxetil thioester, piretanide O-[methoxy(polyethyleneoxy)ₙ₋₁-ethyl]thioester, piretanide[—(C═S)—O⁻]benzyltrimethylammonium thioacid salt and piretanide[—(C═S)—O⁻]cetyltrimethylammonium thioacid salt.

In some embodiments of the present invention, the compound of formula VI can be piretanide thioaldehyde, piretanide[—(C═S)—SH]dithioacid, piretanide methyl dithioester, piretanide cyanomethyl dithioester, piretanide ethyl dithioester, piretanide isoamyl dithioester, piretanide octyl dithioester, piretanide benzyl dithioester, piretanide dibenzylthioamide, piretanide diethylthioamide, piretanide morpholinoethyl dithioester, piretanide 3-(dimethylaminopropyl) dithioester, piretanide N,N-diethylglycolamido dithioester, piretanide N,N-dimethylglycolamido dithioester, piretanide pivaxetil dithioester, piretanide propaxetil dithioester, piretanide methoxy(polyethyleneoxy)ₙ₋₁-ethyl dithioester, piretanide benzyltrimethylammonium dithioacid salt and piretanide cetyltrimethylammonium dithioacid salt.

In other embodiments of the present invention, the compound of formula VII can be tetrazolyl-substituted azosemides (such as methoxymethyl tetrazolyl-substituted azosemides, methylthiomethyl tetrazolyl-substituted azosemides and N-mPEG350-tetrazolyl-substituted azosemides), azosemide benzyltrimethylammonium salt and/or azosemide cetyltrimethylammonium salt.

In some embodiments of the present invention, the compound of formula VIII can be pyridine-substituted torsemide quaternary ammonium salts or the corresponding inner salts (zwitterions). Examples include, but are not limited to, methoxymethyl pyridinium torsemide salts, methylthiomethyl pyridinium torsemide salts and N-mPEG350-pyridinium torsemide salts.

Embodiments of the present invention further provide intermediate compounds formed through the synthetic methods described herein to provide the compounds of formula I, II, III, IV, V, VI, VII and/or VIII. The intermediate compounds may possess utility as therapeutic agents for the range of indications described herein and/or reagents for further synthesis methods and reactions.

As noted previously, any of the R groups as defined herein can be excluded from the compounds of the present invention, particularly with reference to denoting novel compounds of the present invention.

2. Synthetic Methods

Embodiments of the present invention provide methods of modifying diuretic or diuretic-like compounds to increase lipophilicity of the diuretic or diuretic-like compounds. In some embodiments, the compound is a diuretic or diuretic-like compound, and in particular embodiments, the compound is termed a “loop diuretic.” For a discussion of pharmacological properties of diuretics, see generally, Goodman & Gilman's The Pharmacological Basis of Therapeutics, Hardman, J. G., Limbird, L. E. and Gilman, A. G., Eds., McGraw-Hill Medical Publishing Division (10th ed. 2001).

Further included as a diuretic or diuretic-like compound are cation-chloride cotransporters. As used herein, a cotransporter is electroneutral, moving equal amounts of oppositely charged ionic species from one side of a membrane to another. As used herein, a cation-chloride cotransporter refers to a cotransporter that moves one or several cations with an equal number of chloride ions. Exemplary cation chloride cotransporters include, but are not limited to, the loop diuretic-sensitive Na⁺, K⁺, 2Cl⁻ cotransporter in the brain (NKCCl), and the thiazide-sensitive Na⁺, Cl⁻ cotransporter (NCC). Discussions regarding the molecular classification of cation-chloride cotransporters, their physiology, and pharmacology can be found in Mount, D. B., Delpire E., Gamba G., Hall A. E., Poch E., Hoover R. S., Hebert S. C.: The electroneutral cation-chloride cotransporters. J Exp Biol 201: 2091-2102, 1998 and Russell J. M. Sodium-potassium-chloride cotransport. Physiol Rev. 2000 January; 80(1):211-76.

The NKCC1 brain-specific cotransporter is an isoform of its kidney analog, NKCC2. Furosemide and bumetanide are classic examples of NKCC antagonists.

The thiazide-sensitive cotransporter is antagonized by thiazide diuretics. Exemplary thiazide diuretics include, but are not limited to, chlorothiazide, hydrochlorothiazide, and benzthiazide.

Modification of the diuretic or diuretic-like compound can include reacting the diuretic or diuretic-like compound with a functional group and/or compound selected from the group consisting of an aluminum hydride, alkyl halide, alcohol, aldehyde, alkaryl halide, mono- and dialkylamine, mono- andi dialkarylamine, mono- and diarylamine, and quaternary ammonium salt, unsubstituted or substituted, or combinations thereof. Non-limiting examples of compounds that may be used as a starting material are exemplified below.

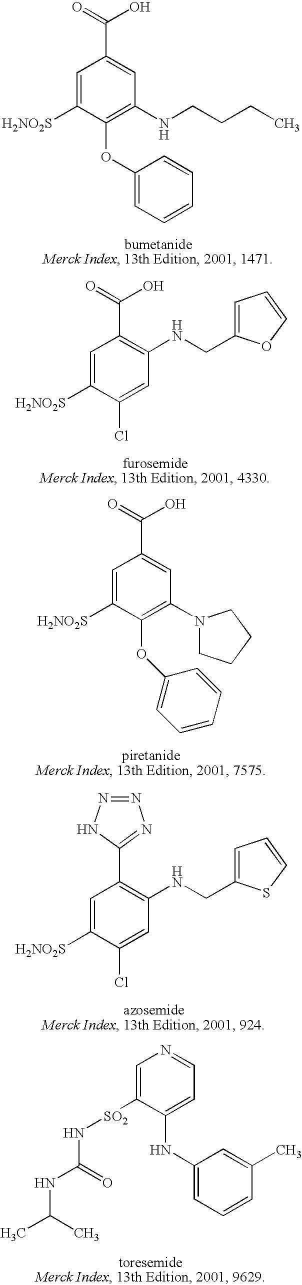

The compounds of formula I, II, III, IV, V, VI, VII and/or VIII can be synthesized using traditional synthesis techniques well known to those skilled in the art. More specific synthesis routes are described below.

A. Bumetanide Analogs, Thiobumetanide Analogs and Dithiobumetanide Analogs

1. Thiobumetanide and dithiobumetanide

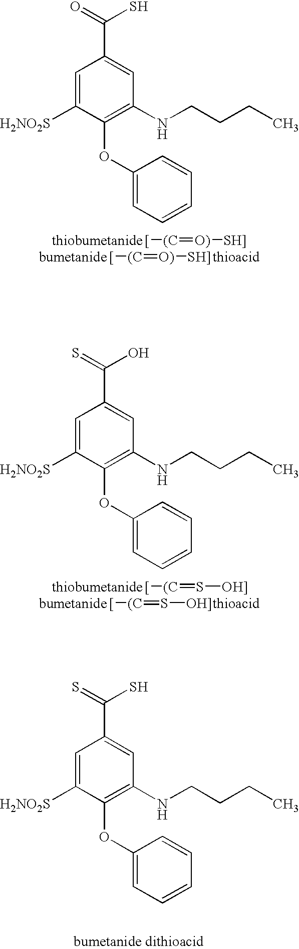

The thiobumetanide analogs are synthesized by reacting the carboxylic acid moiety of bumetanide with various reagents. For example, bumetanide may undergo conversion to the corresponding thioacid by treatment with thionyl chloride to form the corresponding bumetanide acid chloride followed by reaction with sodium hydrogen sulfide to give thiobumetanide[—(C═O)—SH], also known as bumetanide[—(C═O)—SH]thioacid by the methodology of Noble, P. and Tarbell, D. S., Org. Synth., Coll. Vol. IV, John Wiley & Sons, Inc., New York, 1963, 924-927. See Scheme 1. Thiobumetanide may undergo conversion to the corresponding bumetanide thioacid chloride with thionyl chloride, followed by treatment of the thioacid chloride with sodium hydrogen sulfide to give dithiobumetanide[—(C═S)—SH], also known as bumetanide[—(C═S)—SH]dithioacid by similar methodology. Reaction of bumetanide thioacid chloride with secondary amines will give the corresponding bumetanide thioamides. Bumetanide may also undergo reaction with phosphorous pentasulfide to yield bumetanide dithioacid. For reviews of this body of chemistry, see “Thioacyl Halides”, “Thiocarboxylic O-Acid Esters” and “Dithiocarboxylic Acid Esters”, all by Glass, R. S. in Science of Synthesis, (Charette, A. B., Ed.), Volume 22, Thieme Chemistry, 2005, Chapters 22.1.2, 22.1.3 and 22.1.4 and references therein. See also “Synthesis of Thioamides and Thiolactams”, Schaumaun, E., in Comprehensive Organic Synthesis, (Trost. B. M. and Fleming, I., Eds.), Permagon Press, 1991, Volume 6, Chapter 2.4, pp. 450-460 and references therein.

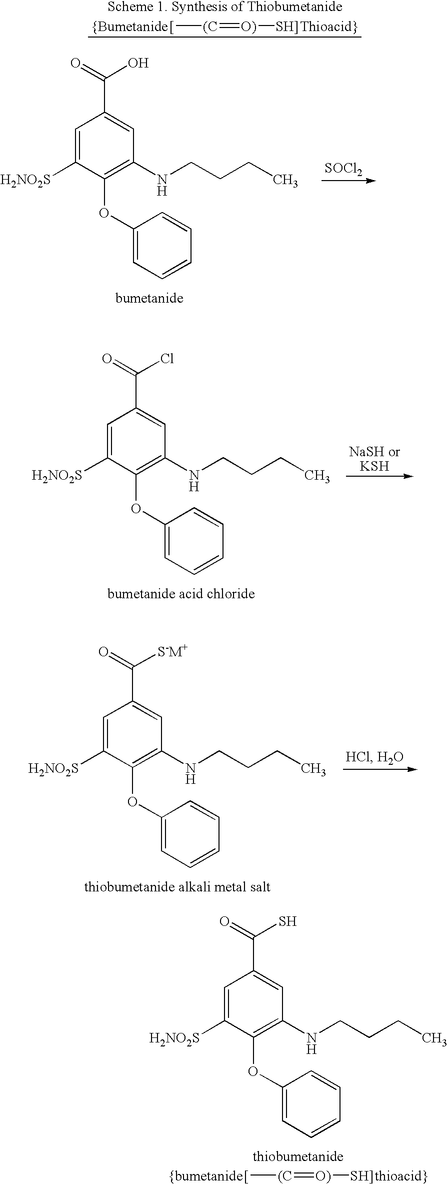

2. Bumetanide and S-Thiobumetanide Analogs

The bumetanide analogs are synthesized by reacting the carboxylic acid moiety of bumetanide with various reagents. For example, bumetanide may undergo esterification via reaction with alcohols, including linear, branched, substituted, or unsubstituted alcohols. Bumetanide or thiobumetanide may also be alkylated via reaction with suitable substituted and unsubstituted alkyl halides and alkaryl halides, including chloroacetonitrile, benzyl chloride, 1-(dimethylamino)propyl chloride. 2-chloro-N,N-diethylacetamide, and the like. PEG-type esters may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type esters may also be formed by alkylation by using alkyl halides such as chloromethyl pivalate or chloromethyl propionate. Bumetanide may also undergo amidation by reaction with suitable substituted or unsubstituted alkyl amines or aryl amines, either after conversion to the acid chloride or by using an activator, such as 1-ethyl-3-(3-dimethylaminopropyl)carbodiimide (EDC). Bumetanide or thiobumetanide may also be reacted with a quaternary ammonium hydroxide, such as benzyltrimethylammonium hydroxide or cetyltrimethylammonium hydroxide, to form bumetanide or thiobemetanide quaternary ammonium salts. Schemes 2, 3 and 4 present synthesis schemes of some exemplary compounds according to formula I.

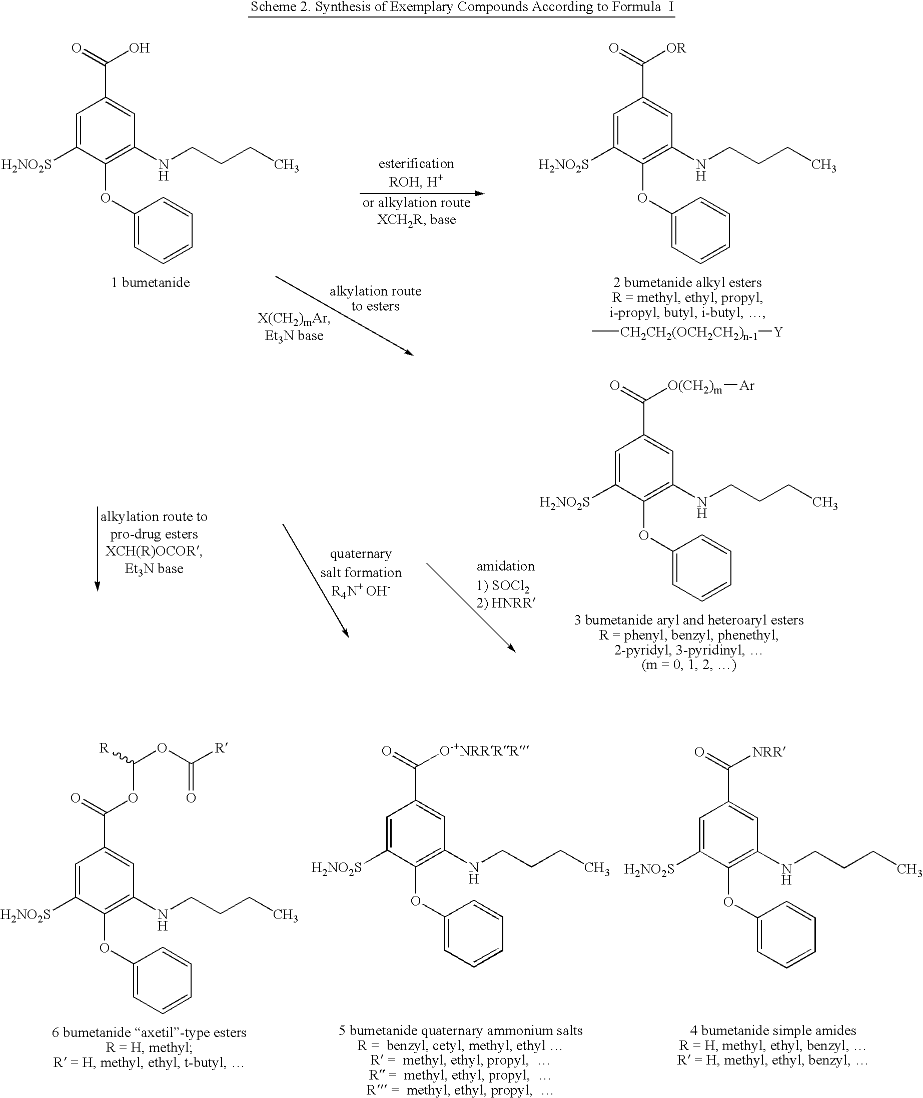

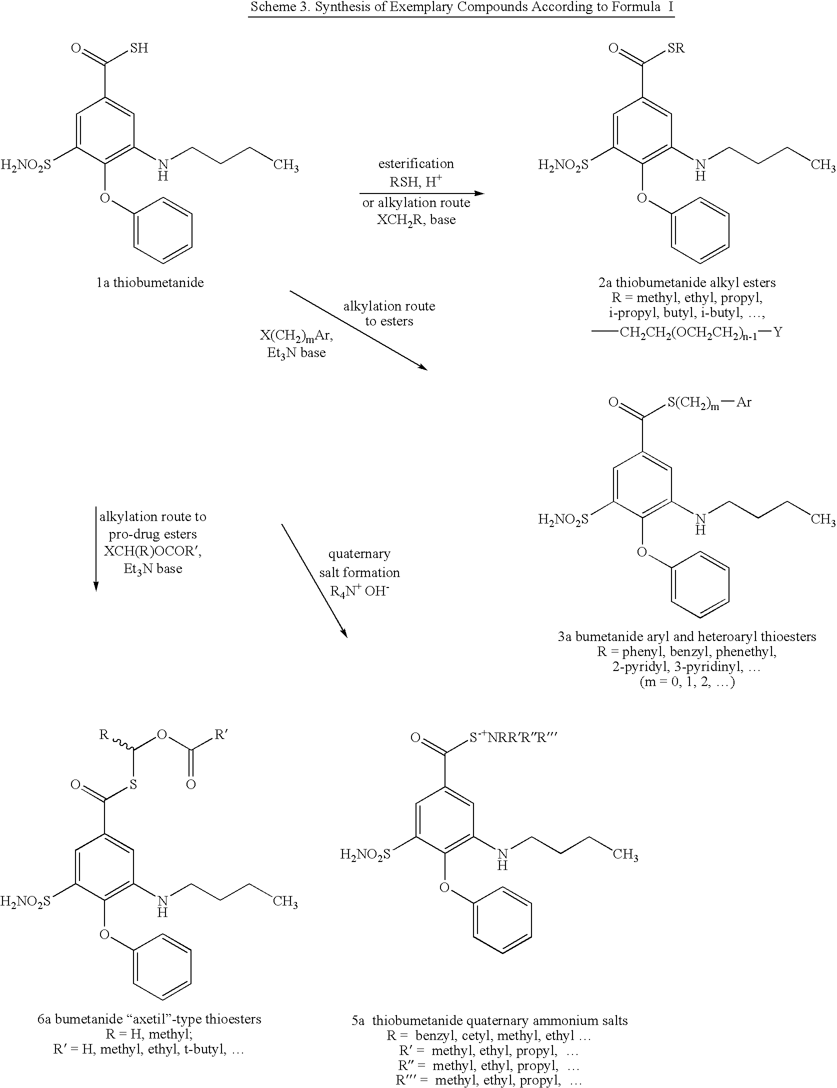

Bumetanide salts, thiobumetanide and S-thiobumetanide esters should readily undergo acid- and base-catalyzed hydrolysis to produce the carboxylic acid containing molecule bumetanide by methods well known in the art (See Yang, W. and Drueckhammer, D. G., J. Amer. Chem. Soc., 2001, 123 (44), 11004-11009 and references therein). (See Scheme 4).

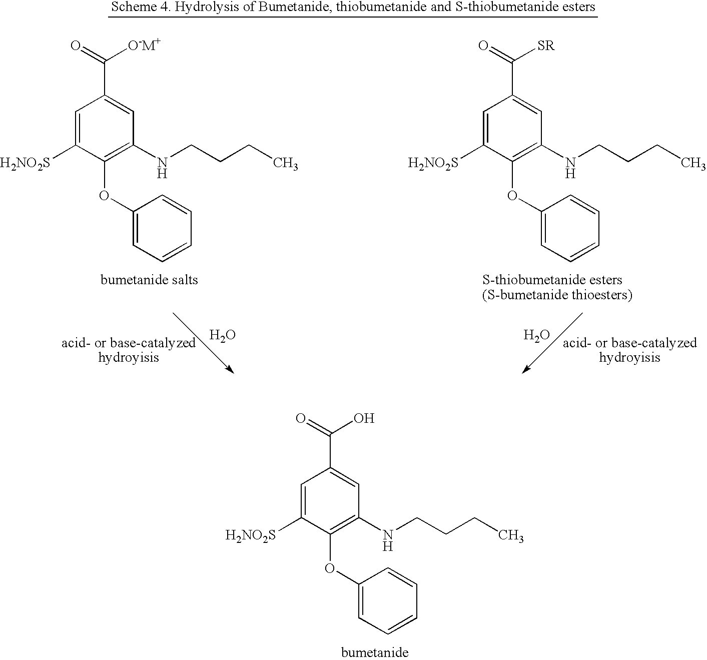

3. O-Substituted Thiobumetanide Analogs and Dithiobumetanide Analogs

Bumetanide may undergo conversion to the corresponding thioacid by treatment with thionyl chloride to form the corresponding acid chloride followed by reaction with sodium hydroxide or sodium hydrogen sulfide to give metastable O-thiobumetanide and dithiobumetanide by the methodology of Noble, P. and Tarbell, D. S., Org. Synth., Coll. Vol. IV, John Wiley & Sons. Inc., New York, 1963, 924-927. (See Schemes 5 and 6).

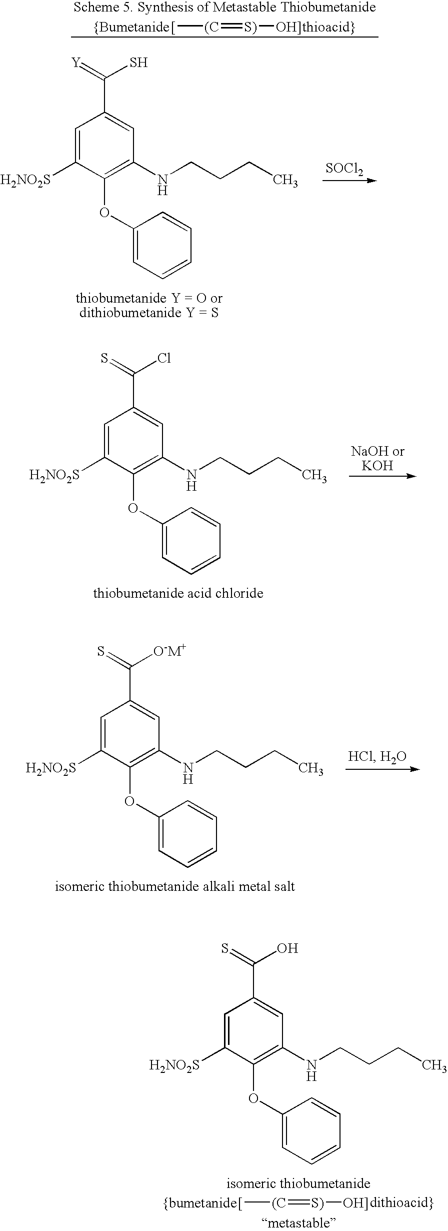

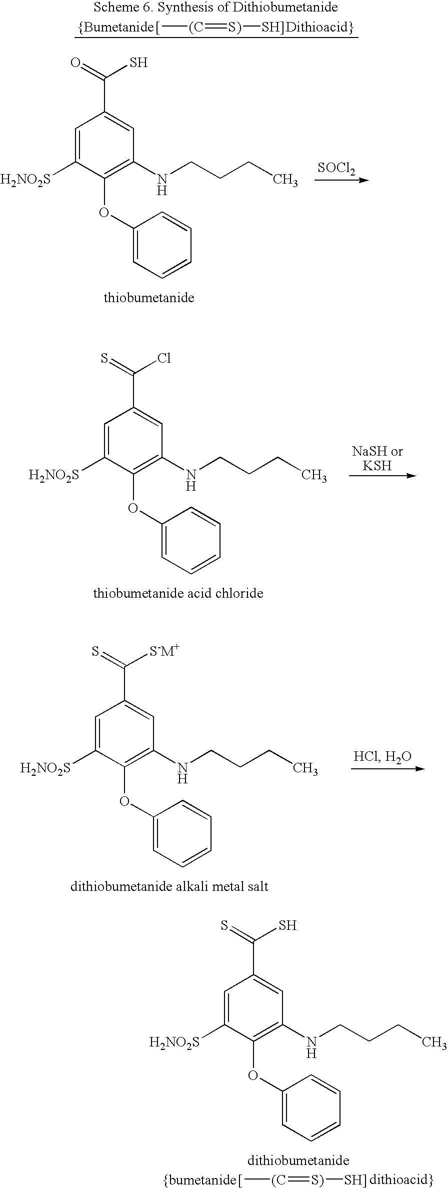

The thiobumetanide analogs are, in turn, synthesized by reacting the thiocarboxylic acid moiety of S-thiobumetanide with various reagents For example, S-thiobumetanide may undergo esterification via reaction with alcohols and thiols, including linear, branched, substituted, or unsubstituted alcohols and thiols. S-Thiobumetanide may also be alkylated via reaction with suitable substituted and unsubstituted alkyl halides and alkaryl halides, including chloroacetonitrile, benzyl chloride, 1-(dimethylamino)propyl chloride, 2-chloro-N,N-diethylacetamide, and the like. PEG-type esters may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type esters may also be formed by alkylation by using alkyl halides such as chloromethyl pivalate or chloromethyl propionate. S-Thiobumetanide may also be reacted with a quaternary ammonium hydroxide, such as benzyltrimethylammonium hydroxide or cetyltrimethylammonium hydroxide, to form thiobumetanide quaternary ammonium salts. See Schemes 7, 8 and 9, which present some exemplary compounds according to formula II.

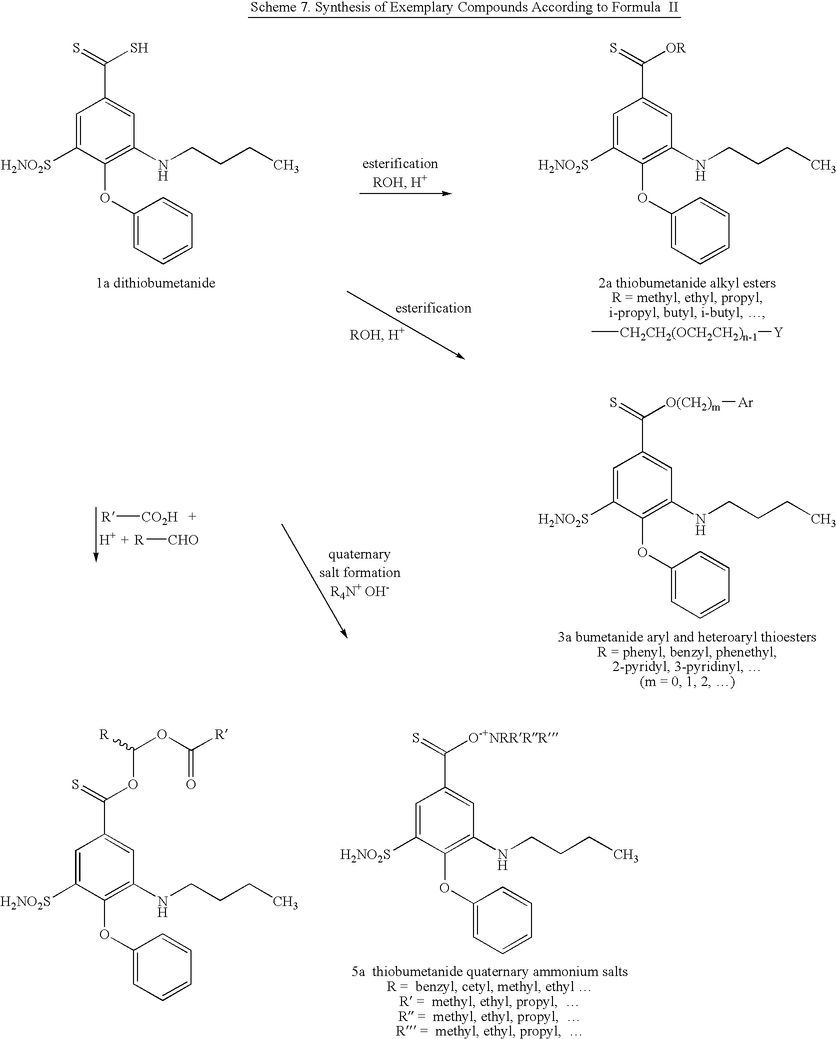

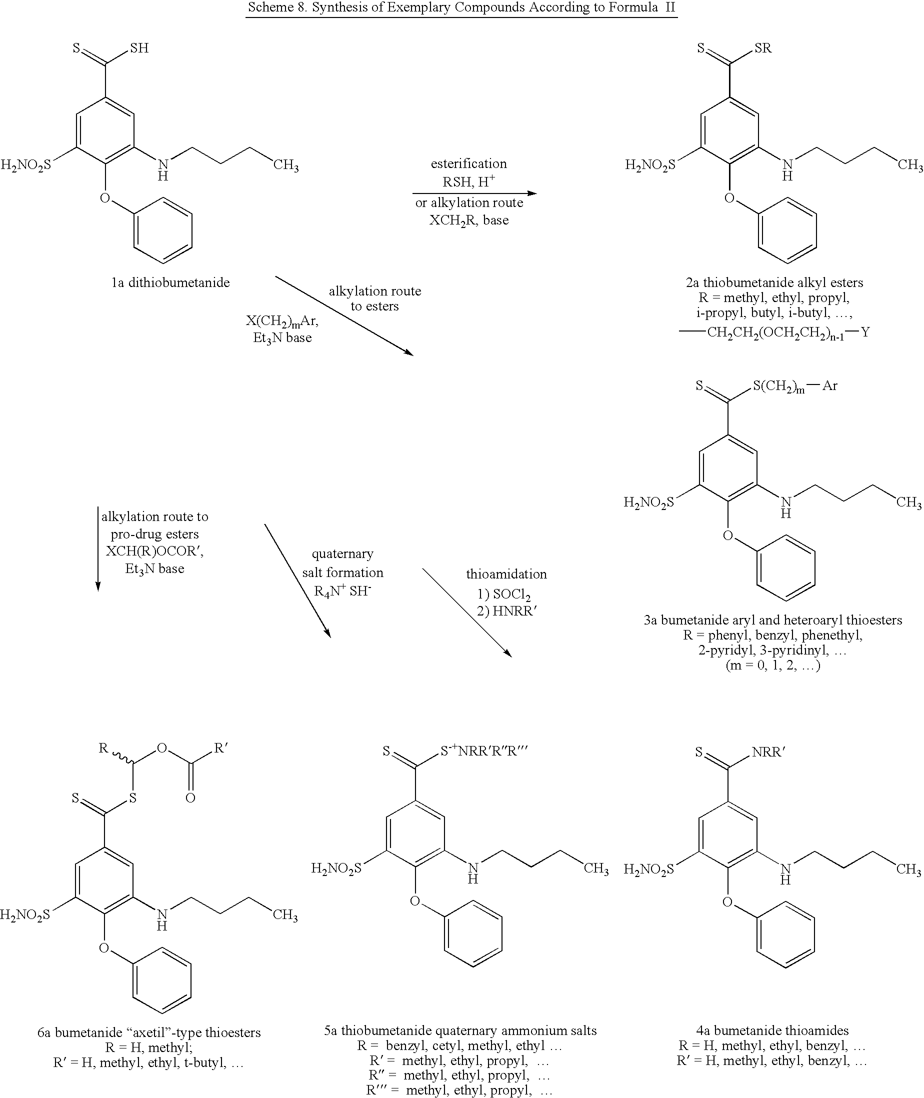

Thiobumetanide, thiobumetanide amides, O-thiobumetanide esters and dithiobumetanide esters should readily undergo acid- and base-catalyzed hydrolysis to produce the carboxylic acid containing molecule bumetanide by methods well known in the art (See Yang, W. and Drueckhammer, D. G., J. Amer. Chem. Soc., 2001, 123 (44), 11004-11009 and references therein). For additional reviews of this body of chemistry, see “Thioacyl Halides”, “Thiocarboxylic O-Acid Esters” and “Dithiocarboxylic Acid Esters”, all by Glass, R. S. in Science of Synthesis, (Charette, A. B., Ed.), Volume 22, Thieme Chemistry, 2005, Chapters 22.1.2, 22.1.3 and 22.1.4 and references therein. See also “Synthesis of Thioamides and Thiolactams”. Schaumann, E., in Comprehensive Organic Synthesis, (Trost, B. M. and Fleming, I., Eds.), Permagon Press, 1991, Volume 6, Chapter 2.4, pp. 450-460 and references therein. (See Scheme 9). 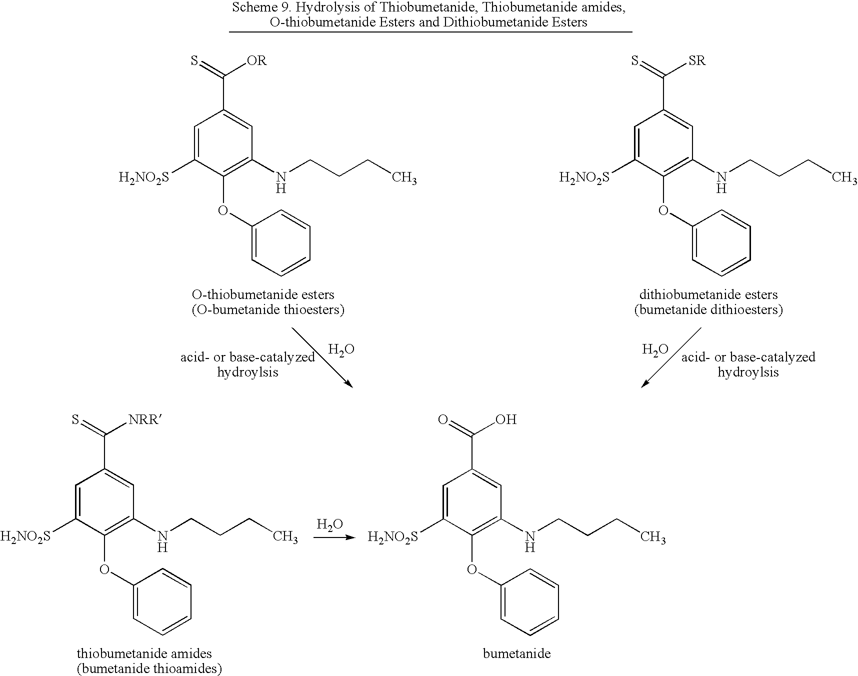
B. Furosemide Analogs, Thiofurosemide Analogs and Dithiofurosemide Analogs

1. Thiofurosemide and Dithiofurosemide

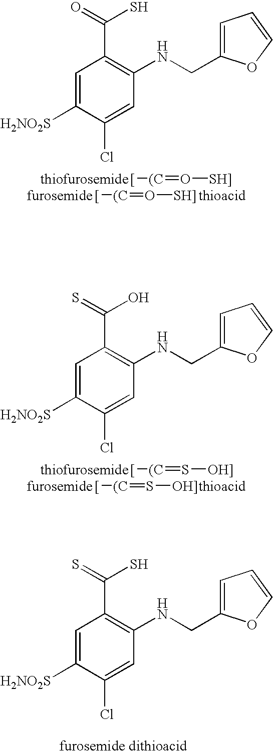

The thiofurosemide analogs are synthesized by reacting the carboxylic acid moiety of furosemide with various reagents. For example, furosemide may undergo conversion to the corresponding thioacid by treatment with thionyl chloride to form the corresponding furosemide acid chloride followed by reaction with sodium hydrogen sulfide to give thiofurosemide[—(C═O)—SH], also known as furosemide[—(C═O)—SH]thioacid by the methodology of Noble, P. and Tarbell, D. S., Org. Synth., Coll. Vol. IV, John Wiley & Sons, Inc., New York, 1963, 924-927. (See Scheme 10).

Thiofurosemide may undergo conversion to the corresponding furosemide thioacid chloride with thionyl chloride, followed by treatment of the thioacid chloride with sodium hydrogen sulfide to give dithiofurosemide[—(C═S)—SH], also known as furosemide[—(C═S)—SH]dithioacid by similar methodology. (See Scheme 10) Reaction of furosemide thioacid chloride with secondary amines will give the corresponding furosemide thioamides. Furosemide may also undergo reaction with phosphorous pentasulfide to yield furosemide dithioacid. For reviews of this body of chemistry, see “Tiioacyl Halides”, “Thiocarboxylic O-Acid Esters” and “Dithiocarboxylic Acid Esters”, all by Glass, R. S. in Science of Synthesis, (Charette, A. B., Ed.), Volume 22, Thieme Chemistry. 2005, Chapters 22.1.2, 22.1.3 and 22.1.4 and references therein. See also “Synthesis of Thioamides and Thiolactams”, Schaumann, E., in Comprehensive Organic Synthesis, (Trost, B. M. and Fleming. I., Eds.), Permagon Press, 1991, Volume 6, Chapter 2.4, pp. 450-460 and references therein.

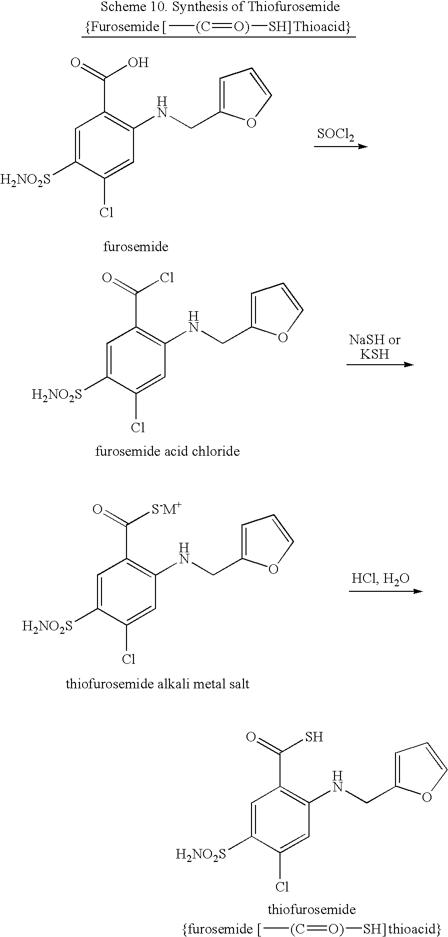

2. Furosemide and S-Furosemide Analogs

The furosemide analogs are synthesized by methods analogous to those used in the synthesis of the bumetanide analogs. Furosemide may undergo esterification via reaction with alcohols, including linear, branched, substituted, or unsubstituted alcohols. Furosemide or thiofurosemide may also be alkylated via reaction with suitable substituted and unsubstituted alkyl halides and alkaryl halides, including for example, chloroacetonitrile, benzyl chloride, 1-(dimethylamino)propyl chloride, 2-chloro-N,N-diethylacetamide, and the like. PEG-type esters may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type esters may also be formed by alkylation by using alkyl halides such as chloromethyl pivalate or chloromethyl propionate. Furosemide may also undergo amidation by reaction with suitable substituted or unsubstituted alkyl amines or aryl amines, either after conversion to the acid chloride or by using an activator, such as 1-ethyl-3-(3-dimethylaminopropyl)carbodiimide (ELDC). Furosemide or thiofurosemide may also be reacted with a quaternary ammonium hydroxide, such as benzyltrimethylammonium hydroxide or cetyltrimethylammonium hydroxide, to form furosemide or thiofurosemide quaternary ammonium salts. Schemes, 11, 12 and 13 present some exemplary compounds according to formula III.

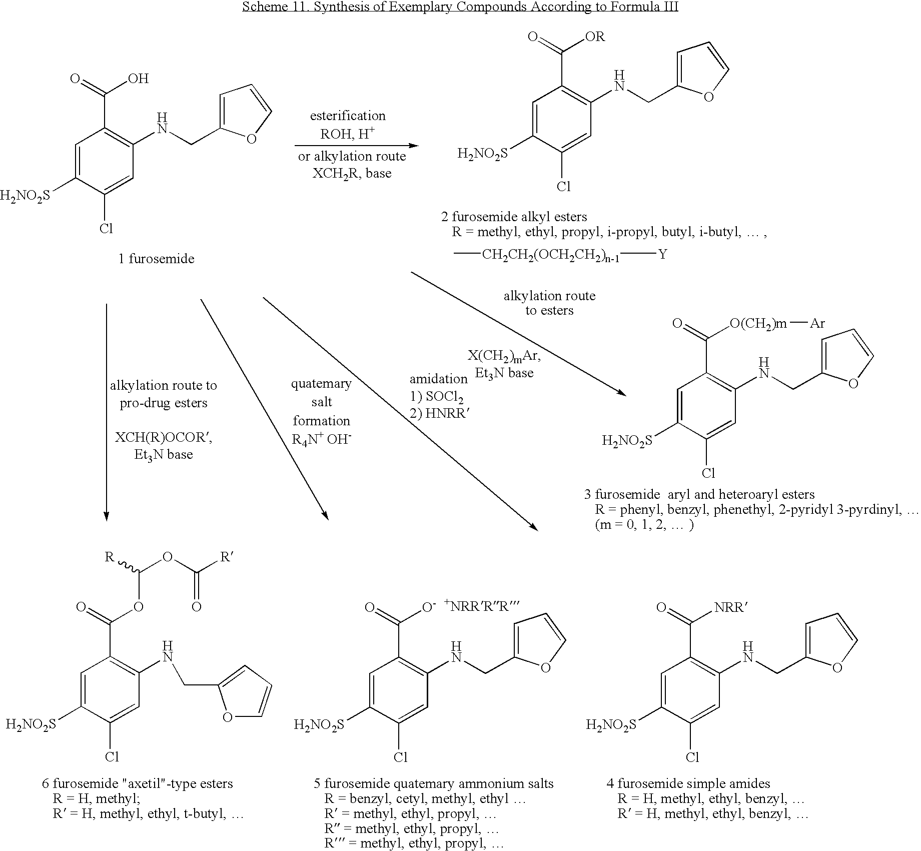

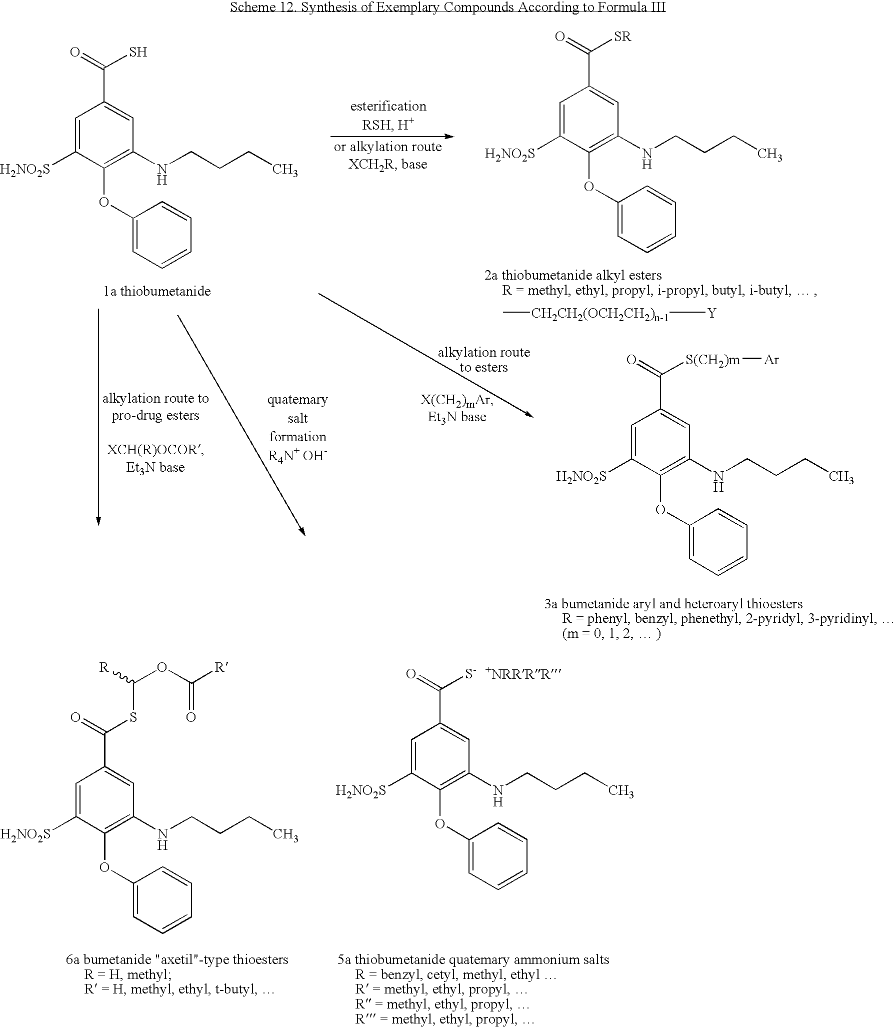

Thiofurosemide salts and S-thiofurosemide esters should readily undergo acid- and base-catalyzed hydrolysis to produce the carboxylic acid containing molecule furosemide by methods well known in the art (See Yang, W. and Drueckhammer, D. G., J. Amer. Chem. Soc., 2001, 123 (44), 11004-11009 and references therein). (See Scheme 13).

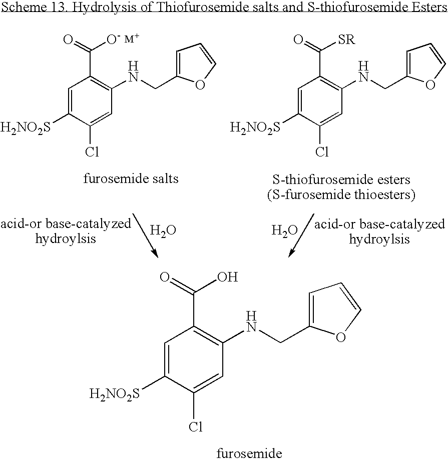

3. O-Substituted Thiofurosemide and Dithiofurosemide Analogs

Furosemide may undergo conversion to the corresponding thioacid by treatment with thionyl chloride to form the corresponding acid chloride followed by reaction with sodium hydroxide or sodium hydrogen sulfide to give O-thiofurosemide and dithiofurosemide by the methodology of Noble, P. and Tarbell. D. S., Org. Synth., Coll. Vol. IV, Joln Wiley & Sons, Inc., New York, 1963, 924-927. (See Schemes 14 and 15).

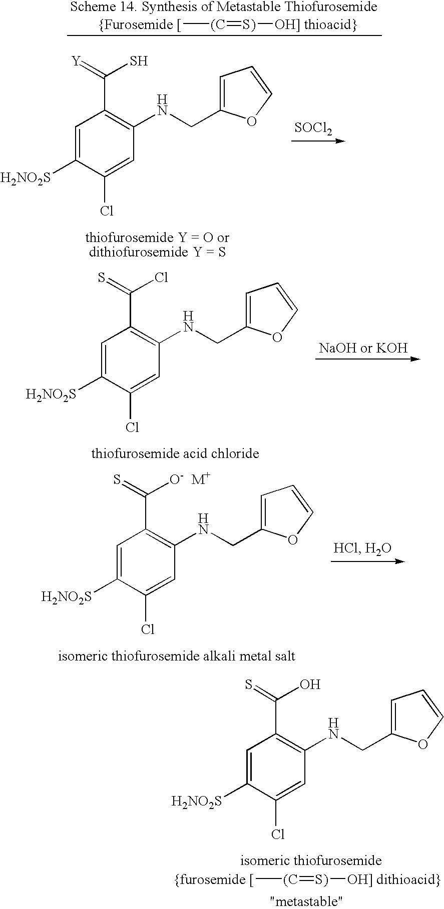

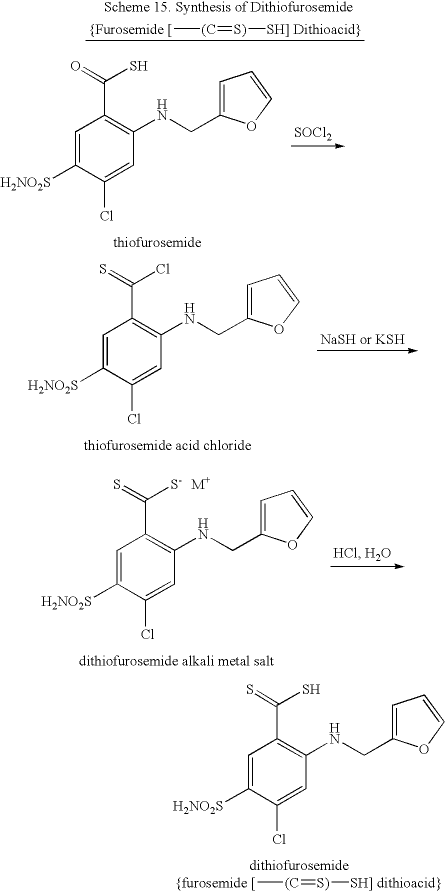

The thiofurosemide analogs are, in turn, synthesized by reacting the thiocarboxylic acid moiety of thiofurosemide with various reagents. For example, thiofurosemide may undergo esterification via reaction with alcohols or thiols, including linear, branched, substituted, or unsubstituted alcohols and thiols. S-Thiofurosemide may also be alkylated via reaction with suitable substituted and unsubstituted alkyl halides and alkaryl halides, including chloroacetonitrile, benzyl chloride, 1-(dimethylamino)propyl chloride, 2-chloro-N,N-diethylacetamide, and the like. PEG-type esters may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type esters may also be formed by alkylation by using alkyl halides such as chloromethyl pivalate or chloromethyl propionate. Thiofurosemide may also be reacted with a quaternary ammonium hydroxide, such as benzyltrimethylammonium hydroxide or cetyltrimethylammonium hydroxide, to form thiofurosemide quaternary ammonium salts. Schemes 14, 15, 16, 17 and 18 present synthesis schemes of some exemplary compounds according to formula IV.

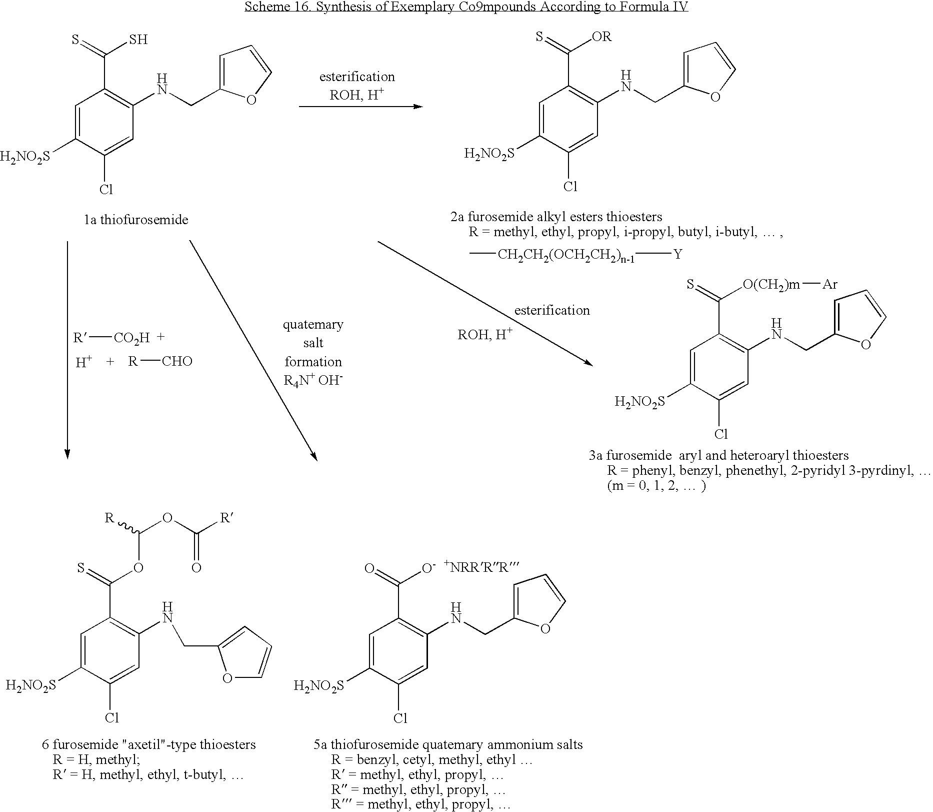

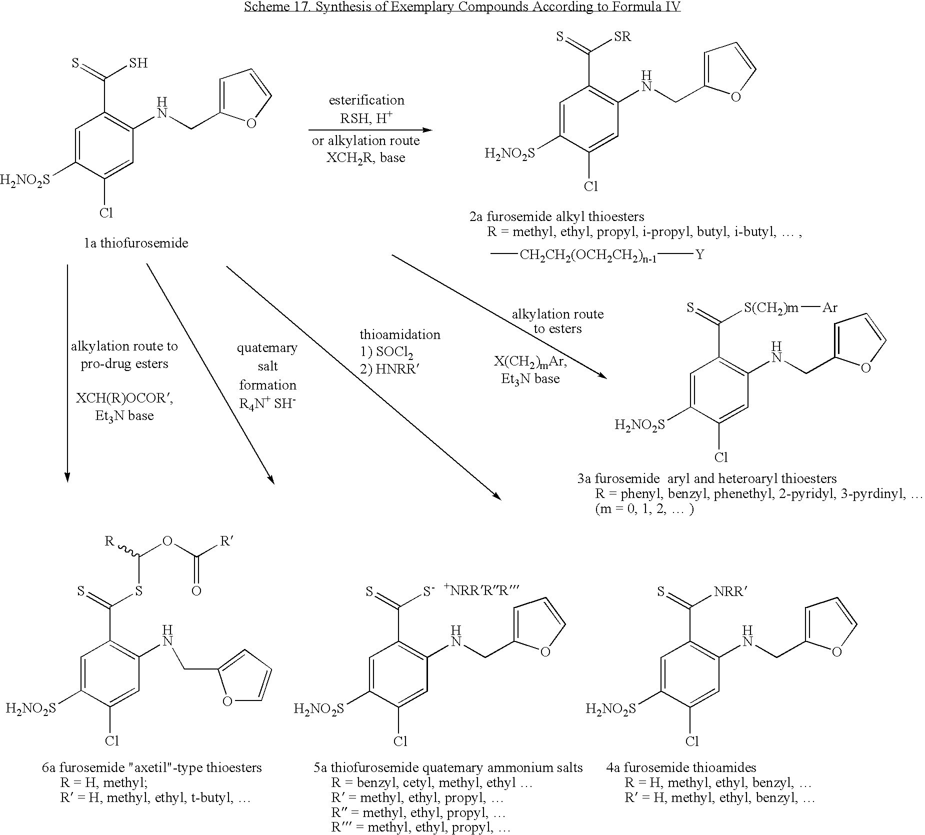

Thiofurosemide, thiofurosemide amides and S-thiofurosemide esters should readily undergo acid- and base-catalyzed hydrolysis to produce the carboxylic acid containing molecule furosemide by methods well known in the art (See Yang, W. and Drueckhammer, D. G., J. Amer. Chem. Soc., 2001, 123 (44), 11004-11009 and references therein). For additional reviews of this body of chemistry, see “Thioacyl Halides”. “Thiocarboxylic O-Acid Esters” and “Dithiocarboxylic Acid Esters”, all by Glass, R. S. in Science of Synthesis, (Charette, A. B., Ed.), Volume 22, Thieme Chemistry, 2005, Chapters 22.1.2, 22.1.3 and 22.1.4 and references therein. See also “Synthesis of Thioamides and Thiolactams”, Schaumann, E., in Comprehensive Organic Synthesis. (Trost, B. M. and Fleming, I., Eds.), Permagon Press, 1991, Volume 6, Chapter 2.4, pp. 450-460 and references therein. (See Scheme 18). 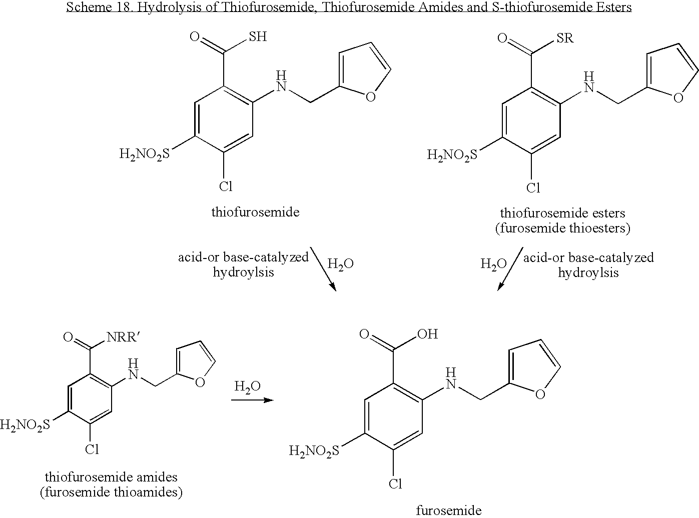
C. Piretanide Analogs, Thiopiretanide Analogs and Dithiopiretanide Analogs

1. Thiopiretanide and Dithiopiretanide

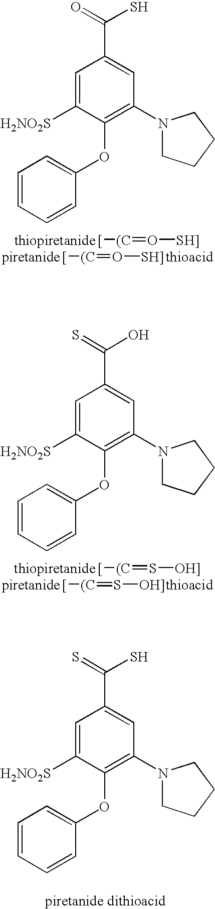

The piretanide analogs are synthesized by reacting the carboxylic acid moiety of piretanide with various reagents. For example, piretanide may undergo conversion to the corresponding thioacid by treatment with thionyl chloride to form the corresponding piretanide acid chloride followed by reaction with sodium hydrogen sulfide to give thiopiretanide[—(C═O)—SH], also known as piretanide[—(C═O)—SH]thioacid by the methodology of Noble, P. and Tarbell, D. S., Org. Synth., Coll. Vol. IV, John Wiley & Sons, Inc., New York, 1963, 924-927. See Scheme 19. Thiopiretanide may undergo conversion to the corresponding piretanide thioacid chloride with thionyl chloride, followed by treatment of the thioacid chloride with sodium hydrogen sulfide to give dithiopiretanide[—(C═S)—SH], also known as piretanide[—(C═S)—SH]dithioacid by similar methodology. Reaction of piretanide thioacid chloride with secondary amines will give the corresponding piretanide thioamides. Piretanide may also undergo reaction with phosphorous pentasulfide to yield piretanide dithioacid. For reviews of this body of chemistry, see “Thioacyl Halides”, “Thiocarboxylic O-Acid Esters” and “Dithiocarboxylic Acid Esters”, all by Glass, R. S. in Science of Synthesis. (Charette, A. B., Ed.), Volume 22. Thieme Chemistry, 2005, Chapters 22.1.2, 22.1.3 and 22.1.4 and references therein. See also “Synthesis of Thioamides and Thiolactams”, Schaumann, E., in Comprehensive Organic Synthesis, (Trost, B. M. and Fleming, I., Eds.), Pernagon Press, 1991, Volume 6, Chapter 2.4, pp. 450-460 and references therein.

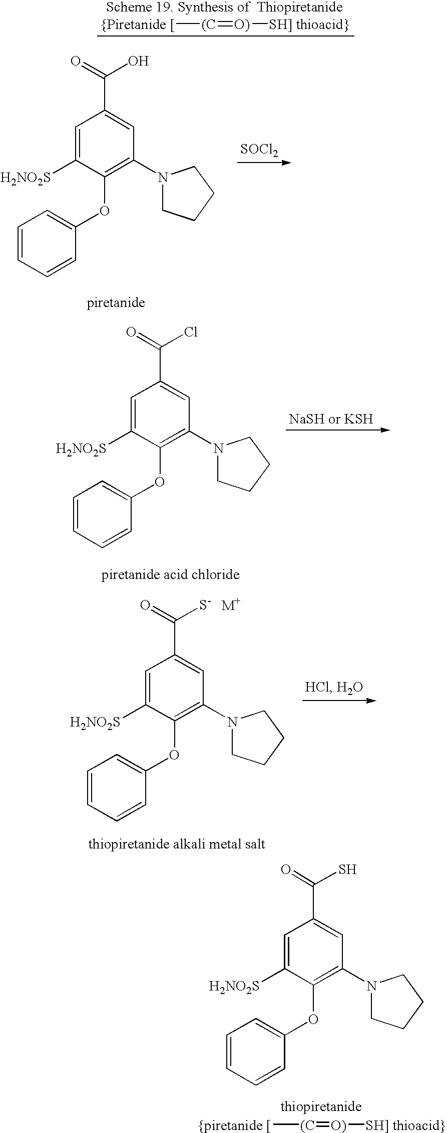

2. Piretanide and S-Thiopiretanide Analogs

Piretanide may undergo esterification via reaction with alcohols, including linear, branched, substituted, or unsubstituted alcohols. Piretanide or thiopiretanide may also be alkylated via reaction with suitable substituted and unsubstituted alkyl halides and alkaryl halides, including chloroacetonitrile, benzyl chloride, 1-(dimethylamino)propyl chloride, 2-chloro-N,N-diethylacetamide, and the like. PEG-type esters may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type esters may also be formed by alkylation by using alkyl halides such as chloromethyl pivalate or chloromethyl propionate. Piretanide may also undergo amidation by reaction with suitable substituted or unsubstituted alkyl amines or aryl amines, either after conversion to the acid chloride or by using an activator, such as 1-ethyl-3-(3-dimethylaminopropyl)carbodiimide (EDC). Piretanide or thiopiretanide may also be reacted with a quaternary ammonium hydroxide, such as benzyltrimethylammonium hydroxide or cetyltrimethylammonium hydroxide, to form piretanide or thiopiretanide quaternary ammonium salts. Schemes 19, 20, 21 and 22 present synthesis schemes of some exemplary compounds according to formula V.

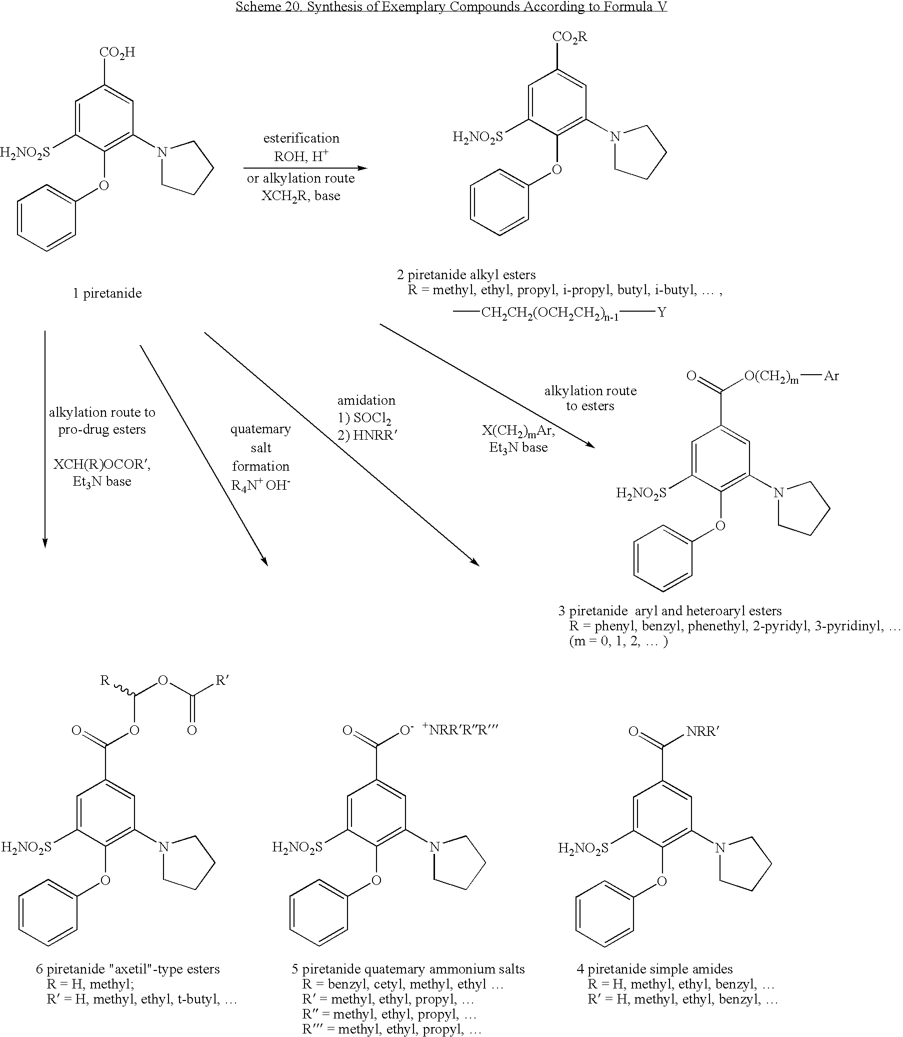

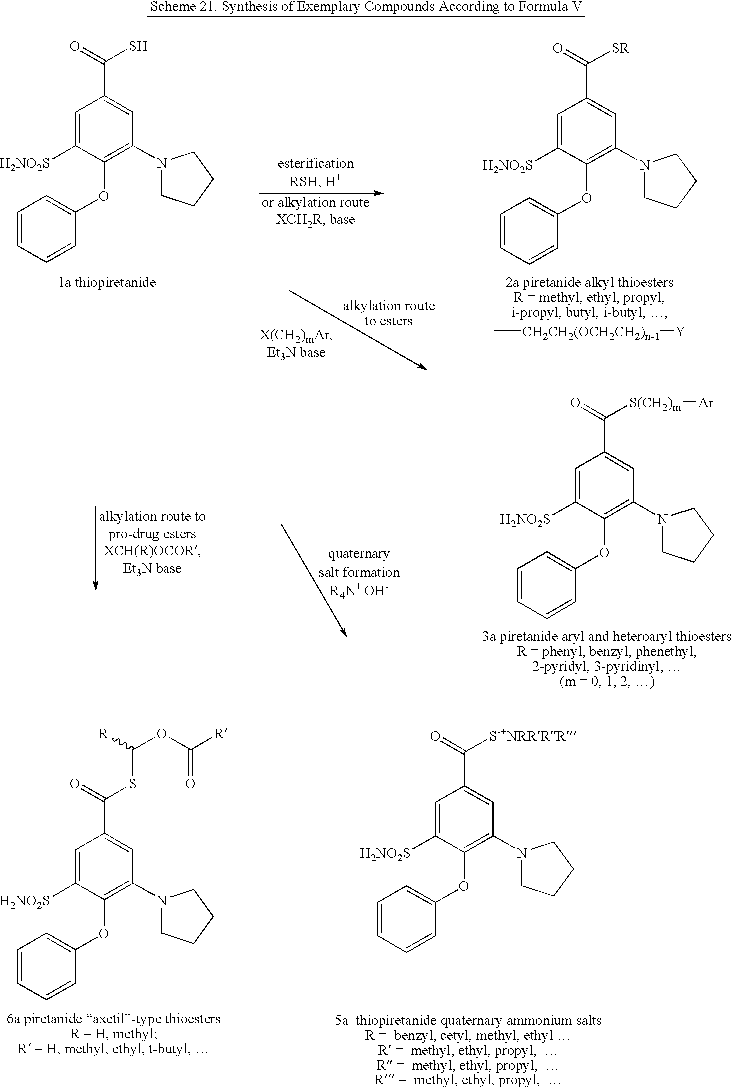

Thiopiretanide salts and S-thiopiretanide esters should readily undergo acid- and base-catalyzed hydrolysis to produce the carboxylic acid containing molecule bumetanide by methods well known in the art (See Yang, W., Drueckhammer D. G., J. Amer. Chem. Soc., 2001, 123 (44), 11004-11009 and references therein). (See Scheme 22).

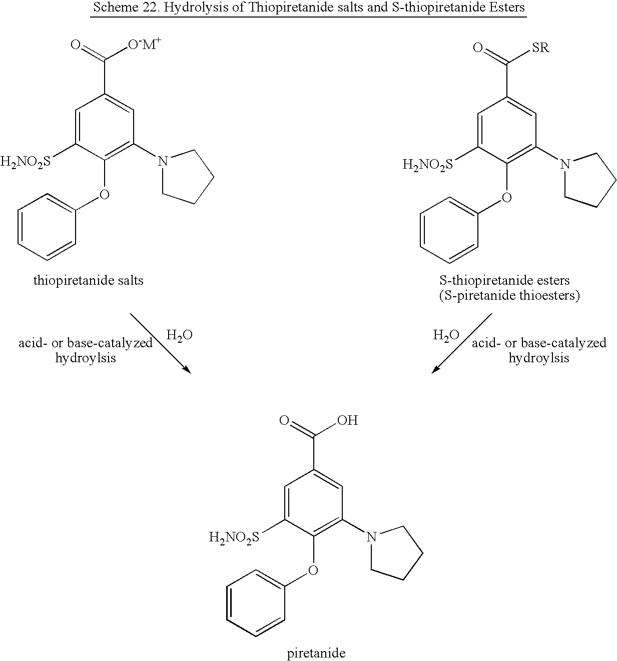

3. O-Substituted Thiopiretanide Analogs Dithiopireatanide Analogs

Piretanide may undergo conversion to the corresponding thioacid by treatment with thionyl chloride to form the corresponding acid chloride followed by reaction with sodium hydroxide or sodium hydrogen sulfide to give metastable O-thiopiretanide and dithiopiretanide by the methodology of Noble, P. and Tarbell, D. S., Org. Synth., Coll. Vol. IV, John Wiley & Sons, Inc., New York, 1963, 924-927. See Schemes 23 and 24.

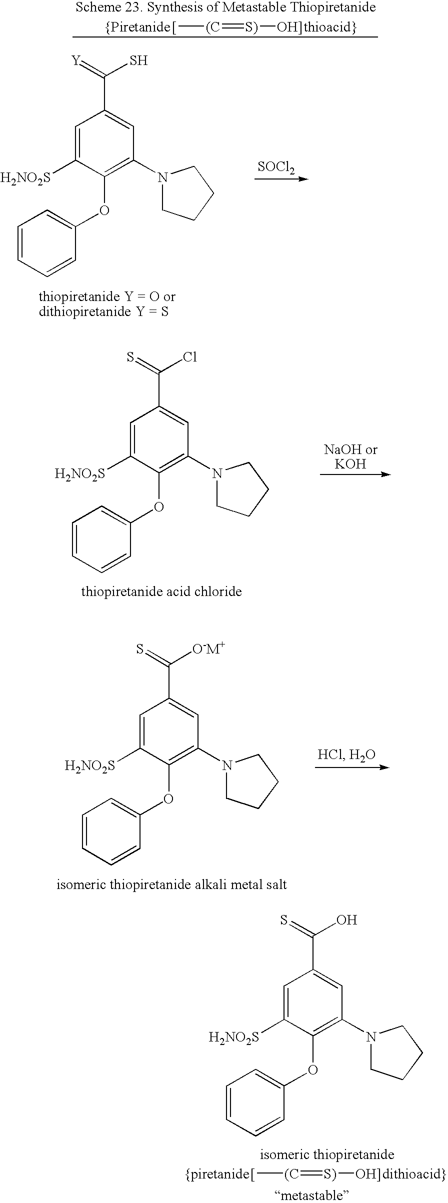

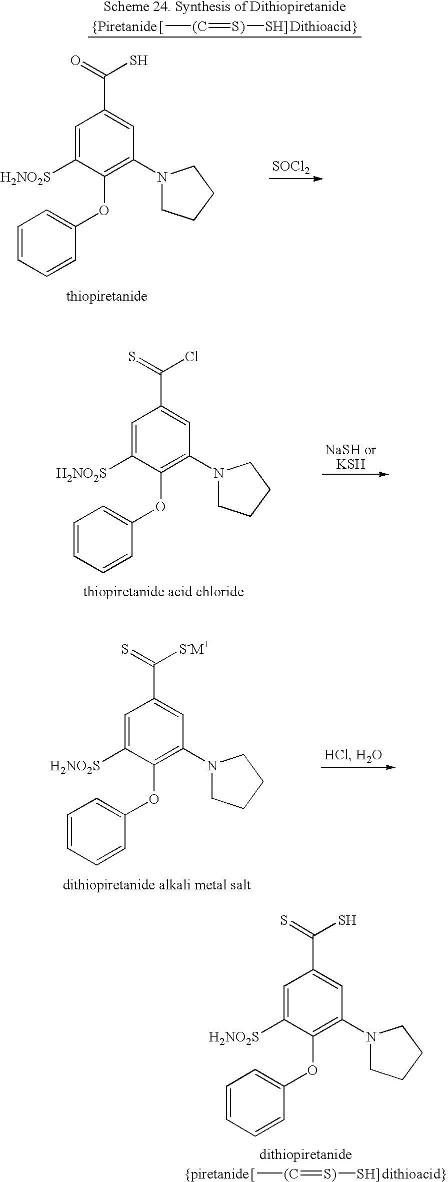

The thiopiretanide analogs are synthesized by methods analogous to those used in the synthesis of the piretanide analogs. Specifically, thiopiretanide may undergo esterification via reaction with thiols, including linear, branched, substituted, or unsubstituted thiols, Thiopiretanide may also be alkylated via reaction with suitable substituted and unsubstituted alkyl halides and alkaryl halides, including chloroacetonitrile, benzyl chloride, 1-(dimethylamino)propyl chloride, 2-chloro-N,N-diethylacetamide, and the like. PEG-type esters may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type thioesters may also be formed by alkylation by using alkyl halides such as chloromethyl pivalate or chloromethyl propionate. Thiopiretanide may also be reacted with a quaternary ammonium hydroxide, such as benzyltrimethylammonium hydroxide or cetyltrimethylammonium hydroxide, to form thiopiretanide quaternary ammonium salts. Schemes 23, 24, 25, 26 and 27 present some exemplary compounds according to formula VI.

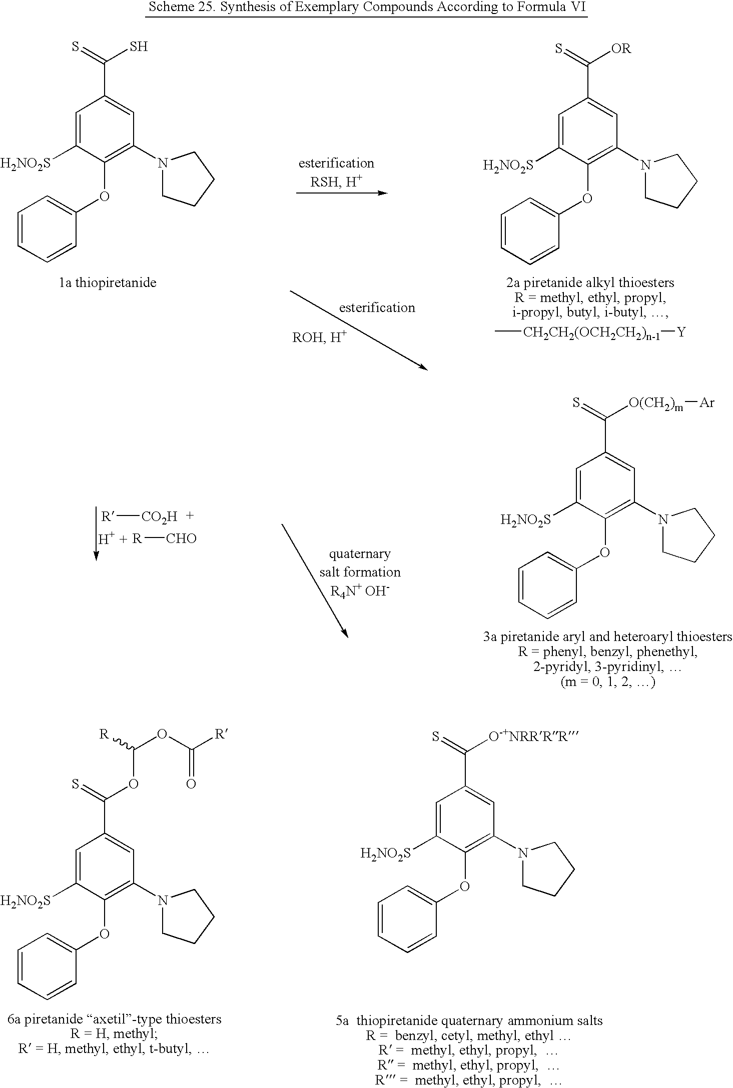

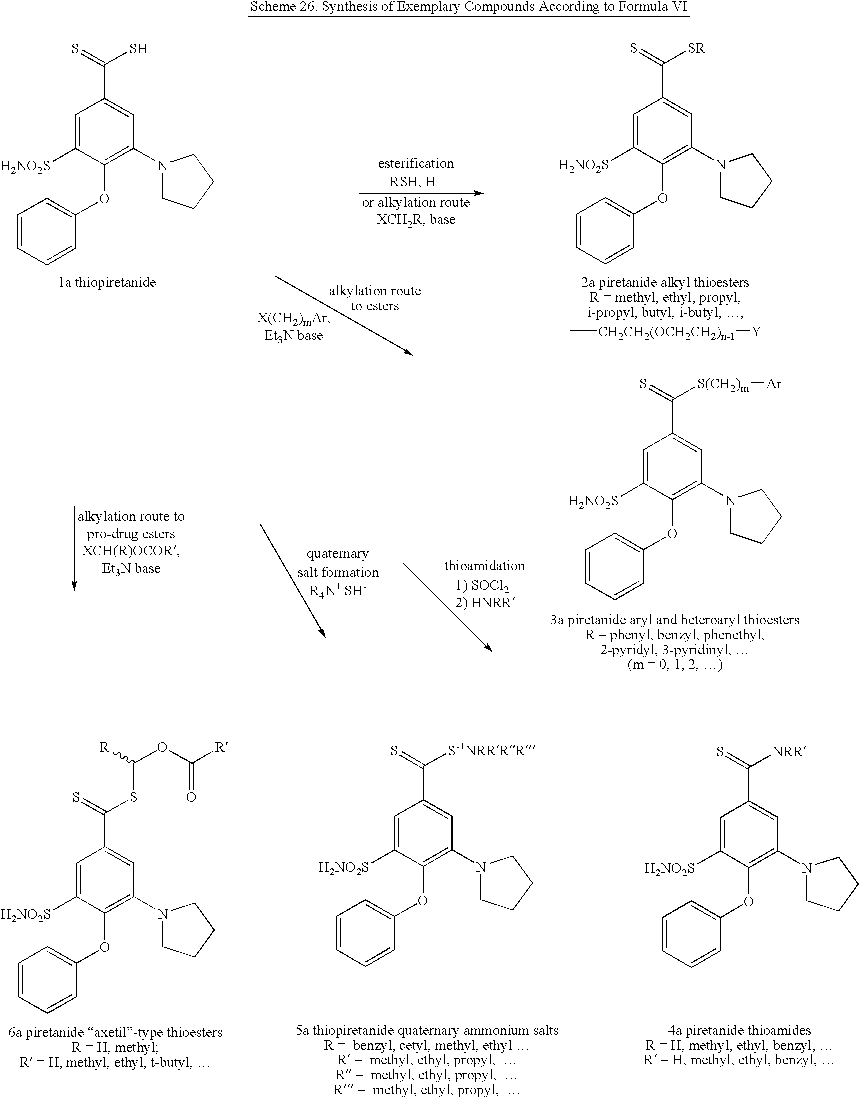

Thiopiretanide, thiopiretanide amides and thiopiretanide esters should readily undergo acid- and base-catalyzed hydrolysis to produce the carboxylic acid containing molecule piretanide by methods well known in the art (See Yang, W. and Drueckhammer, D. G., J. Amer. Chem. Soc., 2001, 123 (44)7 11004-11009 and references therein). For additional reviews of this body of chemistry, see “Thioacyl Halides”, “Thiocarboxylic O-Acid Esters” and “Dithiocarboxylic Acid Esters”, all by Glass, R. S. in Science of Synthesis, (Charette. A. B., Ed.), Volume 22, Thieme Chemistry, 2005, Chapters 22.1.2, 22.1.3 and 22.1.4 and references therein. See also “Synthesis of Thioamides and Thiolactams”, Schaumann, E., in Comprehensive Organic Synthesis. (Trost, B. M. and Fleming, I., Eds.), Permagon Press, 1991, Volume 6, Chapter 2.4, pp. 450-460 and references therein. (See Scheme 27). 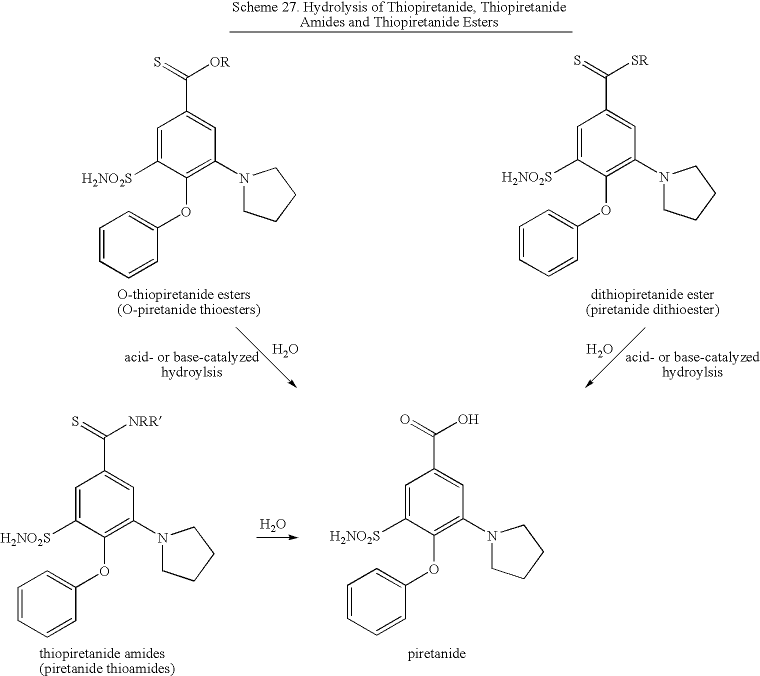
D. Azosemide Analogs

The azosemide analogs are synthesized by the reaction of various reagents with the tetrazolyl moiety of azosemide. Azosemide may undergo hydroxyalkylation with the addition of an aldehyde, whereby a hydroxylalkyl functionality is formed. An alcohol may optionally be reacted along with the aldehyde to obtain an ether. An alkyl thiol may optionally be added with the aldehyde to form a thioether. Azosemide may also be alkylated by the addition of suitable alkyl halides or alkaryl halides, including alkyl or alkaryl halides comprising an ether or thioether linkage, such as methyl chloromethyl ether and benzyl chloromethyl thioether. PEG-type ethers may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type analogs may also be formed via addition of alkyl or alkaryl halides, such as chloromethyl pivalate or chloromethyl propionate. Azosemide may also be reacted with a quaternary ammonium salt, such as benzyltrimethylammonium and base such as sodium hydroxide or cetyltrimethylammonium bromide and base such as sodium hydroxide, in order to form an azosemide quaternary ammonium salt. Scheme 28 below presents a synthesis scheme of some exemplary compounds according to formula VII. 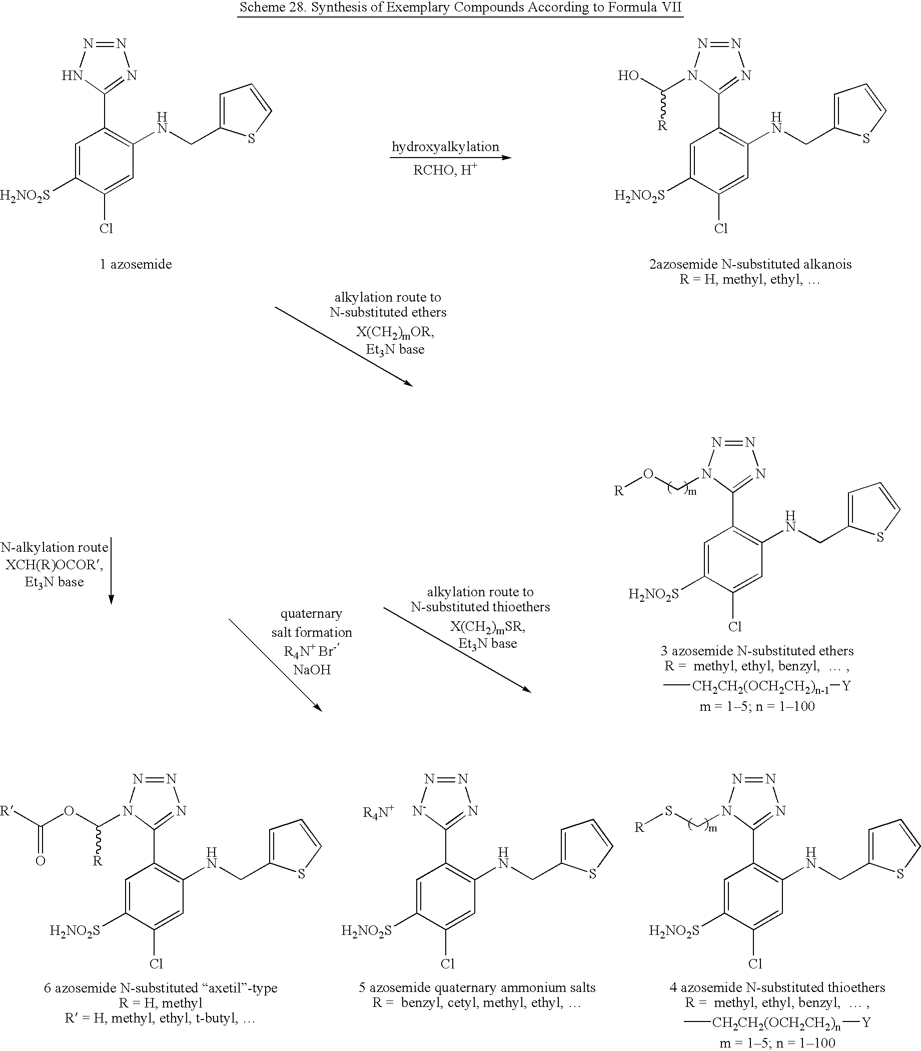
E. Torsemide Analogs

The torsemide (also known as torasemide) analogs are synthesized by the reaction of various reagents with the pyridine moiety of torsemide. Torsemide may undergo alkylation by the addition of suitable alkyl or alkaryl halides, including benzyl chloride, to form N-substituted quaternary ammonium salts. Alkyl halides and alkaryl halides comprising an ether linkage, including methyl chloromethyl ether and benzyl chloromethyl ether, may be used to form N-substituted ether quaternary ammonium salts. Alkyl halides and alkaryl halides comprising a thioether linkage, including methyl chloromethyl thioether and benzyl chloromethyl thioether, may be used to form N-substituted thioether quaternary ammonium salts. PEG-type ether-containing quaternary ammonium salts may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. “Axetil”-type quaternary ammonium salts may also be formed via the addition of alkyl halides such as chloromethyl pivalate or chloromethyl propionate. Scheme 29 below presents a synthesis scheme of some exemplary compounds according to formula VIII. 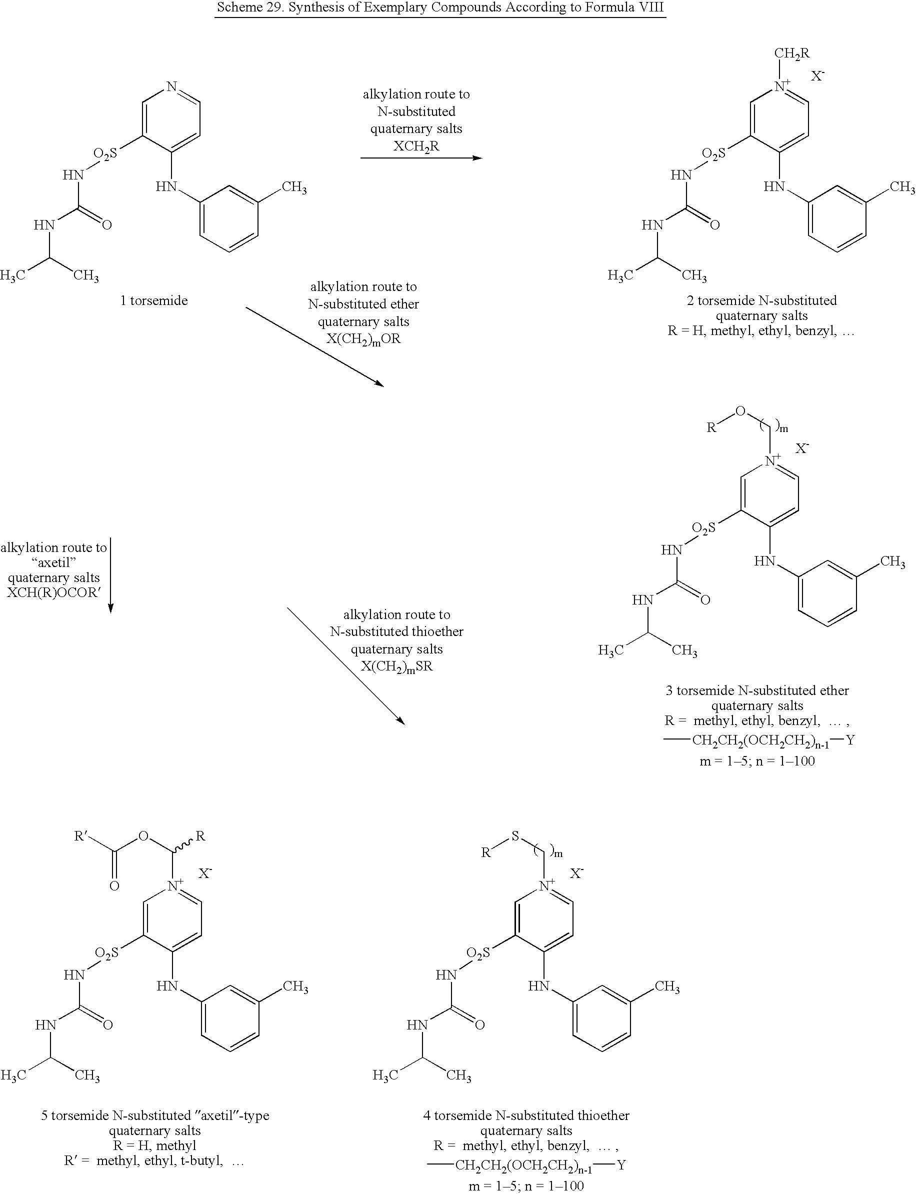
F. Benzaldehyde Analogs of Bumetanide, Piretanide and Furosemide

The substituted benzoic acids bumetanide, piretanide and furosemide can be selectively reduced to the corresponding bumetanide aldehyde, piretanide aldehyde and furosemide aldehyde using amine-substituted ammonium hydrides such as bis(4-methylpiperazinyl)aluminum hydride by literature methods. See Muraki. M. and Mukiayama, T., Chem. Letters, 1974, 1447; Muraki, M. and Mukiayama, T., Chem. Letters, 1975, 215; and Hubert, T., D., Eyman, D. P. and Wiemer, D. F., J. Org. Chem., 1984, 2279. (See Scheme 30) It is well known that the more lipophilic benzaldehydes readily air-oxidize into the more hydrophilic benzoic acids and that benzaldehydes are also metabolized into the corresponding benzoic acids in vivo, via the use of NADPH co-factor and with a number of oxidative P450 enzymes.

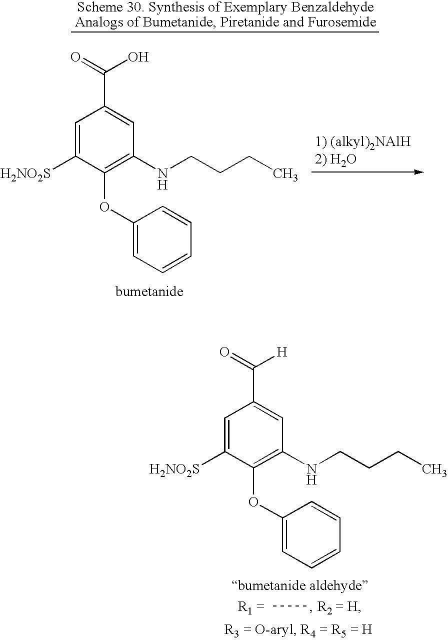

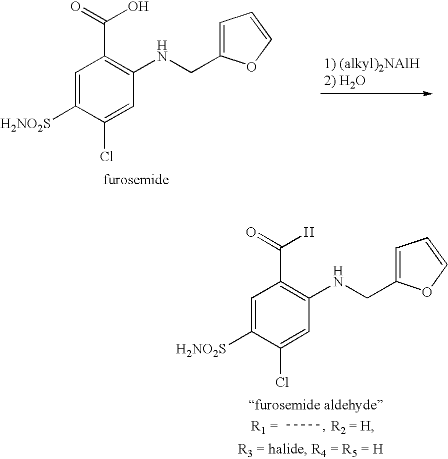

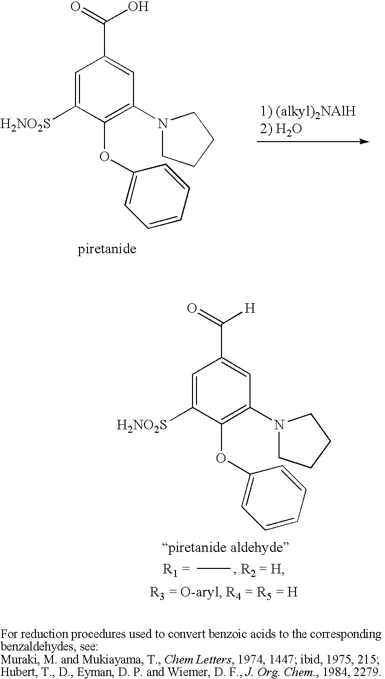

The lipophilic thiobenzaldehydes can also be prepared from the corresponding benzaldehydes by treating agents including hydrogen sulfide and diphosphorus pentasulfide (See Smith, M. B. and March, J., March's Advanced Organic Chemistry, Fifth Edition, 2001, John Wiley & Sons, Inc., New York, Part 2, Chapter 16, pp. 1185-1186. C. Sulfur Nucleophiles, Section 16-10 The Addition of H₂S and Thiols to Carbonyl Compounds.) (See Scheme 31). In turn these thiobenzaldehydes are readily converted back into the corresponding benzaldehydes under hydrolytic conditions. It is well known that the more lipophilic benzaldehydes readily air-oxidize into the more hydrophilic benzoic acids and that benzaldehydes are also metabolized into the corresponding benzoic acids in vivo, via the use of NADPH co-factor and with a number of oxidative P450 enzymes. A similar mechanism can be applied for the conversions of thiobenzaldehydes to thiobenzoic acids and then benzoic acids. 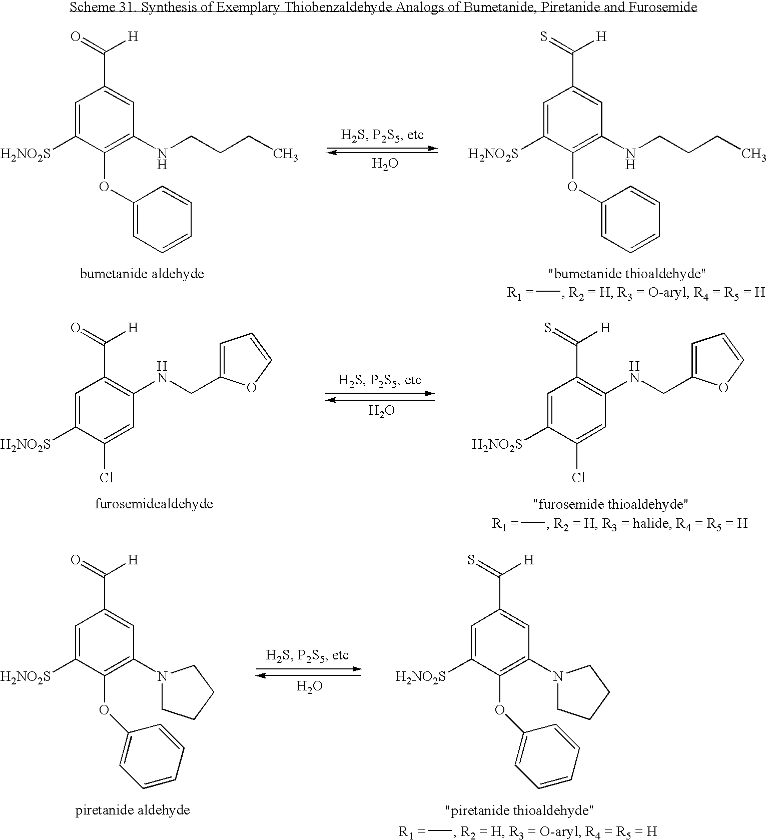
G. PEG-Type Analogs of Bumetanide, Piretanide and Furosemide and their Thioacid Counterparts Thiobumetanide, Thiopiretanide, Thiofurosemide Dithiobumetanide, Dithiopiretanide and Dithiofurosemide

The PEG-type esters of bumetanide, furosemide and piretanide may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. (See Scheme 32).

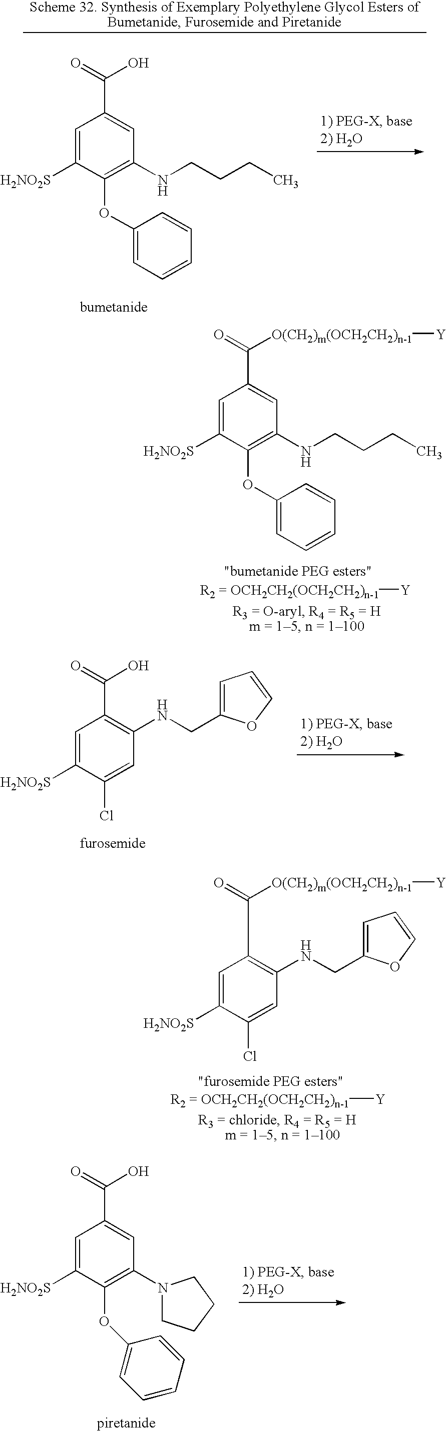

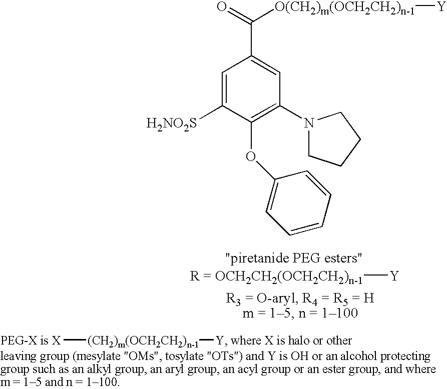

The PEG-type esters of thiobumetanide, thiofurosemide and thiopiretanide may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. (See Scheme 33).

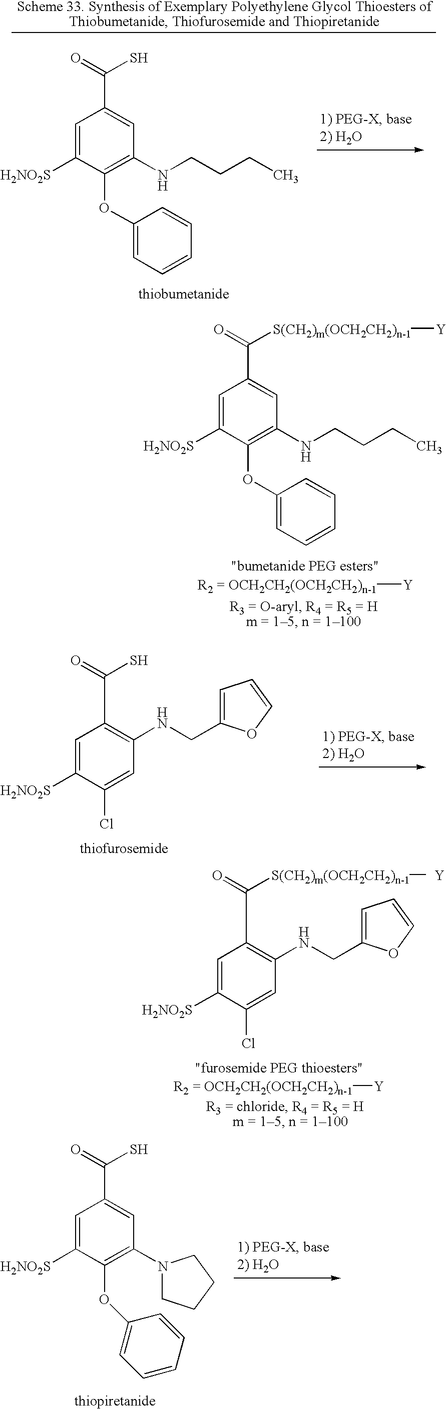

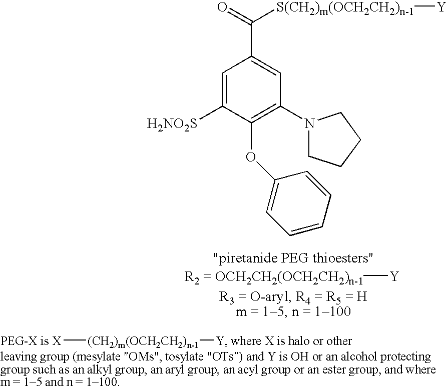

The PEG-type esters of dithiobumetanide, dithiofurosemide and dithiopiretanide may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. (See Scheme 34). 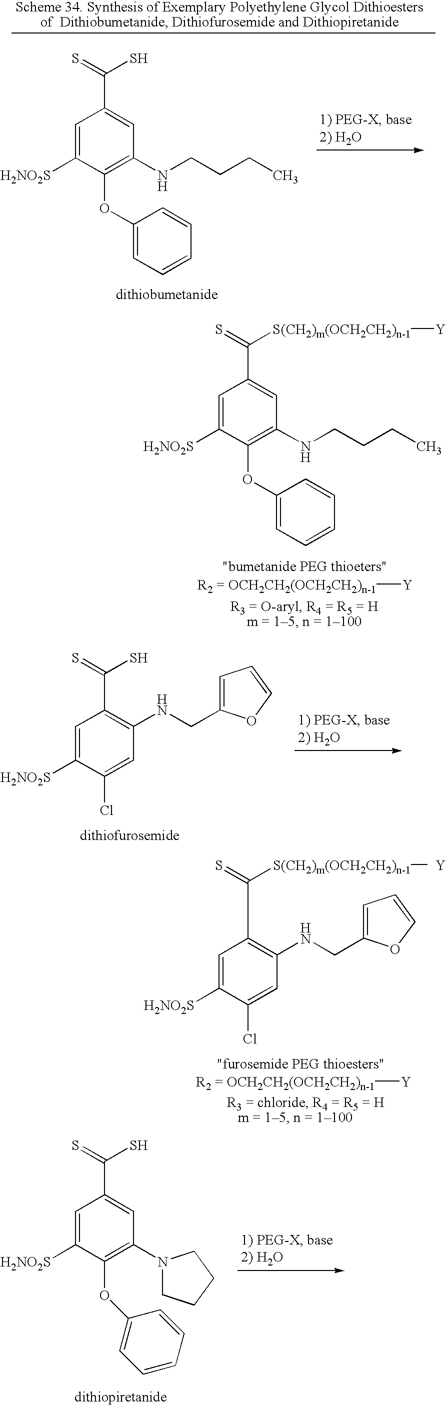 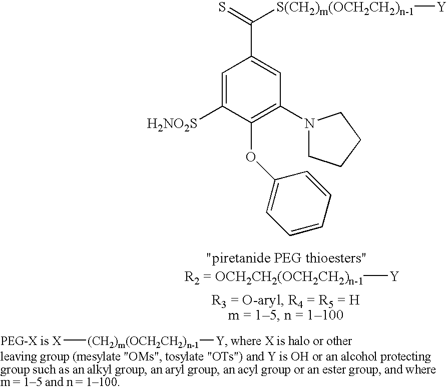
H. PEG-Type Analogs of Azosemide and Torsemide

The PEG-type ethers of azosemide and torsemide may be formed by alkylation using alkyloxy(polyalkyloxy)alkyl halides such as MeO-PEG350-Cl and the like or alkyloxy(polyalkyloxy)alkyl tosylates such as MeO-PEG1000-OTs and the like. (See Scheme 35).

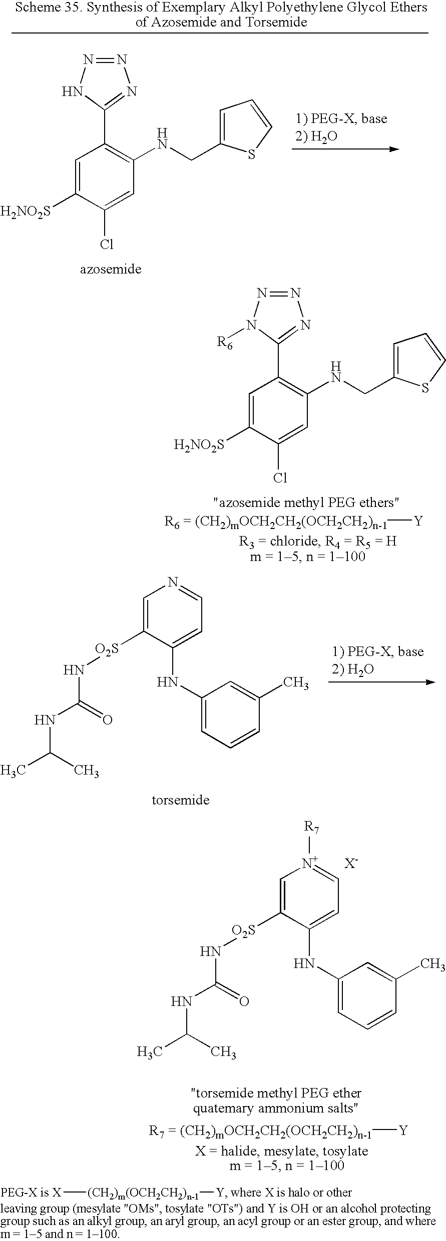

Starting materials for synthesizing compounds of the present invention can further include compounds described in U.S. Pat. No. 3,634,583 to Feit; U.S. Pat. No. 3,806,534 to Fiet; U.S. Pat. No. 3,058,882 to Struem et al.; U.S. Pat. No. 4,010,273 to Bormarir; U.S. Pat. No. 3,665,002 to Popelak; and U.S. Pat. No. 3,665,002 to Delarge.

Compounds of the present invention can include isomers, tautomers, zwitterions, enantiomers, diastereomers, racemates or stereochemical mixtures thereof. The term “isomers” as used herein refers to compounds having the same number and kind of atoms, and hence the same molecular weight, but differing with respect to the arrangement or configuration of the atoms in space. Additionally, the term “isomers” includes stereoisomers and geometric isomers. The terms “stereoisomer” or “optical isomer” as used herein refer to a stable isomer that has at least one chiral atom or restricted rotation giving rise to perpendicular dissymmetric planes (e.g., certain biphenyls, allenes, and spiro compounds) and can rotate plane-polarized light. Because asymmetric centers and other chemical structure can exist in some of the compounds of the present invention which may give rise to stereoisomerism, the invention contemplates stereoisomers and mixtures thereof. The compounds of the present invention and their salts can include asymmetric carbon atoms and may therefore exist as single stereoisomers, racemates, and as mixtures of enantiomers and diastereomers. Typically, such compounds will be prepared as a racemic mixture. If desired, however, such compounds can be prepared or isolated as pure stereoisomers, i.e., as individual enantiomers or diastereomers, or as stereoisomer-enriched mixtures. Tautomers are readily interconvertible constitutional isomers and there is a change in connectivity of a ligand, as in the keto and enol forms of ethyl acetoacetate (The present invention includes tautomers of any said compounds.) Zwitterions are inner salts or dipolar compounds possessing acidic and basic groups in the same molecule. At neutral pH, the cation and anion of most zwitterions are equally ionized.

3. Pharmaceutical Compositions

The compounds of the present invention or pharmacologically acceptable salts thereof may be formulated into pharmaceutical compositions of various dosage forms. To prepare the pharmaceutical compositions of the invention, one or more compounds, or pharmaceutically acceptable salts thereof as the active ingredient is intimately mixed with appropriate carriers and additives according to techniques well known to those skilled in the art of pharmaceutical formulations.

A pharmaceutically acceptable salt as used herein refers to a salt form of a compound permitting its use or formulation as a pharmaceutical and which retains the biological effectiveness of the free acid and base of the specified compound and that is not biologically or otherwise undesirable. Examples of such salts are described in Handbook of Pharmaceutical Salts: Properties, Selection, and Use, Wermuth, C. G. and Stahl, P. H. (eds.), Wiley-Verlag Helvetica Acta, Zurich, 2002 [ISBN 3-906390-26-8]. Examples of such salts include alkali metal salts and addition salts of free acids and bases. Examples of pharmaceutically acceptable salts, without limitation, include sulfates, pyrosulfates, bisulfates, sulfites, bisulfites, phosphates, monohydrogenphosphates, dihydrogen phosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, xylenesulfonates, phenylacetates, phenylpropionates, phenylbutyrates, citrates, lactates, γ-hydroxybutyrates, glycollates, tartrates, methanesulfonates, ethane sulfonates, propanesulfonates, toluenesulfonates, naphthalene-1-sulfonates, naphthalene-2-sulfonates, and mandelates. In some embodiments, pharmaceutically acceptable salt includes sodium, potassium, calcium, ammonium, trialkylarylammonium and tetraalkylammonium salts.

The carriers and additives used for such pharmaceutical compositions can take a variety of forms depending on the anticipated mode of administration. Thus, compositions for oral administration may be, for example, solid preparations such as tablets, sugar-coated tablets, hard capsules, soft capsules, granules, powders and the like, with suitable carriers and additives being starches, sugars, binders, diluents, granulating agents, lubricants, disintegrating agents and the like, Because of their ease of use and higher patient compliance, tablets and capsules represent advantageous oral dosage forms for many medical conditions.

Similarly, compositions for liquid preparations include solutions, emulsions, dispersions, suspensions, syrups, elixirs, and the like with suitable carriers and additives being water, alcohols, oils, glycols, preservatives, flavoring agents, coloring agents, suspending agents, and the like. Typical preparations for parenteral administration comprise the active ingredient with a carrier such as sterile water or parenterally acceptable oil including polyethylene glycol, polyvinyl pyrrolidone, lecithin, arachis oil or sesame oil, with other additives for aiding solubility or preservation may also be included. In the case of a solution, it can be lyophilized to a powder and then reconstituted immediately prior to use. For dispersions and suspensions, appropriate carriers and additives include aqueous gums, celluloses, silicates or oils.

The pharmaceutical compositions according to embodiments of the present invention include those suitable for oral, rectal, topical, nasal, inhalation (e.g., via an aerosol) buccal (e.g., sub-lingual), vaginal, topical (i.e., both skin and mucosal surfaces, including airway surfaces), transdermal administration and parenteral (e.g., subcutaneous, intramuscular, intradermal, intraarticular, intrapleural, intraperitoneal, intrathecal, intracerebral, intracranially, intraarterial, or intravenous), although the most suitable route in any given case will depend on the nature and severity of the condition being treated and on the nature of the particular active agent which is being used. Pharmaceutical compositions of the present invention are particularly suitable for oral, sublingual, parenteral, implantation, nasal and inhalational administration.

Compositions for injection will include the active ingredient together with suitable carriers including organic solvents, propylene glycol-alcohol-water, isotonic water, sterile water for injection (USP), emulPhor™-alcohol-water, cremophor-EL™ or other suitable carriers known to those skilled in the art. These carriers may be used alone or in combination with other conventional solubilizing agents such as ethanol, a glycol, or other agents known to those skilled in the art.

Where the compounds of the present invention are to be applied in the form of solutions or injections, the compounds may be used by dissolving or suspending in any conventional diluent. The diluents may include, for example, physiological saline, Ringer's solution, an aqueous glucose solution, an aqueous dextrose solution, an alcohol, a fatty acid ester, glycerol, a glycol, an oil derived from plant or animal sources, a paraffin and the like. These preparations may be prepared according to any conventional method known to those skilled in the art.

Compositions for nasal administration may be formulated as aerosols, drops, powders and gels. Aerosol formulations typically comprise a solution or fine suspension of the active ingredient in a physiologically acceptable aqueous or non-aqueous solvent. Such formulations are typically presented in single or multidose quantities in a sterile form in a sealed container. The sealed container can be a cartridge or refill for use with an atomizing device. Alternatively, the sealed container may be a unitary dispensing device such as a single use nasal inhaler, pump atomizer or an aerosol dispenser fitted with a metering valve set to deliver a therapeutically effective amount, which is intended for disposal once the contents have been completely used. When the dosage form comprises an aerosol dispenser, it will contain a propellant such as a compressed gas, air as an example, or an organic propellant including a fluorochlorohydrocarbon or fluorohydrocarbon.

Compositions suitable for buccal or sublingual administration include tablets, lozenges and pastilles, wherein the active ingredient is formulated with a carrier such as sugar and acacia, tragacanth or gelatin and glycerin.

Compositions for rectal administration include suppositories containing a conventional suppository base such as cocoa butter.

Compositions suitable for transdermal administration include ointments, gels and patches.

Other compositions known to those skilled in the art can also be applied for percutaneous or subcutaneous administration, such as plasters.

Further, in preparing such pharmaceutical compositions comprising the active ingredient or ingredients in admixture with components necessary for the formulation of the compositions, other conventional pharmacologically acceptable additives may be incorporated, for example, excipients, stabilizers, antiseptics, wetting agents, emulsifying agents, lubricants, sweetening agents, coloring agents, flavoring agents, isotonicity agents, buffering agents, antioxidants and the like. As the additives, there may be mentioned, for example, starch, sucrose, fructose, dextrose, lactose, glucose, mannitol, sorbitol, precipitated calcium carbonate, crystalline cellulose, carboxymethylcellulose, dextrin, gelatin, acacia, EDTA, magnesium stearate, talc, hydroxypropylmethylcellulose, sodium metabisulfite, and the like.

In certain embodiments, the agents employed in the methods of the present invention are capable of crossing the blood-brain barrier (BBB), and/or are administered to facilitate delivery to the CNS. Oral, sublingual, parenteral, implantation, nasal and inhalational routes can provide delivery of the active agent to the CNS. Compounds of the present invention can be used in conjunction with delivery systems that facilitate delivery of the agents to the central nervous system. For example, various blood brain barrier permeability enhancers can be used, if desired, to transiently and reversibly increase the permeability of the blood brain barrier to a treatment agent. Such BBB permeability enhancers may include leukotrienes, bradykinin agonists, histamine, tight junction disruptors (e.g., zonulin, zot), hyperosmotic solutions (e.g., mannitol), cytoskeletal contracting agents, short chain alkylglycerols (e.g., 1-O-pentylglycerol), and others which are currently known in the art. In some embodiments, the compounds of the present invention can be administered to the CNS with minimal effects on the peripheral nervous system.

In further embodiments, the present invention provides kits including one or more containers comprising pharmaceutical dosage units comprising an effective amount of one or more compounds of the present invention.

4. Prodrugs

The present invention further provides prodrugs comprising the compounds described herein. The prodrugs can be formed utilizing a hydrolyzable coupling to the compounds described herein. Further discussions of prodrugs can be found in “Lessons Learned from Marketed and Investigational Prodrugs”, Ettnayer. P., Amidon, G. L., Clement. B. and Testa, B., J. Med. Chem., 2004, 47 (10), 2394-2404 and the monograph “Hydrolysis in Drug and Prodrug Metabolism: Chemistry, Biochemistry and Enzymology”, Testa. B. and Mayer, J. M., Wiley-Verlag Helvetica Chimica Acta, Zuerich, 2003, Chapters 1-12, pp. 1-780.

The term “prodrug” is intended to refer to a compound that is converted under physiological conditions, by solvolysis or metabolically to a specified compound that is pharmaceutically/pharmacologically active. The “prodrug” can be a compound of the present invention that has been chemically derivatized such that, (i) it retains some, all or none of the bioactivity of its parent drug compound, and (ii) it is metabolized in a subject to yield the parent drug compound. The prodrug of the present invention may also be a “partial prodrug” in that the compound has been chemically derivatized such that, (i) it retains some, all or none of the bioactivity of its parent drug compound, and (ii) it is metabolized in a subject to yield a biologically active derivative of the compound.

Prodrugs of the present invention are capable of passage across the blood-brain barrier and may undergo hydrolysis by CNS esterases to provide the active compound. Further, the prodrugs provided herein may also exhibit improved bioavailability, improved aqueous solubility, improved passive intestinal absorption, improved transporter-mediated intestinal absorption, protection against accelerated metabolism, tissue-selective delivery and/or passive enrichment in the target tissue.

Prodrugs of the present invention can include compounds described herein. For example, prodrugs of the present invention can include bumetanide, bumetanide aldehyde, bumetanide methyl ester, bumetanide cyanomethyl ester, bumetanide ethyl ester, bumetanide isoamyl ester, bumetanide octyl ester, bumetanide benzyl ester, bumetanide dibenzylamide, bumetanide diethylamide, bumetanide morpholinoethyl ester, bumetanide 3-(dimethylaminopropyl) ester, bumetanide N,N-diethylglycolamide ester, bumetanide dimethylolycolamide ester, bumetanide pivaxetil ester, furosemide, furosemide ethyl ester, furosemide cyanomethyl ester, furosernide benzyl ester, furosemide morpholinoethyl ester, furosemide 3-(dimethylaminopropyl) ester, furosemide N,N-diethylglycolamide ester, furosemide dibenzylamide, furosemide benzyltrimethylammonium salt, furosemide cetyltrimethylammonium salt, furosemide N,N-dimethylglycolamide ester, furosemide pivaxetil ester, furosemide propaxetil ester, piretanide, piretanide methyl ester, piretanide cyanomethyl ester, piretanide benzyl ester, piretanide morpholinoethyl ester, piretanide 3-(dimethylaminopropyl) ester, piretanide N,N-diethylglycolamide ester, piretanide diethylamide, piretanide dibenzylamide, piretanide benzylltrimethylammonium salt, piretanide cetylltrimethylammonium salt, piretanide N,N-dimethylglycolamide ester, piretanide pivaxetil ester, piretanide propaxetil ester, tetrazolyl-substituted azosemides, pyridinium-substituted torsemide salts (also termed pyridine-substituted torsemide quaternary ammonium salts), as well as similar acid, acid salt, ester and amido derivatives of S-thiobumetanide, O-thiobumetanide, dithiobumetanide, S-thiofurosemide, O-thiourosemide, dithiourosemide, S-hiopiretanide, O-thiopiretanide and dithiopiretanide. See previously presented schemes.

Moreover, as shown in the previously presented schemes, prodrugs can be formed by attachment of biocompatible polymers ethylene, such as those previously described including polyethylene glycol (PEG), to compounds of the present invention using link-ages degradable under physiological conditions. See also Schacht, E. H. et al. Poly(ethylene glycol) Chemistry and Biological Applications, American Chemical Society, San Francisco, Calif. 297-315 (1997). Attachment of PEG to proteins can be employed to reduce immunogenicity and/or extend the half-life of the compounds provided herein. Any conventional PEGylation method can be employed, provided that the PEGylated agent retains at least some pharmaceutical activity.

5. Methods of Use

The compounds of formula I, II, III, IV, V, VI, VII and/or VIII of the present invention as well as the prodrugs and modified diuretic or diuretic-like compounds described herein can be used for the regulation, including prevention, management and treatment, of a range of CNS conditions including, but not limited to, neuropathic pain, seizures, seizure disorders, epilepsy, status epilepticus, migraine headache, cortical spreading depression, headache, intracranial hypertension, central nervous system edema, neuropsychiatric disorders, neurotoxicity, head trauma, stroke, ischemia, hypoxia, anxiety, depression, Alzheimer's Disease, obesity, Parkinson's Disease, smoking cessation, additive disorders such as alcohol addiction, addiction to narcotics (such as cocaine addiction, heroin addiction, opiate addiction, etc.), anxiety and neuroprotection (e.g. reducing damage following stroke, reducing damage from neurodegenerative diseases like Alzheimer's, protecting against toxicity damage from ethanol. Accordingly, compounds of the present invention as well as the prodrugs and modified diuretic or diuretic-like compounds described herein can be used for the regulation of psychiatric disorders and neurological disorders and to modulate neuronal synchronization as well as improve CNS function.

By the terms “treating or “treatment” of a CNS disorder, it is intended that the severity of the disorder or the symptoms of the disorder are reduced, or the disorder is partially or entirely eliminated, as compared to that which would occur in the absence of treatment. Treatment does not require the achievement of a complete cure of the disorder. By the terms “preventing” or “prevention” of the CNS disorder, it is intended that the inventive methods eliminate or reduce the incidence or onset of the disorder, as compared to that which would occur in the absence of treatment. Alternatively stated, the present methods slow, delay, control, or decrease the likelihood or probability of the disorder in the subject, as compared to that which would occur in the absence of treatment.

Subjects suitable to be treated according to the present invention include, but are not limited to, avian and mammalian subjects, and are preferably mammalian. Mammals of the present invention include, but are not limited to, canines, felines, bovines, caprines, equines, ovines, porcines, rodents (e.g. rats and mice), lagomorphs, primates, humans, and the like, and mammals in utero. Any mammalian subject in need of being treated according to the present invention is suitable. Human subjects are preferred. Human subjects of both genders and at any stage of development (i.e., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and birds in ovo.

The present invention is primarily concerned with the treatment of human subjects, but the invention can also be carried out on animal subjects, particularly mammalian subjects such as mice, rats, dogs, cats, livestock and horses for veterinary purposes, and for drug screening and drug development purposes.

In therapeutic use for treatment of conditions in mammals (i.e. humans or animals) for which the compounds of the present invention or an appropriate pharmaceutical composition thereof are effective, the compounds of the present invention may be administered in an effective amount.

Since the activity of the compounds and the degree of the therapeutic effect vary, the actual dosage administered will be determined based upon generally recognized factors such as age, condition of the subject, route of delivery and body weight of the subject. The dosage can be from about 0.1 to about 100 mg/kg, administered orally 1 to 4 times per day. In addition, compounds can be administered by injection at approximately 0.01 to 20 mg/kg per dose, with administration 1 to 4 times per day. Treatment could continue for weeks, months or longer, as appropriate. Determination of optimal dosages for a particular situation is within the capabilities of those skilled in the art. See e.g., Remington, The Science And Practice of Pharmacy. 20th Edition, (Gennaro, A. R., Chief Editor), Philadelphia College of Pharmacy and Science, 2000.

In some embodiments, a therapeutically effective daily dose may be from about 0.001 mg to about 20 mg/kg of body weight per day of a compound of the present invention (0.07 mg/day to 1.40 grams/day for a 70 kg adult), or a pharmaceutically acceptable salt thereof; in some embodiments, from about 0.01 mg to about 10 mg/kg of body weight per day (0.7 mg/day to 700 mg/day for a 70 kg adult), and in other embodiments, from about 0.1 mg to about 1 mg/kg of body weight per day (7 mg/day to 70 mg/day for a 70 kg adult).

In further embodiments, bumetanide analogs according to the present invention may be administered 1.5 to 6 mg daily, for example, 1 tablet or capsule three times a day. In some embodiments, furosemide analogs according to the present invention may be administered 60 to 240 mg/day, for example, 1 tablet or capsule three times a day. In other embodiments, piretanide analogs according to the present invention may be administered 10 to 20 mg daily, for example. 1 tablet or capsule once a day. In some embodiments, azosemide analogs according to the present invention may be administered 60 mg per day. In other embodiments, torsemide analogs according to the present invention may be administered 10 to 20 mg daily, for example, 1 tablet or capsule once a day. It should be noted that lower doses may be administered, particularly for IV administration. Moreover, administration of a lower dose than administered for the parent compound may prevent undesirable peripheral effects such as diuresis.

In some embodiments, compounds of the present invention may have increased lipophilicity and/or reduced diuretic effects compared to the diuretic or diuretic-like compounds from which they are derived. In further embodiments, the compounds of the present invention may result in fewer undesirable side effects when employed in the regulatory, i.e., preventive, management and/or treatment, methods described herein.

In some embodiments, the level of diuresis that occurs following administration of an effective amount of a compound provided below as Formula I-VIII, is less than about 99%, 90%, 80%, 70%, 60%, 50%, 40%, 30%, 20% or 10% of that which occurs following administration of an effective amount of the parent molecule from which the compound is derived. For example, the compound may be less diuretic than the parent molecule when administered at the same mg/kg dose. Alternatively, the compound may be more potent than the parent molecule from which it is derived, so that a smaller dose of the compound may be required for effective relief of symptoms, and thus, may elicit less of a diuretic effect. Similarly, the compound may have a longer duration of effect in treating disorders than the parent molecule. Accordingly, compounds of the present invention may be administered less frequently than the parent molecule, and thus may lead to a lower total diuretic effect within any given period of time.

Further, it will be understood that the compositions of the present invention may be formulated to provide immediate release of the active ingredient or sustained or controlled release of the active ingredient. In a sustained release or controlled release preparation, release of the active ingredient may occur at a rate such that blood levels are maintained within an therapeutic range but below toxic levels over an extended period of time, e.g., 4 to 24 hours or even longer.

According to embodiments of the present invention, the amount of compound present in a prodrug or pharmaceutical preparation of the present invention includes an amount effective for regulating a range of CNS conditions including, but not limited to, neuropathic pain, seizures, seizure disorders, epilepsy, status epilepticus, migraine headache, cortical spreading depression, headache, intracranial hypertension, central nervous system edema, neuropsychiatric disorders, neurotoxicity, head trauma, stroke, ischemia, hypoxia, anxiety, depression, Alzheimer's Disease, obesity, Parkinson's Disease, smoking cessation, additive disorders such as alcohol addiction, addition to narcotics (such as cocaine addiction, heroin addiction, opiate addiction, etc.), anxiety and neuroprotection (e.g. reducing damage following stroke, reducing damage from neurodegenerative diseases like Alzheimer's, protecting against toxicity damage from ethanol), psychiatric disorders, neurological disorders, neuronal synchronization and general CNS function.

According to further embodiments of the present invention, a pharmaceutical preparation of the present invention may be administered alone or, optionally, in combination with a second agent. Suitable second agents include those useful for the prevention and/or treatment of a range of CNS conditions including, but not limited to, neuropathic pain, seizures, seizure disorders, epilepsy, status epilepticus, migraine headache, cortical spreading depression, headache, intracranial hypertension, central nervous system edema, neuropsychiatric disorders, neurotoxicity, head trauma, stroke, ischemia, hypoxia, anxiety, depression, Alzheimer's Disease, obesity, Parkinson's Disease, smoking cessation, additive disorders such as alcohol addiction, addiction to narcotics (such as cocaine addiction, heroin addiction, opiate addiction, etc.), anxiety and neuroprotection (e.g. reducing damage following stroke, reducing damage from neurodegenerative diseases like Alzheimer's, protecting against toxicity damage from ethanol). Accordingly, second agents for treatment in combination with compositions of the present invention include, but are not limited to, phenytoin, carbamazepine, barbiturates, phenobarbital, phenobarbital, mephobarbital, trimethadione, mephenytoin, paramethadione, phenthenylate, phenacemide, metharbital, benzchlorpropamide, phensuximide, primidone, methsuximide, ethotoin, aminoglutethinide, diazepam, clonazepam, clorazepate, fosphenytoin, ethosuximide, valproate, felbamate, gabapentin, lamotrigine, topiramate, vigrabatrin, tiagabine, zonisamide, clobazam, thiopental, midazolam, propofol, levetiracetam. oxcarbazepine, CCPene, GYK152466, serotonin receptor agonists, ergotamine, dihydroergotamine, sumatriptan, propranolol, metoprolol, atenolol, timolol, nadolol, nifeddipine, nimodipine, verapamil, aspirin, ketoprofen, tofenamic acid, mefenamic acid, naproxen, methysergide, paracetamol, clonidine, lisuride, iprazochrome, butalbital, benzodiazepines, divalproex sodium and other similar classes of compounds. See U.S. Pat. No. 6,495,601 to Hochman and U.S. Patent Application Serial No. 2002/0082252 to Hochman.

Further embodiments of the present invention will now be described with reference to the following examples. It should be appreciated that these examples are for the purposes of illustrating embodiments of the present invention, and do not limit the scope of the invention.

### EXAMPLE 1

### Methyl 3-Aminosulfonyl-5-butylamino-4-phenoxybenzoate (Bumetanide Methyl Ester)

To a slurry of bumetanide (1.2 g, 3.29 mmol) in methanol (12 mL) under nitrogen, was added a mixture of thionyl chloride (70 uL) in methanol (6 mL) over 5 minutes. After stirring for 5 minutes the reaction mixture became soluble. The reaction stirred for an additional 30 minutes, at which time the reaction was complete by thin layer chromatography (TLC). The methanol was removed under reduced pressure and the residue was brought up in ethyl acetate and washed with saturated sodium bicarbonate, water and brine. The ethyl acetate was dried over anhydrous magnesium sulfate and concentrated to a yield 1.1 g (89%) of methyl 3-aminosulfonyl-5-butylamino-4-phenoxybenzoate as a white solid. Using similar methodology bumetanide ethyl ester, bumetanide isoamyl ester, bumetanide octyl ester and bumetanide benzyl ester, can be prepared.

### EXAMPLE 2

### 3-Aminosulfonyl-5-butylamino-4-phenoxythiobenzoic Acid (Thiobumetanide, Bumetanide —(C═O)—SH Thioacid)

Bumetanide can be reacted thionyl chloride to make the corresponding acid chloride which can then be reacted with sodium hydrogen sulfide to, give 3-aminosulfonyl-5-butylamino-4-phenoxythiobenzoic acid (thiobumetanide, S-bumetanide thioacid) by the methodology of Noble, P. and Tarbell, D. S., Org. Synth., Coll. Vol. IV, John Wiley & Sons, Inc., New York. 1963, 924-927.

### EXAMPLE 3

### 3-Aminosulfonyl-5-butylamino-4-phenoxythiobenzoic Acid (Thiobumetanide, Bumetanide —(C═O)—SH Thioacid)

Bumetanide methyl ester can be reacted with hydrogen sulfide or sodium hydrogen sulfide to give, following acidification, 3-aminosulfonyl-5-butylamino-4-phenoxythiobenzoic acid (thiobumetanide, bumetanide thioacid).

### EXAMPLE 4

### Thiomethyl 3-Aminosulfonyl-5-butylamino-4-phenoxybenzoate (Bumetanide S-Methyl Thioster)

In like manner to Example 1, bumetanide can be reacted with a catalytic amount of thionyl chloride in methanethiol (methyl mercaptan) to give thiomethyl 3-aminosulfonyl-5-butylamino-4-phenoxybenzoate. Using similar methodology with bumetanide and the corresponding thiols, bumetanide S-ethyl thioester, bumetanide S-isoamyl thioester, bumetanide S-octyl thioester and bumetanide S-benzyl thioester, can be prepared. Using similar methodology with dithiobumetanide and the corresponding alcohols, bumetanide O-ethyl thioester, bumetanide O-isoamyl thioester, bumetanide O-octyl thioester and bumetanide O-benzyl thioester, can be prepared.

### EXAMPLE 5

### 3-Aminosulfonyl-5-butylamino-4-phenoxy-dithiobenzoic Acid (Dithiobumetanide, Bumetanide —(C═S)—SH Dithioacid)

Thiobumetanide can be reacted thionyl chloride to make the corresponding thioacid chloride which can then be reacted with sodium hydrogen sulfide to give 3-aminosulfonyl-5-butylamino-4-phenoxy-dithiobenzoic acid (dithiobumetanide, bumetanide dithioacid) by the methodology of Noble, P. and Tarbell, D. S., Org. Synth., Coll. Vol. IV, John Wiley & Sons, Inc., New York, 1963, 924-927.

### EXAMPLE 6

### Methyl 3-Aminosulfonyl-5-butylamino-4-phenoxy-dithiobenzoate (Bumetanide Methyl Dithioester)

In like manner to Example 1, dithiobumetanide can be reacted with a catalytic amount of thionyl chloride in methanethiol (methyl mercaptan) to give methyl 3-aminosulfonyl-5-butylamino-4-phenoxydithiobenzoate, Using similar methodology bumetanide ethyl dithioester, bumetanide isoamyl dithioester, bumetanide octyl dithioester and bumetanide benzyl dithioester, can be prepared.

### EXAMPLE 7

### Cyanomethyl 3-Aminosulfonyl-5-butylamino-4-phenoxybenzoate (Bumetanide Cyanomethyl Ester)

Bumetanide (1.0 g, 2.7 mmol) was dissolved in dimethylformamide (DMF) and chloroacetonitrile (195 uL, 2.7 mmol) was added followed by triethylamine (465 uL). The reaction was heated to 100° C. for 12 hours, TLC and liquid chromatography-coupled mass spectrometry (LC/MS) indicated the reaction was complete. The reaction was cooled to room temperature brought up in dichloromethane and washed with water, saturated ammonium chloride and reduced to a slurry, To the slurry was added water (25 mL) and crude product precipitated as an off white solid. Pure cyanomethyl 3-3minosulfonyl-5-butylamino-4-phenoxybenzoate (850 mg) was obtained via recrystallization in acetonitrile. Using similar methodology bumetanide ethyl ester, bumetanide isoamyl ester, bumetanide octyl ester, and bumetanide benzyl ester, can be prepared.

### EXAMPLE 8

### Benzyl 3-Aminosulfonyl-5-butylamino-4-phenoxybenzoate (Bumetanide Benzyl Ester)

Bumetanide (1.15 g, 3.15 mmol) was dissolved in dimethylformamide (DMF, 10 mL) and benzyl chloride (400 ul, 2.8 mmol) was added followed by triethylamine (480 uL). The reaction was heated to 80° C. for 12 hours, TLC and LC/MS indicated the reaction was complete. The reaction was cooled to room temperature brought up in dichloromethane and washed with water, saturated ammonium chloride and concentrated to a thick slurry. To the slurry was added water (25 mL), the resultant solids were filtered and dried in a vacuum oven at 50° C. for 12 hours to yield 1.0 g (80%) of benzyl 3-aminosulfonyl-5-butylamino-4-phenoxybenzoate.

### EXAMPLE 9

### 2-(4-Morpholino)ethyl 3-Aminosulfonyl-5-butylamino-4-phenoxybenzoate (Bumetanide Morpholinoethyl Ester)

Bumetanide (1.2 g, 3.299 mmol) was dissolved in dimethylformamide (DMF, 12 mL) and 4-(2-chloroethyl)morpholine hydrochloride (675 mg, 3.62 mmol) was added followed by triethylamine (1 mL) and sodium iodide (500 mg 3.33 mmol). The reaction was heated to 95° C. for 8 hours, TLC and LC/MS indicated the reaction was complete. The reaction was cooled to room temperature brought up in dichloromethane and washed with water, saturated ammonium chloride and concentrated to dryness. After purification via biotage flash chromatography, the purified elute, on evaporation under vacuum, yielded 2-(4-morpholino)ethyl 3-aminosulfonyl-5-butylamino-4-phenoxybenzoate as a white solid (600 mg, 62%).

### EXAMPLE 10

### 3-(N,N-Dimethylaminopropyl 3-Aminosulfonyl-5-butylamino-4-phenoxybenzoate [Bumetanide 3-(Dimethylaminopropyl) Ester]

In similar manner to Example 54, bumetanide can be reacted with 3-(dimethylamino)propyl chloride hydrochloride, triethylamine and sodium iodide in dimethylformamide (DMF) to yield 3-(N,N-dimethylaminopropyl 3-aminosulfonyl-5-butylamino-4-phenoxybenzoate.

### EXAMPLE 11

### 3-(N,N-Dimethylaminopropyl 3-Aminosulfonyl-5-butylamino-4-phenoxy-dithiobenzoate [Bumetanide 3-(Dimethylaminopropyl) Dithioester]

In similar manner to Example 10, dithiobumetanide can be reacted with 3-(dimethylamino)propyl chloride hydrochloride, triethylamine and sodium iodide in dimethylformamide (DMF) to yield 3-(N,N-dimethylaminopropyl 3-aminosulfonyl-5-butylamino-4-phenoxy-dithiobenzoate.

### EXAMPLE 12

### N,N-Diethylaminocarbonylmethyl 3-Aminosulfonyl-5-butylamino-4-phenoxybenzoate (Bumetanide N,N-Diethylglycolamido Ester)

Bumetanide (1.2 g, 3.29 mmol) was dissolved in dimethylformamide (12 mL) and 2-chloro-N,N-diethylacetamide (500 mg, 3.35 mmol) was added followed by triethylamine (0.68 mL) and sodium iodide (500 mg 3.33 mmol). The reaction was heated to 95° C. for 8 hours, TLC and LC/MS indicated the reaction was complete. The reaction was cooled to room temperature brought up in dichloromethane and washed with water, saturated ammonium chloride and reduced to a thick slurry. To the slurry was added water (25 mL), the resultant solids precipitated from the solution. The product was filtered and dried in a vacuum oven at 50° C. for 12 hours to yield 1.0 g of N,N-diethylaminocarbonylmethyl 3-aminosulfonyl-5-butylamino-4-phenoxybenzoate.

### EXAMPLE 13

### N,N-Diethyl 3-Aminosulfonyl-5-butylamino-4-phenoxybenzamide (Bumetanide Diethylamide)

Bumetanide (1.16 g, 3.2 mmol) was dissolved in dichloromethane (10 mL) and 1-ethyl-3-(3-dimethylaminopropyl)carbodiimide (EDC, 690 mg, 3.6 mmol) was added and after 5 minutes N-hydroxybenzotriazole (HOBt , 498 mg, 3.6 mmol) was added and the solution was allowed to stir for an additional 5 minutes. Diethylamine (332 uL, 3.2 mmol) was added and the reaction was stirred for 2 hours. The reaction was washed with washed with saturated sodium bicarbonate, water, brine and dried with magnesium sulfate. The dichloromethane was removed under reduced pressure to yield 860 mg (65%) of pure N,N-diethyl 3-aminosulfonyl-5-butylamino-4-phenoxybenzamide.

### EXAMPLE 14

### N,N-Diethyl 3-Aminosulfonyl-5-butylamino-4-phenoxythiobenzamide (Bumetanide Diethylthioamide)

In similar manner to Example 5, dithiobumetanide can be reacted with thionyl chloride to give the thioacid chloride, which can be reacted with diethylamime to afford N,N-diethyl 3-aminosulfonyl-5-butylamino-4-phenoxythiobenzamide.

### EXAMPLE 15

### N,N-Dibenzyl 3-Aminosulfonyl-5-butylamino-4-phenoxybenzamide (Bumetanide Dibenzylamide)

Bumetanide (960 mg, 2.6 mmol) was dissolved in dimethylformamide (DMF, 10 mL) and 1-ethyl-3-(3-dimethylaminopropyl)carbodiimide (EDC, 560 mg, 3.6 mmol) was added and after 10 minutes 1-hydroxybenzotriazole (HOBt, 392 mg, 2.9 mmol) was added and the solution was allowed to stir for an additional 10 minutes. Dibenzylamine (1 mL, 5.2 mmol) was added and the reaction was stirred for 2 hours, at which time the reaction was complete by LC/MS. The reaction was poured into saturated ammonium chloride (20 mL) and extracted with ethyl acetate (2×100 mL). The ethyl acetate was washed with saturated sodium bicarbonate, water, brine and dried over anhydrous magnesium sulfate. The ethyl acetate was removed under reduced pressure to yield 1.0 g (75%) of N,N-dibenzyl 3-aminosulfonyl-5-butylamino-4-phenoxybenzamide as white solid.

### EXAMPLE 16

### Benzyltrimethylammonium 3-Aminosulfonyl-5-butylamino-4-phenoxybenzoate (Bumetanide Benzylltrimethylammonium Salt)

To a solution of benzyltrimethylammonium hydroxide (451 mg, 2.7 mmol) in water (10 mL) was added bumetanide (1 g, 2.7 mmol) over a period of 5 minutes. The reaction mixture became clear after 10 minutes of stirring. The water was removed under reduced pressure to yield a crude colorless oil. Pure product was obtained from recrystallization of the oil with water and heptane to yield 690 mg of benzyltrimethylammonium 3-aminosulfonyl-5-butylamino-4-phenoxybenzoate as light pink crystals.

### EXAMPLE 17

### Cetyltrimethylammonium 3-Aminosulfonyl-5-butylamino-4-phenoxybenzoate (Bumetanide Cetylltrimethylammonium Salt)

In similar manner to Example 16, bumetanide can be reacted with cetyltrimethylammonium hydroxide in water to yield cetyltrimethylammonium 3-aminosulfonyl-5-butylamino-4-phenoxybenzoate.

### EXAMPLE 18

### N,N-Dimethylaminocarbonylmethyl 3-Aminosulfonyl-5-butylamino-4-phenoxybenzoate (Bumetanide N,N-Dimethylglycolamido Ester)

Bumetanide (1.2 g, 3.29 mmol) was dissolved in dimethylformamide (DMF, 10 mL) and 2-chloro-N,N-dimethylacetamide (410 uL, 3.9 mmol) was added followed by triethylamine (0.70 mL) and sodium iodide (545 mg, 3.6 mmol). The reaction was heated to 50° C. for 10 hours, TLC and LC/MS indicated the reaction was complete. The solvent was removed under reduced pressure and the residue was dissolved in ethyl acetate and washed with saturated sodium bicarbonate, water, and brine and dried over anhydrous magnesium sulfate. The ethyl acetate was removed under reduced pressure and the product was purified via flash chromatography to yield 685 mg (60%) of pure NN-dimethylaminocarbonylmethyl 3-aminosulfonyl-5-butylamino-4-phenoxybenzoate.

### EXAMPLE 19

### t-Buhtylcarbonyloxymethyl 3-Aminosulfonyl-5-butylamino-4-phenoxybenzoate (Bumetanide Pivaxetil Ester)

Bumetanide (1.2 g, 32.9 mmol) was dissolved in dimethylformamide (DMF, 10 mL) and chloromethyl pivalate (575 uL, 3.9 mmol) was added followed by triethylamine (0.70 mL) and sodium iodide (545 mg, 3.6 mmol). The reaction was heated to 50° C. for 10 hours, TLC and LC/MS indicated the reaction was complete. The solvent was removed under reduced pressure and the residue was dissolved in ethyl acetate and washed with saturated sodium bicarbonate, water, and brine and dried over anhydrous magnesium sulfate. The ethyl acetate was removed under reduced pressure and the product was purified via flash chromatography to yield 653 mg (60%) of pure t-butylcarbonyloxymethyl 3-aminosulfonyl-5-butylamino-4-phenoxybenzoate.

### EXAMPLE 20

### t-Butylcarbonyloxymethyl 3-Aminosulfonyl-5-butylamino-4-phenoxy-dithiobenzoate (Bumetanide Pivaxetil Dithioester)

In similar manner to Example 19, dithiobumetanide can be reacted with chloromethyl pivalate, triethylamine and sodium iodide in dimethylformamide (DMF) to yield t-butylcarbonyloxymethyl 3-aminosulfonyl-5-butylamino-4-phenoxy-dithiobenzoate.

### EXAMPLE 21

### Ethylcarbonyloxymethyl 3-Aminosulfonyl-5-butylamino-4-phenoxybenzoate (Bumetanide Propaxetil Ester)

In similar manner to Example 19, bumetanide can be reacted with chloromethyl propionate, triethylamine and sodium iodide in dimethylformamide (DMF) to yield ethylcarbonyloxymethyl 3-aminosulfonyl-5-butylamino-4-phenoxybenzoate.

### EXAMPLE 22

### Ethylcarbonyloxymethyl 3-Aminosulfonyl-5-Butylamino-4-phenoxy-dithiobenzoate (Bumetanide Propaxetil Dithioester)

In similar manner to Example 21, dithiobumetanide can be reacted with chloromethyl propionate, triethylamine and sodium iodide in dimethylformamide (DMF) to yield ethylcarbonyloxymethyl 3-aminosulfonyl-5-butylamino-4-phenoxy-dithiobenzoate.

### EXAMPLE 23

### Methyl 3-Aminosulfonyl-4-phenoxy-5-(1-pyrrolidinyl)benzoate (Piretanide Methyl Ester)

In similar manner to Example 1, piretanide can be reacted with thionyl chloride and methanol to yield methyl 3-aminosulfonyl-4-phenoxy-5-(1-pyrrolidinyl)benzoate. Using similar methodology piretanide ethyl ester, piretanide isoamyl ester, piretanide octyl ester and piretanide benzyl ester can be prepared.

### EXAMPLE 24

### 3-Aminosulfonyl-4-phenoxy-5-(1-pyrrolidinyl)-thiobenzoic Acid (Thiopiretanide, Piretanide —(C═O)—SH Thioacid)

Piretanide can be reacted thionyl chloride to make the corresponding acid chloride which can then be reacted with sodium hydrogen sulfide to Give 3-aminosulfonyl-4-phenoxy-5-(1-pyrrolidinyl)-thiobenzoic acid (thiopiretanide, S-piretanide thioacid) by the methodology of Noble, P. and Tarbell, D. S., Org. Synth., Coll. Vol. IV, John Wiley & Sons, Inc., New York, 1963, 924-927.

### EXAMPLE 25

### 3-Aminosulfonyl-4-phenoxy-5-(1-pyrrolidinyl)-thiobenzoic Acid (Thiopiretanide, Piretanide —(C═O)—SH Thioacid)

Piretanide methyl ester can be reacted with hydrogen sulfide or sodium hydrogen sulfide to give 3-aminosulfonyl-4-phenoxy-5-(1-pyrrolidinyl)-thiobenzoic acid (thiopiretanide, S-piretanide thioacid).

### EXAMPLE 26

### Thiomethyl 3-aminosulfonyl-4-phenoxy-5-(1-pyrrolidinyl)benzoate (Piretanide S-Methyl Thioester)

In like manner to Example 1, piretanide can be reacted with a catalytic amount of thionyl chloride in methanethiol (methyl mercaptan) to give thiomethyl 3-aminosulfonyl-4-phenoxy-5-(1-pyrrolidinyl)benzoate. Using similar methodology with piretanide and the corresponding thiols, piretanide S-ethyl thioester, piretanide S-isoamyl thioester, piretanide S-octyl thioester and piretanide S-benzyl thioester, can be prepared. Using similar methodology with dithiopiretanide and the corresponding alcohols, piretanide O-ethyl thioester, piretanide O-isoamyl thioester, piretanide O-octyl thioester and piretanide O-benzyl thioester, can be prepared.

### EXAMPLE 27

### 3-Aminosulfonyl-4-phenoxy-5-(1-pyrrolidinyl)-dithiobenzoic Acid (Dithiopiretanide, Piretanide —(C═S)—SH Dithioacid)

Thiopiretanide can be reacted thionyl chloride to make the corresponding thioacid chloride which can then be reacted with sodium hydrogen sulfide to give 3-aminosulfonyl-4-phenoxy-5-(1-pyrrolidinyl)-dithiobenzoic acid (dithiopiretanide, piretanide dithioacid) by the methodology of Noble, P. and Tarbell, D. S., Org. Synth., Coll. Vol. IV, John Wiley & Sons, Inc., New York, 1963, 924-927.

### EXAMPLE 28

### Methyl 3-aminosulfonyl-4-phenoxy-5-(1-pyrrolidinyl)-dithiobenzoate (Piretanide Methyl Dithioester)

In like manner to Example 1, dithiopiretanide can be reacted with a catalytic amount of thionyl chloride in methanethiol (methyl mercaptan) to give methyl 3-aminosulfonyl-4-phenoxy-5-(1-pyrrolidinyl)-dithiobenzoate. Using similar methodology piretanide ethyl dithioester, piretanide isoamyl dithioester, piretanide octyl dithioester and piretanide benzyl dithioester can be prepared.

### EXAMPLE 29

### Cyanomethyl 3-Aminosulfonyl-4-phenoxy-5-(1-Pyrrolidinyl)benzoate (Piretanide Cyanomethyl Ester)

In similar manner to Example 7, piretanide can be reacted with chloroacetonitrile and triethylamine in DMF to yield cyanomethyl 3-aminosulfonyl-4-phenoxy-5-(1-pyrrolidinyl)benzoate.

### EXAMPLE 30

### Benzyl 3-Aminosulfonyl-4-phenoxy-5-(1-pyrrolidinyl)benzoate (Piretanide Benzyl Ester)

In similar manner to Example 8, piretanide can be reacted with benzyl chloride and triethylamine in DMF to yield benzyl 3-aminosulfonyl-4-phenoxy-5-(1-pyrrolidinyl)benzoate.

### EXAMPLE 31

### 2-(4-Morpholino)ethyl 3-Aminosulfonyl-4-phenoxy-5-(1-pyrrolidinyl)benzoate (Piretanide Morpholinoethyl Ester)

In similar manner to Example 9, piretanide can be reacted with 4-(2-chloroethyl)morpholine hydrochloride, triethylamine and sodium iodide in DMF to yield 2-(4-morpholino)ethyl 4-aminosulfonyl-4-phenoxy-5-(1-pyrrolidinyl)benzoate.

### EXAMPLE 32

### 3-N,N-Dimethylaminopropyl 3-Aminosulfonyl-4-phenoxy-5-(1-pyrrolidinyl)benzoate [Piretanide 3-(Dimethylaminopropyl) Ester]

In similar manner to Example 54, piretanide can be reacted with 3-(dimethylamino)propyl chloride hydrochloride, triethylamine and sodium iodide in dimethylformamide (DMF) to yield 3-(N,N-dimethylaminopropyl 3-aminosulfonyl-4-phenoxy-5-(1-pyrrolidinyl)benzoate.

### EXAMPLE 33

### 3-(N,N-Dimethylaminopropyl 3-Aminosulfonyl-4-phenoxy-5-(1-pyrrolidinyl)dithiobenzoate [Piretanide 3-(Dimethylaminopropyl) Dithioester]

In similar manner to Example 32, dithiopiretanide can be reacted with 3-(dimethylamino)propyl chloride hydrochloride, triethylamine and sodium iodide in dimethylformamide (DMF) to yield 3-(N,N-dimethylaminopropyl 3-aminosulfonyl-4-phenoxy-5-(1-pyrrolidinyl)dithiobenzoate.

### EXAMPLE 34

### N,N-Diethylaminocarbonylmethyl 3-Aminosulfonyl-4-phenoxy-5-(1-pyrrolidinyl)benzoate (Piretanide N,N-Diethylglycolamide Ester)

In similar manner to Example 12, piretanide can be reacted with 2-chloro-N,N-diethylacetamide, triethylamine and sodium iodide in dimethylformamide (DMF) to yield N,N-diethylaminocarbonylmethyl 3-aminosulfonyl-4-phenoxy-5-(1-pyrrolidinyl)benzoate.

### EXAMPLE 35

### N,N-Diethyl 3-Aminosulfonyl-4-phenoxy-5-(1-pyrrolidinyl)benzoate (Piretanide Diethylamide)

In similar manner to Example 13, piretanide can be reacted with EDC, HOBt and diethylamine in DMF to yield N,N-diethyl 3-aminosulfonyl-4-phenoxy-5-(1-pyrrolidinyl)benzamide.

### EXAMPLE 36

### N,N-Diethyl 3-Aminosulfonyl-4-phenoxy-5-(1-pyrrolidinyl)benzoate (Piretanide Diethylthioamide)

In similar manner to Example 35, dithiopiretanide can be reacted with EDC, HOBt and diethylamine in DMF to yield N,N-diethyl 3-aminosulfonyl-4-phenoxy-5-(1-pyrrolidinyl)thiobenzamide.

### EXAMPLE 37

### N,N-Dibenzyl 3-Aminosulfonyl-4-phenoxy-5-(1-pyrrolidinyl)benzoate (Piretanide Dibenzylamide)

In similar manner to Example 15, piretanide can be reacted with EDC, HOBt and dibenzylamine in DMF to yield N,N-dibenzyl 3-aminosulfonyl-4-phenoxy-5-(1-pyrrolidinyl)benzamide.

### EXAMPLE 38

### Benzyltrimethylammonium 3-Aminosulfonyl-4-phenoxy-5-(1-pyrrolidinyl)benzoate (Piretanide Benzylltrimethylammonium Salt)

In similar manner to Example 16, piretanide can be reacted with Benzyltrimethylammonium hydroxide to yield benzyltrimethylammonium 3-aminosulfonyl-4-phenoxy-5-(1-pyrrolidinyl)benzoate.

### EXAMPLE 39

### Cetyltrimethylammonium 3-Aminosulfonyl-4-phenoxy-5-(1-pyrrolidinyl)benzoate (Piretanide Cetylltrimethylammonium Salt)

In similar manner to Example 17, piretanide can be reacted with cetyltrimethylammonium hydroxide in water to yield cetyltrimethylammonium 3-aminosulfonyl-4-phenoxy-5-(1-pyrrolidinyl)benzoate.

### EXAMPLE 40

### N,N-Dimethylaminocarbonylmethyl 3-Aminosulfonyl-4-phenoxy-5-(1-pyrrolidinyl)benzoate (Piretanide N,N-Dimethylglycolamido Ester)

In similar manner to Example 18, piretanide can be reacted with 2-chloro-N,N dimethylacetamide, triethylamine and sodium iodide in DMF to yield N,N-dimethylaminocarbonylmethyl 3-aminosulfonyl-4-phenoxy-5-(1-pyrrolidinyl)benzoate

### EXAMPLE 41

### t-Butylcarbonyloxymethyl 3-Aminosulfonyl-4-phenoxy-5-(1-pyrrolidinyl)benzoate (Piretanide Pivaxetil Ester)

In similar manner to Example 19, piretanide can be reacted with chloromethyl pivalate, triethylamine and sodium iodide in DMF to yield t-butylcarbonyloxymethyl 3-aminosulfonyl-4-phenoxy-5-(1-pyrrolidinyl)benzoate.

### EXAMPLE 42

### t-Butylcarbonyloxymethyl 3-Aminosulfonyl-4-phenoxy-5-(1-pyrrolidinyl)dithiobenzoate (Piretanide Pivaxetil Dithioester)

In similar manner to Example 41, dithiopiretanide can be reacted with chloromethyl pivalate, triethylamine and sodium iodide in DMF to yield t-butylcarbonyloxymethyl 3-aminosulfonyl-4-phenoxy-5-(1-pyrrolidinyl)dithiobenzoate.

### EXAMPLE 43

### Ethylcarbonyloxymethyl 3-Aminosulfonyl-4-phenoxy-5-(1-pyrrolidinyl)benzoate (Piretanide Propaxetil Ester)

In similar manner to Example 21, piretanide can be reacted with chloromethyl propionate, triethylamine and sodium iodide in DMF to yield ethylcarbonyloxymethyl 3-aminosulfonyl-4-phenoxy-5-(1-pyrrolidinyl)benzoate.

### EXAMPLE 44

### Ethyl 5-Aminosulfonyl-4-chloro-2-[(2-furanylmethyl)amino]benzoate (Furosemide Ethyl Ester)

The method of Bundgaard, H., Norgaard, T. and Nielsen, N. M., Int. J. Pharmaceutics, 1988, 42, 217-224, can be employed to prepare ethyl 5-aminosulfonyl-4-chloro-2-[(2-furanylmethyl)amino]benzoate, m.p. 163-165°. Using similar methodology furosemide methyl ester, furosemide isoamyl ester, furosemide octyl ester and furosemide benzyl ester can be prepared.

### EXAMPLE 45

### Methyl 5-Aminosulfonyl-4-chloro-2-[(2-furanylmethyl)amino benzoate (Furosemide Methyl Ester)

The method of Bundgaard, H., Norgaard, T. and Nielsen, N. M., Int. J. Pharmaceutics, 1988, 42, 217-224, can be employed to prepare methyl 5-aminosulfonyl-4-chloro-2-[(2-furanylmethyl)amino]benzoate.

### EXAMPLE 46

### 5-Aminosulfonyl-4-chloro-2-[(2-furanylmethylamino]thiobenzoic Acid (Thiofurosemide, Furosemide —(C═O)—SH Thioacid)

Furosemide can be reacted thionyl chloride to make the corresponding acid chloride which can then be reacted with sodium hydrogen sulfide to give 5-aminosulfonyl-4-chloro-2-[(2-furanylmethyl)amino]thiobenzoic acid (thiofurosemide, S-furosemide thioacid) by the methodology of Noble, P. and Tarbell, D. S., Org, Synth., Coll. Vol. IV, John Wiley & Sons, Inc., New York, 1963, 924-927.

### EXAMPLE 47

### 5-Aminosulfonyl-4-chloro-2-[(2-furanylmethylamino]thiobenzoic Acid (Thiofurosemide, Furosemide —(C═O)—SH Thioacid)

Furosemide methyl ester can be reacted with hydrogen sulfide or sodium hydrogen sulfide to give, following acidification, 3-aminosulfonyl-5-butylamino-4-phenoxythiobenzoic acid (thiofurosemide, S-furosemide thioacid).

### EXAMPLE 48

### Thiomethyl 5-Aminosulfonyl-4-chloro-2-[(2-furanylmethyl)amino]benzoate (Furosemide S-Methyl Thioester)

In like manner to Example 1, bumetanide can be reacted with a catalytic amount of thionyl chloride in methanethiol (methyl mercaptan) to give thiomethyl 5-aminosulfonyl-4-chloro-2-[(2-furanylmethyl)amino]benzoate. Using similar methodology with furosemide and the corresponding thiols, furosemide S-ethyl thioester, furosemide S-isoamyl thioester, furosemide S-octyl thioester and furosemide S-benzyl thioester, can be prepared. Using similar methodology with dithio furosemide and the corresponding alcohols, furosemide O-ethyl thioester, furosemide O-isoamyl thioester, furosemide O-octyl thioester and furosemide O-benzyl thioester, can be prepared.

### EXAMPLE 49

### 5-Aminosulfonyl-4-chloro-2-[(2-furanylmethyl)amino]-dithiobenzoic Acid (Dithiofurosemide, Furosemide —(C═S)—SH Dithioacid)

Thiofurosemide can be reacted thionyl chloride to make the corresponding thioacid chloride which can then be reacted with sodium hydrogen sulfide to give 5-aminosulfonyl-4-chloro-2-[(2-furanylmethyl)amino]-dithiobenzoic aciid (dithiofurosemide, furosemide dithioacid) by the methodology of Noble, P. and Tarbell, D. S., Org. Synth., Coll. Vol. IV, John Wiley & Sons. Inc., New York, 1963, 924-927.

### EXAMPLE 50

### Methyl 5-Aminosulfonyl-4-chloro-2-[(2-furanylmethyl)amino]dithiobenzoate (Furosemide Methyl Dithioester)

In like manner to Example 1, dithiofurosemide can be reacted with a catalytic amount of thionyl chloride in methanethiol (methyl mercaptan) to give methyl 5-aminosulfonyl-4-chloro-2-[(2-furanylmethyl)amino]dithiobenzoate. Using similar methodology furosemide ethyl dithioester, furosemide Ssoamyl dithioester, furosemide octyl dithioester and furosemide benzyl dithioester can be prepared.

### EXAMPLE 51

### Cyanomethyl 5-Aminosulfonyl-4-chloro-2-[(2-furanylmethyl)amino]benzoate (Furosemide Cyanomethyl Ester)

In similar manner to Example 7, furosemide can be reacted with chloroacetonitrile and triethylamine in DMF to yield cyanomethyl 5-aminosulfonyl-4-chloro-2-[(2-furanylmethyl)amino]benzoate.

### EXAMPLE 52

### Benzyl 5-Aminosulfonyl-4-chloro-2-[(2-furanylmethyl)amino]benzoate (Furosemide Benzyl Ester)

In similar manner to Example 8, furosemide can be reacted with benzyl chloride and triethylamine in DMF to yield benzyl 5-aminosulfonyl-4-chloro-2-[(2-furanylmethyl)amino]benzoate.

### EXAMPLE 53

### 2-(4-Morpholino)ethyl 5 -Aminosulfonyl-4-chloro-2-[(2-furanylmethyl)amino]benzoate (Furosemide Morpholinoethyl Ester)

The method of Mork, N., Bundgaard, H., Shalmi, M. and Christensen. S., Int. J. Pharmaceutics, 1990, 60, 163-169, can be employed to prepare 2-(4-morpholino)ethyl 5-aminosulfonyl-4-chloro-2-[(2-furanylmethyl)amino]benzoate, m.p. 134-135°.

### EXAMPLE 54

### 3-(N,N-Dimethylaminopropyl 5-Aminosulfonyl-4-chloro-2-[(2-furanylmethyl)amino]benzoate [Furosemide 3-(Dimethylaminopropyl) Ester]

The method of Mork, N., Bundgaard. H., Shalmi, M. and Christensen, S., Int. J. Pharmaceutics, 1990, 60, 163-169, can be employed to prepare 3-(N,N-dimethylaminopropyl 5-aminosulfonyl-4-chloro-2-[(2-furanylmethyl)amino]benzoate, m.p. 212-213°.

### EXAMPLE 55

### 3-(N,N-Dimethylaminopropyl 5-Aminosulfonyl-4-chloro-2-[(2-furanylmethyl)amino]dithiobenzoate [Furosemide 3-(Dimethylaminopropyl) Dithioester]

In similar manner to Example 54, dithiofurosemide can be reacted with 3-(dimethylamino)propyl chloride hydrochloride, triethylamine and sodium iodide in dimethylformamide (DMF) to yield 3-(N,N-dimethylaminopropyl 5-aminosulfonyl-4-chloro-2-[(2-furanylmethyl)amino]dithiobenzoate.

### EXAMPLE 56

### N,N-Diethylaminocarbonylmethyl 5-Aminosulfonyl-4-chloro-2-[(2-furanylmethyl)amino]benzoate (Furosemide N,N-Diethylglycolamido Ester)

The method of Mork, N., Bundgaard, H., Shalmi, M. and Christensen, S., Int. J. Pharmaceutics, 1990, 60, 163-169, can be employed to prepare N,N-diethylaminocarbonylmethyl 5-aminosulfonyl-4-chloro-2-[(2-furanylmethyl)amino]benzoate, m.p. 135-136°.

### EXAMPLE 57

### N,N-Diethyl 5-Aminosulfonyl-4-chloro-2-[(2-furanylmethyl)amino]benzamide (Furosemide Diethylamide)

In similar manner to Example 13, furosemide can be reacted with EDC, HOBt and diethylamine in DMF to yield N,N-diethyl 5-aminosulfonyl-4-chloro-2-[(2-furanylmethyl)amino]benzamide.

### EXAMPLE 58

### N,N-Diethyl 5-Aminosulfonyl-4-chloro-2-[(2-furanylmethyl)amino]benzamide (Furosemide Diethylthioamide)

In similar manner to Example 57, dithiofurosemide can be reacted with EDC, HOBt and diethylamine in DMF to yield N,N-diethyl 5-aminosulfonyl-4-chloro-2-[(2-furanylmethyl)amino]thiobenzamide.

### EXAMPLE 59

### N,N-Dibenzyl 5-Aminosulfonyl-4-chloro-2-[(2-furanylmethyl)amino]benzamide (Furosemide Dibenzylamide)

In similar manner to Example 15, furosemide can be reacted with EDC, HOBt and dibenzylamine in DMF to yield N,N-dibenzyl 5-aminosulfonyl-4-chloro-2-[(2-furanylmethyl)amino]benzamide.

### EXAMPLE 60

### Benzyltrimethylammonium 5-Aminosulfonyl-4-chloro-2-[(2-furanylmethyl)amino]benzoate (Furosemide Benzyltrimethylammonium Salt)

In similar manner to Example 16, furosemide can be reacted with benzyltrimethylammonium hydroxide to yield benzyltrimethylammonium 5-aminosulfonyl-4-chloro-2-[(2-furanylmethyl)amino]benzoate.

### EXAMPLE 61

### Cetyltrimethylammonium 5-Aminosulfonyl-4-chloro-2-[(2-furanylmethyl)amino]benzoate (Furosemide Cetyltrimethylammonium Salt)

In similar manner to Example 17, furosemide can be reacted with cetyltrimethylammonium hydroxide in water to yield cetyltrimethylammonium 5-aminosulfonyl-4-chloro-2-[(2-furanylmethyl)amino]benzoate.

### EXAMPLE 62

### N,N-Dimethylaminocarbonylmethyl 5-Aminosulfonyl-4-chloro-2-[(2-furanylmethyl)amino]benzoate (Furosemide N,N-Dimethylglycolamido Ester)

The method of Bundgaard, H., Norgaard, T. and Nielsen, N. M., Int. J. Pharmaceutics, 1988, 42, 217-224, can be employed to prepare N,N-dimethylaminocarbonylmethyl 5-aminosulfonyl-4-chloro-2-[(2-furanylmethyl)amino]benzoate, m.p. 193-194°.

### EXAMPLE 63

### t-Butylcarbonyloxymethyl 5-Aminosulfonyl-4-chloro-2-[(2-furanylmethyl)amino]benzoate (Furosemide Pivaxetil Ester)

The method of Mork, N., Bundgaard, H., Shalmi, M. and Christensen, S., Int. J. Pharmaceutics, 1990, 60, 163-169, can be employed to prepare t-butylcarbonyloxymethyl 5-aminosulfonyl-4-chloro-2-[(2-furanylmethyl)amino]benzoate.

### EXAMPLE 64

### t-Butylcarbonyloxymethyl 5-Aminosulfonyl-4-chloro-2-[(2-furanylmethyl)amino]dithiobenzoate (Furosemide Pivaxetil Dithioester)

In similar manner to Example 63, dithiofurosemide can be reacted with chloromethyl pivalate, triethylamine and sodium iodide in dimethylformamide (DMF) to yield t-butylcarbonyloxymethyl 5-aminosulfonyl-4-chloro-2-[(2-furanylmethyl)amino]dithiobenzoate.

### EXAMPLE 65

### Ethylcarbonyloxymethyl 5-Aminosulfonyl-4-chloro-2-[(2-furanylmethyl)amino]benzoate (Furosemide Propaxetil Ester)

The method of Mork, N., Bundgaard, H., Shalmi, M. and Christensen, S., Int. J. Pharmaceutics, 1990, 60, 163-169, can be employed to prepare ethylcarbonyloxymethyl 5-aminosulfonyl-4-chloro-2-[(2-furanylmethyl)amino]benzoate, m.p. 141-142°.

### EXAMPLE 66

### 5-[1-(t-Butylcarbonyloxymethyl)-1H-tetrazol-5-yl]-2-chloro-4-[(2-thienylmethyl)amino]benzenesulfonamide (Tetrazolyl-Substituted Azosemide)

In similar manner to Example 19, azosemide can be reacted with chloromethyl pivalate, triethylamine and sodium iodide in DMF to yield 5-[1-(t-Butylcarbonyloxymethyl)-1H-tetrazol-5-yl]-2-chloro-4-[(2-thienylmethyl)amino]benzenesulfonamide.

### EXAMPLE 67

### 2-Chloro-5-[1-(ethylcarbonyloxymethyl)-1H-tetrazol-5-yl]-4-[(2-thienylmethyl)amino]benzenesulfonamide (Tetrazolyl-Substituted Azosemide)

In similar manner to Example 19, azosemide can be reacted with chloromethyl propionate, triethylamine and sodium iodide in DMF to yield 2-chloro-5-[1-(ethylcarbonyloxymethyl)-1H-tetrazol-5-yl]-4-[(2-thienylmethyl)amino]benzenesulfonamide.

### EXAMPLE 68

### 2-Chloro-5-[1-(hydroxymethyl)-1H-tetrazol-5-yl]-4-[(2-thienylmethyl)amino]benzenesulfonamide (Tetrazolyl-Substituted Azosemide)

Azosemide can be reacted with formaldehyde in methylene chloride, methylene chloride-DMF mixtures or DMF to yield 2-chloro-5-[1-(hydroxymethyl)-1H-tetrazol-5-yl]-4-[(2-thienylmethyl)amino]benzenesulfonamide.

### EXAMPLE 69

### 2-Chloro-5-[1-(methoxymethyl)-1H-tetrazol-5-yl]-4-[(2-thienylmethyl)amino]benzenesulfonamide (Tetrazolyl-Substituted Azosemide)

Azosemide can be reacted with formaldehyde, methanol and a strong acid in methylene chloride, methylene chloride-DMF mixtures or DMF to yield 2-chloro-5-[1-(methoxymethyl)-1H-tetrazol-5-yl]-4-[(2-thienylmethyl)amino]benzenesulfonamide.

### EXAMPLE 70

### 2-Chloro-5-[1-(methylthiomethyl)-1H-tetrazol-5-yl]-4-[(2-thienylmethyl)amino]benzenesulfonamide (Tetrazolyl-Substituted Azosemide)

Azosemide can be reacted with formaldehyde, methanethiol and a strong acid in methylene chloride, methylene chloride-DMF mixtures or DMF to yield 2-chloro-5-[1-(methylthiomethyl)-1H-tetrazol-5-yl]-4-[(2-thienylmethyl)amino]benzenesulfonamide.

### EXAMPLE 71

### 5-[1-(Benzoyloxymethyl)-1H-tetrazol-5-yl]-2-chloro-4-[(2-thienylmethyl)amino]benzenesulfonamide (Tetrazolyl-Substituted Azosemide)

Azosemide can be reacted with benzyl chloromethyl ether, triethylamine and sodium iodide in DMF to yield 5-[1-(benzyloxymethyl)-1H-tetrazol-5-yl]-2-chloro-4-[(2-thienylmethyl)amino]benzenesulfonamide.

### EXAMPLE 72

### Benzyltrimethylammonium Salt of 2-Chloro-5-(1H-tetrazol-5-yl)-4-[(2-thienylmethyl)amino]benzenesulfonamide (Azosemide Benzyltrimethylammonium Salt)

In similar manner to Example 16, azosemide can be reacted with benzyltrimethylammonium hydroxide in water to yield the benzyltrimethylammonium salt of 2-chloro-5-(1H-tetrazol-5-yl)-4-[(2-thienylmethyl)amino]benzenesulfonamide.

### EXAMPLE 73

### Cetyltrimethylammonium Salt of 2-Chloro-5-(1H-tetrazol-5-yl)-4-[(2-thienylmethyl)amino]benzenesulfonamide (Azosemide Cetyltrimethylammonium Salt)

In similar manner to Example 16, azosemide can be reacted with cetyltrimethylammonium hydroxide in water to yield the cetyltrimethylammonium salt of 2-chloro-5-(1H-tetrazol-5-yl)-4-[(2-thienylmethyl)amino]benzenesulfonamide.

### EXAMPLE 74

### 3-Isopropylcarbamylsulfonamido-4-(3′-methylphenyl)aminopyridinium t-Butylcarbonyloxymethochloride (Pyridinium-Substituted Torsemide Salt)

In similar manner to Example 19, torsemide can be reacted with chloromethyl pivalate, triethylamine and sodium iodide in DMF to yield 3-isopropylcarbamylsulfonamido-4-(3′-methylphenyl)aminopyridinium t-butylcarbonyloxymethochloride and some 3-isopropylcarbamylsulfonamido-4-(3′-methylphenyl)aminopyridinium t-butylcarbonyloxymethoiodide.

### EXAMPLE 75

### 3-Isopropylcarbamylsulfonamido-4-(3′-methylphenyl)aminopyridinium Ethylcarbonyloxymethochloride (Pyridinium-Substituted Torsemide Salt)

In similar manner to Example 19, torsemide can be reacted with chloromethyl propionate, triethylamine and sodium iodide in DMF to yield 3-isopropylcarbamylsulfonamido-4-(3′-methylphenyl)aminopyridinium ethylcarbonyloxymethochloride and some 3-isopropylcarbamylsulfonamido-4-(3′-methylphenyl)aminopyridinium ethylcarbonyloxymethoiodide.

### EXAMPLE 76

### 3-Isopropylcarbamylsulfonamido-4-(3′-methylphenyl)aminopyridinium benzyloxymethochloride (Pyridinium-Substituted Torsemide Salt)

In a similar manner to Example 8, torsemide can be reacted with benzyl chloromethyl ether and triethylamine in DMF to yield 3-isopropylcarbamylsulfonamido-4-(3′-methylphenyl)aminopyridinium benzyloxymethochloride.

### EXAMPLE 77

### 3-Isopropylcarbamylsulfonamido-4-(3′-methylphenyl)aminopyridinium methoxymethochloride (Pyridinium-Substituted Torsemide Salt)

In a similar manner to Example 8, torsemide can be reacted with methyl chloromethyl ether and triethylamine and in DMF to yield 3-isopropylcarbamylsulfonamido-4-(3′-methylphenyl)aminopyridinium methoxymethochloride.

### EXAMPLE 78

### 3-Isopropylcarbamylsulfonamido-4-(3′-methylphenyl)aminopyridinium phenylmethochloride (Pyridinium-Substituted Torsemide Salt)

In a similar manner to Example 8, torsemide can be reacted with benzyl chloride and triethylamine in DMF to yield 3-isopropylcarbamylsulfonamido-4-(3′-methylphenyl)aminopyridinium phenylmethochloride.

### EXAMPLE 79

### 3-Isopropylcarbamylsulfonamido-4-(3′-methylphenyl)aminopyridinium Benzylthiomethochloride (Pyridinium-Substituted Torsemide Salt)

In a similar manner to Example 8, torsemide can be reacted with benzyl chloromethyl thioether and triethylamine in DMF to yield 3-Isopropylcarbamylsulfonamido-4-(3′-methylphenyl)aminopyridinium benzylthiamethochloride.

### EXAMPLE 80

### 3-Isopropylcarbamylsulfonamido-4-(3′-methylphenyl)aminopyridinium Methylthiomethochloride (Pyridinium-Substituted Torsemide Salt)

In a similar manner to Example 8, torsemide can be reacted with methyl chloromethyl thioether and triethylamine and in DMF to yield 3-isopropylcarbamylsulfonamido-4-(3′-methylphenyl)aminopyridinium methylthiamethochloride.

### EXAMPLE 81

Methoxy(polyethyleneoxy)ₙ₋₁-ethyl 3-Aminosulfonyl-5-butylamino-4-phenoxybenzoate (Bumetanide mPEG350 Esters)

In a manner similar to Example 8, bumetanide can be reacted with MeO-PEG350-Cl (Biolink Life Sciences, Inc., Cary, N.C., BLS-106-350) and triethylamine in DMF to yield methoxy(polyethyleneoxy)ₙ₋₁-ethyl 3-aminosulfonyl-5-butylamino-4-phenoxybenzoate where n is in the 7-8 range.

### EXAMPLE 82

### Methoxy(polyethyleneoxy)ₙ₋₁-ethyl 3-Aminosulfonyl-5-butylamino-4-phenoxybenzoate (S-Bumetanide mPEG350 Thioesters)

In a manner similar to Example 8, thiobumetanide can be reacted with MeO-PEG350-Cl (Biolink Life Sciences, Inc., Cary, N.C., BLS-106-350) and triethylamine in DMF to yield methoxy(polyethyleneoxy)ₙ₋₁-ethyl 3-aminosulfonyl-5-butylamino-4-phenoxy-thiobenzoate where n is in the 7-8 range.

### EXAMPLE 83

### Methoxy(polyethyleneoxy)ₙ₋₁-ethyl 3-Aminosulfonyl-5-butylamino-4-phenoxybenzoate (Bumetanide mPEG1000 Esters)

In a manner similar to Example 8, bumetanide can be reacted with MeO-PEG1000-OTs (Biolink Life Sciences, Inc., Cary, N.C., BLS-107-1000) and triethylamine in DMF to yield methoxy(polyethyleneoxy)ₙ₋₁-ethyl 3-aminosulfonyl-5-butylamino-4-phenoxybenzoate where n is in the 19-24 range. In similar manner S-bumetanide mPEG1000 thiosters can be formed with S-thiobumetanide, MeO-PEG1000-OTs (Biolink Life Sciences, Inc., Cary, N.C., BLS-107-1000) and triethylamine in DMF.

### EXAMPLE 84

### Methoxy(polyethyleneoxy)ₙ₋₁-ethyl 3-Aminosulfonyl-S-butylamino-4-phenoxybenzoate (Bumetanide mPEG1000 Dithioesters)

In a manner similar to Example 8, dithiobumetanide can be reacted with MeO-PEG1000-OTs (Biolink Life Sciences, Inc., Cary, N.C., BLS-107-1000) and triethylamine in DMF to yield methoxy(polyethyleneoxy)ₙ₋₁-ethyl 3-aminosulfonyl-5-butylamino-4-phenoxy-dithiobenzoate where n is in the 19-24 range.

### EXAMPLE 85

### Methoxy(polyethyleneoxy)ₙ₋₁-ethyl 3-Aminosulfonyl-4-phenoxy-5-(1-pyrrolidinyl)benzoate (Piretanide mPEG350 Esters)

In similar manner to Example 8, piretanide can be reacted with MeO-PEG350-Cl (Biolink Life Sciences, Inc., Cary, N.C., BLS-106-350) and triethylamine in DMF to yield methoxy(polyethyleneoxy)ₙ₋₁-ethyl 3-aminosulfonyl-4-phenoxy-5-(1-pyrrolidinyl)benzoate where n is in the 7-8 range. In similar manner bumetanide mPEG350 dithiosters can be formed with dithiobumetanide, MeO-PEG350-Cl (Biolink Life Sciences, Inc., Cary, N.C. BLS-106-350) and triethylamine in DMF.

### EXAMPLE 86

### Methoxy(polyethyleneoxy)ₙ₋₁-ethyl 3-Aminosulfonyl-4-phenoxy-5-(1-pyrrolidinyl)benzoate (S-Piretanide mPEG350 Thioesters)

In similar manner to Example 8, thiopiretanide can be reacted with MeO-PEG350-Cl (Biolink Life Sciences, Inc., Cary, N.C., BLS-106-350) and triethylamine in DMF to yield methoxy(polyethyleneoxy)ₙ₋₁-ethyl 3-aminosulfonyl-4-phenoxy-5-(1-pyrrolidinyl)thiobenzoate where n is in the 7-8 range.

### EXAMPLE 87

### Methoxy(polyethyleneoxy)ₙ₋₁-ethyl 3-Aminosulfonyl-4-phenoxy-5-(1-pyrrolidinyl)benzoate (Piretanide mPEG1000 Esters)

In similar manner to Example 8, piretanide can be reacted with MeO-PEG1000-OTs (Biolink Life Sciences, Inc., Cary, N.C., BLS-107-1000) and triethylamine in DMF to yield methoxy(polyethyleneoxy)ₙ₋₁-ethyl 3-aminosulfonyl-4-phenoxy-5-(1-pyrrolidinyl)benzoate where n is in the 19-24 range. In similar manner S-piretanide mPEG1000 thiosters can be formed with S-thiopiretanide, MeO-PEG1000-OTs (Biolink Life Sciences, Inc., Cary, N.C., BLS-107-1000) and triethylamine in DMF.

### EXAMPLE 88

### Methoxy(polyethyleneoxy)ₙ₋₁-ethyl 3-Aminosulfonyl-4-phenoxy-5-(1-pyrrolidinyl)benzoate (Piretanide mPEG1000 Dithioesters)

In similar manner to Example 8, dithiopiretanide can be reacted with MeO-PEG1000-OTs (Biolink Life Sciences, Inc., Cary, N.C., BLS-107-1000) and triethylamine in DMF to yield methoxy(polyethyleneoxy)ₙ₋₁-ethyl 3-aminosulfonyl-4-phenoxy-5-(1-pyrrolidinyl)dithiobenzoate where n is in the 19-24 range. In similar manner piretanide mPEG1000 dithiosters can be formed with dithiopiretanide, MeO-PEG1000-OTs (Biolink Life Sciences, Inc., Cary, N.C., BLS-107-1000) and triethylamine in DMF.

### EXAMPLE 89

### Methoxy(polyethyleneoxy)ₙ₋₁-ethyl 5-Aminosulfonyl-4-chloro-2-[(2-furanylmethyl)amino]benzoate (Furosemide mPEG350 Esters)

In similar manner to Example 8, furosemide can be reacted with MeO-PEG350-Cl (Biolink Life Sciences, Inc., Cary, N.C., BLS-106-350) and triethylamine in DMF to yield methoxy(polyethyleneoxy)ₙ₋₁-ethyl 5-aminosulfonyl-4-chloro-2-[(2-furanylmethyl)amino]benzoate where n is in the 7-8 range.

### EXAMPLE 90

### Methoxy(polyethyleneoxy)ₙ₋₁-ethyl 5-Aminosulfonyl-4-chloro-2-[(2-furanylmethyl)amino]benzoate (S-Furosemide mPEG350 Thioesters)

In similar manner to Example 8, thiofurosemide can be reacted with MeO-PEG350-Cl (Biolink Life Sciences, Inc., Cary, N.C., BLS-106-350) and triethylamine in DMF to yield methoxy(polyethyleneoxy)ₙ₋₁-ethyl 5-aminosulfonyl-4-chloro-2-[(2-furanylmethyl)amino]thiobenzoate where n is in the 7-8 range.

### EXAMPLE 91

### Methoxy(polyethyleneoxy)ₙ₋₁-ethyl 5-Aminosulfonyl-4-chloro-2-[(2-furanylmethyl)amino]benzoate (Furosemide mPEG1000 Esters)

In similar manner to Example 8, furosemide can be reacted with MeO-PEG1000-OTs (Biolink Life Sciences, Inc., Cary, N.C., BLS-107-1000) and triethylamine in DMF to yield methoxy(polyethyleneoxy)ₙ₋₁-ethyl 5-aminosulfonyl-4-chloro-2-[(2-furanylmethyl)amino]benzoate where n is in the 19-24 range.

### EXAMPLE 92

### Methoxy(polyethyleneoxy)ₙ₋₁-ethyl 5-Aminosulfonyl-4-chloro-2-[(2-furanylmethyl)amino]benzoate (Furosemide mPEG1000 Dithioesters)

In similar manner to Example 8, dithiofurosemide can be reacted with MeO-PEG1000-OTs (Biolink Life Sciences, Inc., Cary, N.C., BLS-107-1000) and triethylamine in DMF to yield methoxy(polyethyleneoxy)ₙ₋₁-ethyl 5-aminosulfonyl-4-chloro-2-[(2-furanylmethyl)amino]dithiobenzoate where n is in the 19-24 range. In similar manner furosemide mPEG350 dithiosters can be formed with dithiofurosemide, MeO-PEG350-Cl (Biolink Life Sciences, Inc., Cary, N.C., BLS-106-350) and triethylamine in DMF.

### EXAMPLE 93

### 5-[1-[Methoxy(polyethyleneoxy)ₙ₋₁-ethyl]-1H-tetrazol-5-yl]-2-chloro-4-[(2-thienylmethyl)amino]benzenesulfonamides (N-mPEG350-Tetrazolyl-Substituted Azosemides)

In similar manner to Example 8, azosemide can be reacted with MeO-PEG350-CI (Biolink Life Sciences. Inc., Cary, N.C., BLS-106-350) and triethylamine in DMF to yield 5-[1-[methoxy(polyethyleneoxy)ₙ₋₁-ethyl]-1H-tetrazol-5-yl]-2-chloro-4-[(2-thienylmethyl)amino]benzenesulfonamides where n is in the 7-8 range.

### EXAMPLE 94

### 5-[1-[Methoxy(polyethyleneoxy)ₙ₋₁-ethyl]-1H-tetrazol-5-yl]-2-chloro-4-[(2-thienylmethyl)amino]benzenesulfonamides (N-mPEG1000-Tetrazolyl-Substituted Azosemides)

In similar manner to Example 8, azosemide can be reacted with MeO-PEG1000-OTs (Biolink Life Sciences, Inc., Cary, N.C., BLS-107-1000) and triethylamine in DMF to yield 5-[1-[methoxy(polyethyleneoxy)ₙ₋₁-ethyl]-1H-tetrazol-5-yl]-2-chloro-4-[(2-thienylmethyl)amino]benzenesulfonamides where n is in the 19-24 range.

### EXAMPLE 95

### 3-Isopropylcarbamylsulfonamido-4-(3′-methylphenyl)aminopyridinium Methoxy(polyethyleneoxy)ₙ₋₁-ethochlorides (N-mPEG350-Pyridinium Torsemide Salts)

In similar manner to Example 8, torsemide can be reacted with MeO-PEG350-Cl (Biolink Life Sciences, Inc., Cary, N.C., BLS-106-350) and triethylamine in DMF to yield 3-isopropylcarbamylsulfonamido-4-(3′-methylphenyl)aminopyridinium methoxy(polyethyleneoxy)ₙ₋₁-ethochlorides where n is in the 7-8 range.

### EXAMPLE 96

### 3-Isopropylcarbamylsulfonamido-4-(3′-methylphenyl)aminopyridinium Methoxy(polyethyleneoxy)ₙ₋₁-ethochlorides (N-mPEG1000-Pyridinium Torsemide Salts)

In similar manner to Example 8, torsemide can be reacted with MeO-PEG1000-OTs (Biolink Life Sciences, Inc., Cary, N.C., BLS-107-1000) and triethylamine in DMF to yield 3-isopropylcarbamylsulfonamido-4-(3′-methylphenyl)aminopyridinium methoxy(polyethyleneoxy)ₙ₋₁-ethochlorides where n is in the 19-24 range.

### EXAMPLE 97

### 3-Aminosulfonyl-5-butylamino-4-phenoxybenzaldehyde (Bumetanide Aldehyde)

By the method of Muraki and Mukiayama (Chem. Letters, 1974, 1447 and Chem. Letters, 1975, 215), bumetanide can be reacted with bis(4-methylpiperazinyl)aluminum hydride to yield 3-aminosulfonyl-5-butylamino-4-phenoxybenzaldehyde.

### EXAMPLE 98

### 3-Aminosulfonyl-4-phenoxy-5-(1-pyrrolidinyl)benzaldehyde (Piretanide Aldehyde)

By the method of Muraki and Mukiayama (Chem. Letters, 1974, 1447 and Chem. Letters, 1975, 215), piretanide can be reacted with bis(4-methylpiperazinyl)aluminum hydride to yield 3-aminosulfonyl-4-phenoxy-5-(1-pyrrolidinyl)benzaldehyde.

### EXAMPLE 99

### 5-Aminosulfonyl-4-chloro-2-[(2-furanylmethyl)amino]benzaldehyde (Furosemide Aldehyde)

By the method of Muraki and Mukiayama (Chem. Letters, 1974, 1447 and Chem. Letters, 1975, 215), furosemide can be reacted with bis(4-methylpiperazinyl)aluminum hydride to yield 5-aminosulfonyl-4-chloro-2-[(2-furanylmethyl)amino]benzaldehyde.

### EXAMPLE 100

### Assessment of the Therapeutic Potential of Bumetanide Analogs in Alleviating Anxiety (Fear Potentiated Startle Paradigm)

Purpose:

To evaluate the effects of bumetanide analogs in two tests of anxiety in rats. Bumetanide analogs (bumetanide 3-(dimethylaminopropyl) ester, bumetanide benzyltrimethylammonium salt, bumetanide dibenzylamide, bumetanide cyanomethyl ester, bumetanide N,N-diethylglycolamido ester, bumetanide N,N-dimethylglycolamido ester, bumetanide morpholinoethyl ester, bumetanide pivaxetil ester, bumetanide methyl ester, bumetanide diethylamide and benzyl ester) were assessed in the fear potentiated startle paradigm (FPS) test of anxiety. These studies may be repeated using furosemide analogs, piretanide analogs, azosemide analogs and torsemide analogs.

FPS Design:

FPS is a commonly used assessment of the therapeutic value of anxiolytic compounds in the rat. Rats received a 30 min period of habituation to the FPS apparatus. 24-hr later baseline startle amplitudes were collected. The rats will be divided into two matched groups based on baseline startle amplitudes. Following baseline startle amplitude collection 20 light/shock pairings were delivered on 2 sessions over 2 consecutive days (i.e., 10 light/shock pairings per day). On the final day, one group of rats received an injection (i.v.) of a bumetanide analog and the other group received vehicle. Immediately following injections, startle amplitudes were assessed during startle alone trials and startle plus fear (light followed by startle) trials. Fear potentiated startle (light+startle amplitudes minus startle alone amplitudes) was compared between the treatment groups.

Method:

Fear Potentiated Startle

Animals were trained and tested in four identical stabilimeter devices (Med-Associates). Briefly, each rat was placed in a small Plexiglas cylinder. The floor of each stabilimeter consists of four 6-mm-diameter stainless steel bars spaced 18 mm apart through which shock can be delivered. Cylinder movements result in displacement of an accelerometer where the resultant voltage is proportional to the velocity of the cage displacement. Startle amplitude is defined as the maximum accelerometer voltage that occurs during the first 0.25 sec after the startle stimulus is delivered. The analog output of the accelerometer is amplified, digitized on a scale of 0-4096 units and stored on a microcomputer. Each stabilimeter is enclosed in a ventilated, light-, and sound-attenuating box. All sound level measurements were made with a Precision Sound Level Meter. The noise of a ventilating fan attached to a sidewall of each wooden box produces an overall background noise level of 64 dB. The startle stimulus is a 50 ms burst of white noise (5 ms risedecay time) generated by a white noise generator. The visual conditioned stimulus used was illumination of a light bulb adjacent to the white noise source. The unconditioned stimulus was a 0.6 mA foot shock with duration of 0.5 sec, generated by four constant-current shockers located outside the chamber. The presentation and sequencing of all stimuli were under the control of the microcomputer.

FPS procedures consisted of 5 days of testing; during days 1 and 2 baseline startle responses were collected, days 3 and 4 light/shock pairings were delivered, day testing for fear potentiated startle was conducted.

Matching. On the first two days all rats were placed in the Plexiglas cylinders and 3 min later presented with 30 startle stimuli at a 30 sec interstimulus interval. An intensity of 105 dB was used. The mean startle amplitude across the 30 startle stimuli on the second day was used to assign rats into treatment groups with similar means.

Training. On the following 2 days, rats were placed in the Plexiglas cylinders. Each day following 3 min after entry 10 CS-shock pairings were delivered. The shock was delivered during the last 0.5 sec of the 3.7 sec CSs at an average intertrial interval of 4 min (range, 3-5 min).

Testing. Rats were placed in the same startle boxes where they were trained and after 3 min were presented with 13 startle-eliciting stimuli (all at 105 dB). These initial startle stimuli were used to again habituate the rats to the acoustic startle stimuli. Thirty seconds after the last of these stimuli, each animal received 60 startle stimuli with half of the stimuli presented alone (startle alone trials) and the other half presented 3.2 sec after the onset of the 3.7 sec CS (CS-startle trials). All startle stimuli were presented at a mean 30 sec interstimulus interval, randomly varying between 20 and 40 sec.

Measures. The treatment groups were compared on the difference in startle amplitude between CS-startle and startle-alone trials (fear potentiation).

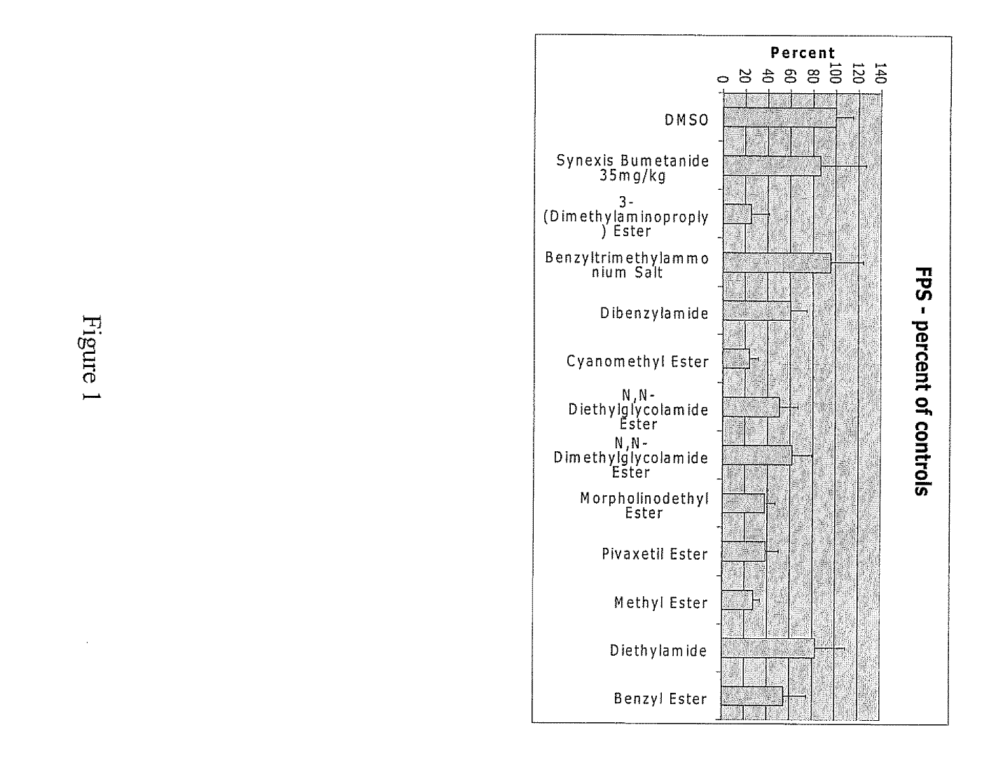

In general, this study showed the ability of bumetanide analogs of the present invention to traverse the blood-brain barrier. The bumetanide analogs show the potential for regulation of CNS disorders where bumetanide analogs were shown to affect the startle amplitude where the greater the reduction in fear-potentiated startle, the more compound believed delivered to the CNS. Moreover, several bumetanide analogs were shown to be more potent or at least as potent as bumetanide. See Table 1 below and FIG. 1.

TABLE 1
Number of animals

| Compound | N |
|---|---|
| DMSO | 34 |
| Sigma Bumetanide 35 mg/kg | 6 |
| Sigma Bumetanide 40 mg/kg | 6 |
| Synexis Bumetanide 35 mg/kg | 11 |
| 3-(Dimethylaminoproply) | 17 |
| Ester | |
| Benzyltrimethylammonium | 12 |
| Salt | |
| Dibenzylamide | 15 |
| Cyanomethyl Ester | 17 |
| N,N-Diethylglycolamide Ester | 17 |
| N,N-Dimethylglycolamide | 17 |
| Ester | |
| Morpholinodethyl Ester | 17 |
| Pivaxetil Ester | 17 |
| Methyl Ester | 17 |
| Diethylamide | 12 |
| Benzyl Ester | 12 |
| Total | 227 |

### EXAMPLE 101

### Assessment of the Therapeutic Potential of Bumetanide, Furosemide, Piretanide, Azosemide and Torsemide Analogs in Alleviating the Symptoms of Intense Anxiety or Post Traumatic Stress Disorder (Contextual Fear Conditioning Model)

Purpose:

To evaluate the potential of bumetanide, furosemide, piretanide, azosemide and torsemide analogs to alleviate intense anxiety in contextual fear conditioning in rats.

Design:

Contextual fear conditioning involves pairing an aversive event, in this case moderate foot shock, with a distinctive environment. The strength of the fear memory is assessed using freezing, a species-typical defensive reaction in rats, marked by complete immobility, except for breathing. If rats are placed into a distinctive environment and are immediately shocked they do not learn to fear the context. However, if they are allowed to explore the distinctive environment sometime before the immediate shock, they show intense anxiety and fear when placed back into the same environment. We can take advantage of this fact, and by procedurally dividing contextual fear conditioning into two phases, we can separately study effects of treatments on memory for the context (specifically a hippocampus based process) from learning the association between context and shock or experiencing the aversiveness of the shock (which depend upon emotional response circuitry including amygdala). PTSD in humans has been shown to be related to emotional response circuitry in the amygdala, for this reason contextual memory conditioning is a widely accepted model for PTSD.

The experiment will use 24 rats. Each rat will receive a single 5-min episode of exploration of a small, novel environment. 72-hr later they will be placed into the same environment and immediately they will receive a single, moderate foot-shock. 24-hr later, 12 of the rats will receive an injection (I.V) of a bumetanide analog. The remaining 12 rats will receive an injection of the vehicle. Each rat will again be placed into the same environment for 8-min during which time freezing will be measured, as an index of Pavlovian conditioned fear.

Methods:

In this experiment, we 4 identical chambers (20×20×15 cm) are used. All aspects of the timing and control of events are under microcomputer control (MedPC, MedAssociates Inc, Vermont, USA). Measurement of freezing is accomplished through an overhead video camera connected to the microcomputer and is automatically scored using a specialty piece of software, FreezeFrame. In Phase 1, rats are placed individually into the chambers for 5 minutes. Phase 2 begins 72 hr later, when again rats are placed individually into the same chambers but they receive an immediate foot shock (1 mA for 2 s). Thirty seconds later they are removed from the chambers. Phase 3, 24 hr later, the rats are returned to the chambers for 8 min during which time we score freezing, our index of conditioning fear. Total freezing time will be analyzed in a one-way ANOVA with drug dose as the within-groups factor.

### EXAMPLE 102

### Formulations for CNS-Targeted Drugs

A. Oral Preparations

For oral administration, the pharmaceutical components are used in the range of about 10-60 mg of drug substance together with various inactive ingredients such as microcrystalline cellulose and other excipients, contained in a gelatin capsule. Alternatively, the drug substance is provided in tablet form including about 10-60 mg, of drug substance with microcrystalline cellulose, hydroxypropyl cellulose, magnesium stearate and other excipients.

B. Intravenous Preparations

For intravenous administration, each milliliter of sterile solution can include about 1-25 mg of drug substance formulated with about 20-40% propylene glycol, about 0-10% ethyl alcohol, optionally water, buffers, for example, about 5% sodium benzoate and benzoic acid as buffers, and preservatives, for example, about 1.5% benzyl alcohol as a preservative.

The foregoing is illustrative of the present invention, and is not to be construed as limiting thereof. The invention is defined by the following claims, with equivalents of the claims to be included therein.

That which is claimed is:

1. A compound selected from the group consisting of the following: 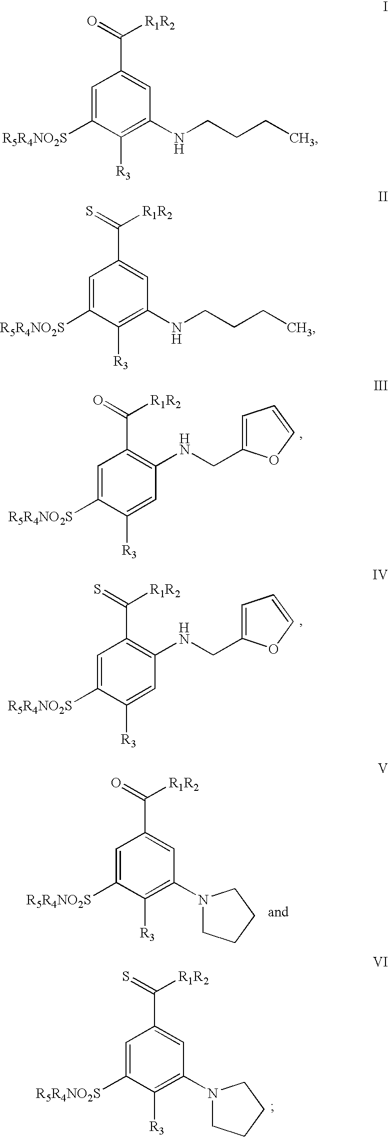
or a pharmaceutically acceptable salt, solvate, tautomer or hydrate thereof, wherein
R₁ is not present, H, O or S;
R₂ is not present, H or when R₁ is O or S, R₂ is selected from the group consisting of hydrogen, alkyl, aralkyl, aryl, alkylaminodialkyl, alkylcarbonylaminodialkyl, alkyloxycarbonylalkyl, alkylcarbonyloxyalkyl, alkylaldehyde, alkylketoalkyl, alkylamide, alkarylamide, acrylamide, an alkylammonium group, alkylcarboxylic acid, alkylheteroaryl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, and when R₁ is not present, R₂ is selected from the group consisting of hydrogen, N,N-dialkylamino, N,N-dialkarylamino, N,N-diarylamino, N-alkyl-N-alkarylamino, N-alkyl-N-arylamino, N-alkaryl-N-arylamino, unsubstituted or substituted;
R₃ is selected from the group consisting of aryl, halo, hydroxy, alkoxy, and aryloxy, unsubstituted or substituted; and
R₄ and R₅ are each independently selected from the group consisting of hydrogen, alkylaminodialkyl, carbonylalkyl, carbonylalkaryl, carbonylaryl and salts thereof, with the following provisos:
R₃ of formula I is not phenyloxy when R₁ is O and R₂, R₄ and R₅ are H;
R₃ of formula III is not Cl, when R₁ is O and R₂ and R₅ are H;
R₂ of formula III is not methyl when R₁ is O, R₃ is Cl, and R₄ and R₅ are H; and
R₃ of formula V is not phenyloxy when R₁ is O and R₂, R₄ and R₅ are H.

2. The compound of claim 1, wherein the compound is selected from the group consisting of bumetanide aldehyde, bumetanide methyl ester, bumetanide cyanomethyl ester, bumetanide ethyl ester, bumetanide isoamyl ester, bumetanide octyl ester, bumetanide benzyl ester, bumetanide dibenzylamide, bumetanide diethylamide, bumetanide morpholinoethyl ester, bumetanide 3-(dimethylaminopropyl) ester, bumetanide N,N-diethylglycolamide ester, bumetanide N,N-dimethylglycolamidoe ester, bumetanide pivaxetil ester, bumetanide propaxetil ester, bumetanide methoxy(polyethyleneoxy)ₙ₋₁-ethyl ester, bumetanide benzyltrimethylammonium salt and bumetanide cetyltrimethylammonium salt.

3. The compound of claim 1, wherein the compound is selected from the group consisting of bumetanide S-methyl thioester, bumetanide S-cyanomethyl thioester, bumetanide S-ethyl thioester, bumetanide S-isoamyl thioester, bumetanide S-octyl thioester, bumetanide S-benzyl thioester, bumetanide S-(morpholinoethyl) thioester, bumetanide S-[3-(dimethylaminopropyl)]thioester, bumetanide S-(N,N)-diethylglycolamido) thioester, bumetanide S-(N,N-dimethylglycolamido) thioester, bumetanide S-pivaxetil thioester, bumetanide S-propaxetil thioester, bumetanide S-methoxy(polyethyleneoxy)ₙ₋₁-ethyl thioester, bumetanide thioacid (thiobumetanide), bumetanide S-benzyltrimethylammonium S-thioacid salt and bumetanide S-cetyltrimethylammonium thioacid salt.

4. The compound of claim 1, wherein the compound is selected from the group consisting of metastable bumetanide thioacid, bumetanide O-methyl thioester, bumetanide O-cyanomethyl thioester, bumetanide O-ethyl thioester, bumetanide O-isoamyl thioester, bumetanide O-octyl thioester, bumetanide O-benzyl thioester, bumetanide O-(morpholinoethyl) thioester, bumetanide O-[3-(dimethylaminopropyl)]thioester, bumetanide O-(N,N-diethylglycolamido) thioester, bumetanide, O-(N,N-dimethylglycolamido) thioester, bumetanide O-pivaxetil thioester, bumetanide O-propaxetil thioester, bumetanide O-[methoxy(polyethyleneoxy)ₙ₋₁-ethyl]thioester, bumetanide O-benzyltrimethylammonium thioacid salt and bumetanide O-cetyltrimethylammonium thioacid salt.

5. The compound of claim 1, wherein the compound is selected from the group consisting of bumetanide thioaldehyde, bumetanide dithioacid, bumetanide methyl dithioester, bumetanide cyanomethyl dithioester, bumetanide ethyl dithioester, bumetanide isoamyl dithioester, bumetanide octyl dithioester, bumetanide benzyl dithioester, bumetanide dibenzylthioamide, bumetanide diethylthioamide, bumetanide morpholinoethyl dithioester, bumetanide 3-(dimethylaminopropyl) dithioester, bumetanide N,N-diethylglycolamido dithioester, bumetanide N,N-dimethylglycolamido dithioester, bumetanide pivaxetil dithioester, bumetanide propaxetil dithioester, bumetanide methoxy(polyethyleneoxy)ₙ₋₁-ethyl dithioester, bumetanide benzyltrimethylammonium dithioacid salt and bumetanide cetyltrimethylammonium dithioacid salt.

6. The compound of claim 1, wherein the compound is selected from the group consisting of furosemide methyl ester, furosemide cyanomethyl ester, furosemide ethyl ester, furosemide isoamyl ester, furosemide octyl ester, furosemide benzyl ester, furosemide morpholinoethyl ester, furosemide 3-(dimethylaminopropyl) ester, furosemide N,N-diethylglycolamido ester, furosemide N,N-dimethylglycolamido ester, furosemide pivaxetil ester, furosemide propaxetil ester, furosemide methoxy(polyethyleneoxy)ₙ₋₁-ethyl ester, furosemide benzyltrimethylammonium acid salt and furosemide cetyltrimethylammonium acid salt.

7. The compound of claim 1, wherein the compound is selected from the group consisting of furosemide S-thioacid, furosemide S-methyl thioester, furosemide S-cyanomethyl thioester, furosemide S-ethyl thioester, furosemide S-isoamyl thioester, furosemide S-octyl thioester, furosemide S-benzyl thioester, furosemide S-(morpholinoethyl) thioester, furosemide S-[3-(dimethylaminopropyl)]thioester, furosemide S-N,N-diethylglycolamido) thioester, furosemide S-(N,N-dimethylglycolamido) thioester, furosemide S-pivaxetil thioester, furosemide S-propaxetil thioester, furosemide S-[methoxy(polyethyleneoxy)ₙ₋₁-ethyl]thioester, furosemide S-benzyltrimethylammonium thioacid salt, and furosemide S-cetyltrimethylammonium thioacid salt.

8. The compound of claim 1, wherein the compound is selected from the group consisting of metastable furosemide thioacid, furosemide O-methyl thioester, furosemide O-cyanomethyl thioester, furosemide O-ethyl thioester, furosemide O-isoamyl thioester, furosemide O-octyl thioester, furosemide O-benzyl thioester, furosemide O-(morpholinoethyl) thioester, furosemide O-[3-(dimethylaminopropyl)]thioester, furosemide O-(N,N-diethylglycolamido) thioester, furosemide O-(N,N-dimethylglycolamido) thioester, furosemide O-pivaxetil thioester, furosemide O-propaxetil thioester, furosemide O-[methoxy(polyethyleneoxy)ₙ₋₁-ethyl]thioester, furosemide O-benzyltrimethylammonium thioacid salt and furosemide O-cetyltrimethylammonium thioacid salt.

9. The compound of claim 1, wherein the compound is selected from the group consisting of furosemide thioaldehyde, furosemide dithioacid, furosemide methyl dithioester, furosemide cyanomethyl dithioester, furosemide ethyl dithioester, furosemide isoamyl dithioester, furosemide octyl dithioester, furosemide benzyl dithioester, furosemide dibenzylthioamide, furosemide diethylthioamide, furosemide morpholinoethyl dithioester, furosemide 3-(dimethylaminopropyl) dithioester, furosemide N,N-diethylglycolamido dithioester, furosemide N,N-dimethylglycolamido dithioester, furosemide pivaxetil dithioester, furosemide propaxetil dithioester, furosemide methoxy(polyethyleneoxy)ₙ₋₁-ethyl dithioester, furosemide benzyltrimethylammonium dithioacid salt and furosemide cetyltrimethylammonium dithioacid salt.

10. The compound of claim 1, wherein the compound is selected from the group consisting of piretanide aldehyde, piretanide methyl ester, piretanide cyanomethyl ester, piretanide ethyl ester, piretanide isoamyl ester, piretanide octyl ester, piretanide benzyl ester, piretanide dibenzylamide, piretanide diethylamide, piretanide morpholinoethyl ester, piretanide 3-(dimethylaminopropyl) ester, piretanide N,N-diethylglycolamide ester, piretanide dimethylglycolamide ester, piretanide pivaxetil ester, piretanide propaxetil ester, piretanide methoxy(polyethyleneoxy)ₙ₋₁-ethyl ester, piretanide benzyltrimethylammonium salt and piretanide cetyltrimethylammonium salt.

11. The compound of claim 1, wherein the compound is selected from the group consisting of piretanide S-thioacid, piretanide S-methyl thioester, piretanide S-cyanomethyl thioester, piretanide S-ethyl thioester, piretanide S-isoamyl thioester, piretanide S-octyl thioester, piretanide S-benzyl thioester, piretanide S-(morpholinoethyl) thioester, piretanide S-[3-(dimethylaminopropyl)]thioester, piretanide S-N,N-diethylglycolamido) thioester, piretanide S-(N,N-dimethylglycolamido) thioester, piretanide S-pivaxetil thioester, piretanide S-propaxetil thioester, piretanide S-[methoxypolyethyleneoxy)ₙ₋₁-ethyl]thioester, piretanide S-benzyltrimethylammonium thioacid salt and piretanide S-cetyltrimethylammonium thioacid salt.

12. The compound of claim 1, wherein the compound is selected from the group consisting of metastable piretanide O-thioacid, piretanide O-methyl thioester, piretanide O-cyanomethyl thioester, piretanide O-ethyl thioester, piretanide O-isoamyl thioester, piretanide O-octyl thioester, piretanide O-benzyl thioester, piretanide O-(morpholinoethyl) thioester, piretanide O-[3-(dimethylaminopropyl)]thioester, piretanide O-(N,N-diethylglycolamido) thioester, piretanide, O-(N,N-dimethylglycolamido) thioester, piretanide O-pivaxetil thioester, piretanide O-propaxetil thioester, piretanide O-[methoxy(polyethyleneoxy)ₙ₋₁-ethyl]thioester, piretanide O-benzyltrimethylammonium thioacid salt and piretanide O-cetyltrimethylammonium thioacid salt.

13. The compound of claim 1, wherein the compound is selected from the group consisting of piretanide thioaldehyde, piretanide dithioacid, piretanide methyl dithioester, piretanide cyanomethyl dithioester, piretanide ethyl dithioester, piretanide isoamyl dithioester, piretanide octyl dithioester, piretanide benzyl dithioester, piretanide dibenzylthioamide, piretanide diethylthioamide, piretanide morpholinoethyl dithioester, piretanide 3-(dimethylaminopropyl) dithioester, piretanide N,N-diethylglycolamido dithioester, piretanide N,N-dimethylglycolamido dithioester, piretanide pivaxetil dithioester, piretanide propaxetil dithioester, piretanide methoxy(polyethyleneoxy)ₙ₋₁-ethyl dithioester, piretanide benzyltrimethylammonium dithioacid salt and piretanide cetyltrimethylammonium dithioacid salt.

14. A compound of formula VII: 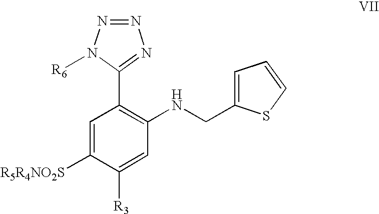
or a pharmaceutically acceptable salt, solvate, tautomer or hydrate thereof, wherein
R₃ is selected from the group consisting of aryl, halo, hydroxy, alkoxy, and aryloxy, unsubstituted or substituted:
R₄ and R₅ are each independently selected from the group consisting of hydrogen, alkylaminodialkyl, carbonylalkyl, carbonylalkaryl, carbonylaryl and salts thereof;
R₆ is selected from the group consisting of alkyloxycarbonylalkyl, alkylaminocarbonylalkyl, alkylaminodialkyl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, with the proviso that R₃ is not Cl, when R₄, R₅ and R₆ are H.

15. The compound of claim 14, wherein the compound is selected from the group consisting of tetrazolyl-substituted azosemide, azosemide benzyltrimethylammonium salt and azosemide cetyltrimethylammonium salt.

16. A compound of formula VIlI: 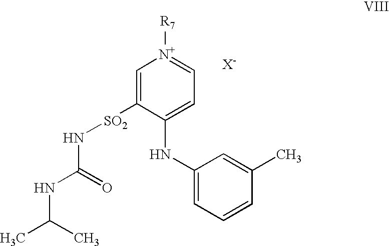
or a pharmaceutically acceptable salt, solvate, tautomer or hydrate thereof,
wherein
R₇ is selected from the group consisting of hydrogen, alkyloxycarbonylalkyl, alkylaminocarbonylalkyl, alkylaminodialkyl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted; and
X⁻ is a halide or an anionic moiety; or alternatively, X⁻ is not present.

17. The compound of claim 16, wherein the compound is a pyridine-substituted torsemide quaternary ammonium salt.

18. A compound selected from the group consisting of S-bumetanide thioacid, O-bumetanide thioacid, bumetanide dithioacid, S-furosemide thioacid, O-furosemide thioacid, furosemide dithioacid, S-piretanide thioacid, O-piretanide thioacid and piretanide dithioacid.

19. A prodrug capable of passage across the blood-brain barrier comprising a compound selected from the group consisting of the following: 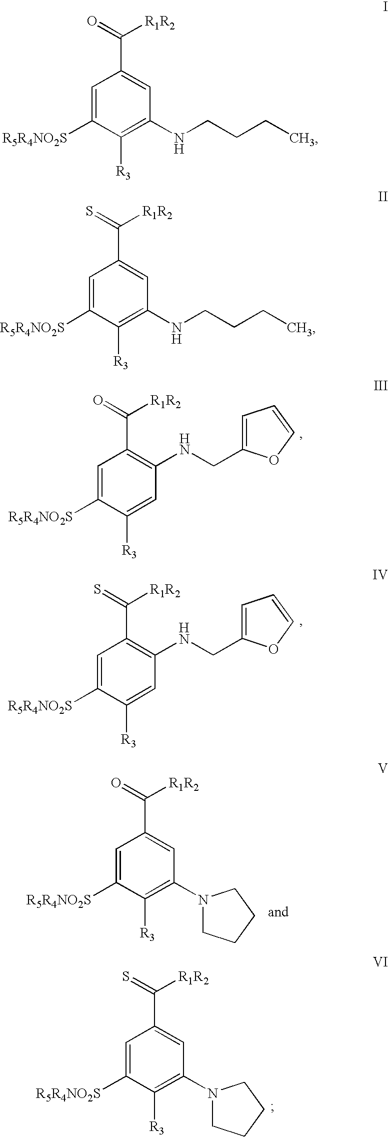
or an ester, pharmaceutically acceptable salt, solvate, tautomer or hydrate thereof, wherein R₁ is not present, H, O or S;
R₂ is not present, H or when R₁ is O or S, R₂ is selected from the group consisting of hydrogen, alkyl, aralkyl, aryl, alkylaminodialkyl, alkylcarbonylaminodialkyl, alkyloxycarbonylalkyl, alkylcarbonyloxyalkyl, alkylaldehyde, alkylketoalkyl, alkylamide, alkarylamide, arylamide, an alkylammonium group, alkylcarboxylic acid, alkylheteroaryl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, and when R₁ is not present, R₂ is selected from the group consisting of hydrogen, N,N-dialkylamino, N,N-dialkarylamino, N,N-diarylamino, N-alkyl-N-alkarylamino, N-alkyl-N-arylamino, N-alkaryl-N-arylamino, unsubstituted or substituted;
R₃ is selected from the group consisting of aryl, halo, hydroxy, alkoxy, and aryloxy, unsubstituted or substituted; and
R₄ and R₅ are each independently selected from the group consisting of hydrogen, alkylaminodialkyl, carbonylalkyl, carbonylalkaryl, carbonylaryl and salts thereof.

20. The prodrug of claim 19, wherein the compound is selected from the group consisting of bumetanide, bumetanide aldehyde, bumetanide methyl ester, bumetanide cyanomethyl ester, bumetanide ethyl ester, bumetanide isoamyl ester, bumetanide octyl ester, bumetanide benzyl ester, bumetanide dibenzylamide, bumetanide diethylamide, bumetanide morpholinoethyl ester, bumetanide 3-(dimethylaminopropyl) ester, bumetanide N,N-diethylglycolamido ester, bumetanide N,N-dimethylglycolamido ester, bumetanide pivaxetil ester, bumetanide propaxetil ester, bumetanide methoxy(polyethyleneoxy)ₙ₋₁-ethyl ester, bumetanide benzyltrimethylammonium salt and bumetanide cetyltrimethylammonium salt.

21. The prodrug of claim 19, wherein the compound is selected from the group consisting of bumetanide S-methyl thioester, bumetanide S-cyanomethyl thioester, bumetanide S-ethyl thioester, bumetanide S-isoamyl thioester, bumetanide S-octyl thioester, bumetanide S-benzyl thioester, bumetanide S-(morpholinoethyl) thioester, bumetanide S-[3-(dimethylaminopropyl)]thioester, bumetanide S-(N,N-diethylglycolamido) thioester, bumetanide S-N,N-dimethylglycolamido) thioester, bumetanide S-pivaxetil thioester, bumetanide S-propaxetil thioester, bumetanide S-methoxy(polyethyleneoxy)ₙ₋₁-ethyl thioester, bumetanide S-thioacid (thiobumetanide), bumetanide S-benzyltrimethylammonium thioacid salt and S-bumetanide cetyltrimethylammonium thioacid salt.

22. The prodrug of claim 19, wherein the compound is selected from the group consisting of metastable bumetanide thioacid, bumetanide O-methyl thioester, bumetanide O-cyanomethyl thioester, bumetanide O-ethyl thioester, bumetanide O-isoamyl thioester, bumetanide O-octyl thioester, bumetanide O-benzyl thioester, bumetanide O-(morpholinoethyl) thioester, bumetanide O-[3-(dimethylaminopropyl)]thioester, bumetanide O-(N,N-diethylglycolamido) thioester, bumetanide, O-(N,N-dimethylglycolamido) thioester, bumetanide O-pivaxetil thioester, bumetanide O-propaxetil thioester, bumetanide O-[methoxy(polyethyleneoxy)ₙ₋₁-ethyl]thioester, bumetanide O-benzyltrimethylammonium thioacid salt and O-bumetanide cetyltrimethylammonium thioacid salt.

23. The prodrug of claim 19, wherein the compound is selected from the group consisting of bumetanide thioaldehyde, bumetanide dithioacid, bumetanide methyl dithioester, bumetanide cyanomethyl dithioester, bumetanide ethyl dithioester, bumetanide isoamyl dithioester, bumetanide octyl dithioester, bumetanide benzyl dithioester, bumetanide dibenzylthioamide, bumetanide diethylthioamide, bumetanide morpholinoethyl dithioester, bumetanide 3-(dimethylaminopropyl) dithioester, bumetanide N,N-diethylglycolamido dithioester, bumetanide N,N-dimethylglycolamido dithioester, bumetanide pivaxetil dithioester, bumetanide propaxetil dithioester, bumetanide methoxy(polyethyleneoxy)ₙ₋₁-ethyl dithioester, bumetanide benzyltrimethylammonium dithioacid salt and bumetanide cetyltrimethylammonium dithioacid salt.

24. The prodrug of claim 19, wherein the compound is selected from the group consisting of furosemide, furosemide methyl ester, furosemide cyanomethyl ester, furosemide ethyl ester, furosemide isoamyl ester, furosemide octyl ester, furosemide benzyl ester, furosemide morpholinoethyl ester, furosemide 3-(dimethylaminopropyl) ester, furosemide N,N-diethylglycolamido ester, furosemide N,N-dimethylglycolamido ester, furosemide pivaxetil ester, furosemide propaxetil ester, furosemide methoxy(polyethyleneoxy)ₙ₋₁-ethyl ester, furosemide benzyltrimethylammonium acid salt and furosemide cetyltrimethylammonium acid salt.

25. The prodrug of claim 19, wherein the compound is selected from the group consisting of furosemide S-thioacid, furosemide S-methyl thioester, furosemide S-cyanomethyl thioester, furosemide S-ethyl thioester, furosemide S-isoamyl thioester, furosemide S-octyl thioester, furosemide S-benzyl thioester, furosemide S-(morpholinoethyl) thioester, furosemide S-[3-(dimethylaminopropyl)]thioester, furosemide S-(N,N-diethylglycolamido) thioester, furosemide S-(N,N-dimethylglycolamido) thioester, furosemide S-pivaxetil thioester, furosemide S-propaxetil thioester, furosemide S-[methoxypolyethyleneoxy)ₙ₋₁-ethyl]thioester, furosemide S-benzyltrimethylammonium thioacid salt and furosemide S-cetyltrimethylammonium thioacid salt.

26. The prodrug of claim 19, wherein the compound is selected from the group consisting of metastable furosemide thioacid, furosemide O-methyl thioester, furosemide O-cyanomethyl thioester, furosemide O-ethyl thioester, furosemide O-isoamyl thioester, furosemide O-octyl thioester, furosemide O-benzyl thioester, furosemide O-(morpholinoethyl) thioester, furosemide O-[3-(dimethylaminopropyl)]thioester, furosemide O-(N,N-diethylglycolamido) thioester, furosemide O-(N,N-dimethylglycolamido) thioester, furosemide O-pivaxetil thioester, furosemide O-propaxetil thioester, furosemide O-[methoxy(polyethyleneoxy)ₙ₋₁-ethyl]thioester, furosemide O-benzyltrimethylammonium thioacid salt and O-furosemide cetyltrimethylammonium thioacid salt.

27. The prodrug of claim 19, wherein the compound is selected from the group consisting of furosemide thioaldehyde, furosemide dithioacid, furosemide mesityl dithioester, furosemide cyanomethyl dithioester, furosemide ethyl dithioester, furosemide isoamyl dithioester, furosemide octyl dithioester, furosemide benzyl dithioester, furosemide dibenzylthioamide, furosemide diethylthioamide, furosemide morpholinoethyl dithioester, furosemide 3-(dimethylaminopropyl) dithioester, furosemide N,N-diethylglycolamido dithioester, furosemide N,N-dimethylglycolamido dithioester, furosemide pivaxetil dithioester, furosemide propaxetil dithioester, furosemide methoxy(polyethyleneoxy)ₙ₋₁-ethyl dithioester, furosemide benzyltrimethylammonium dithioacid salt and furosemide cetyltrimethylammonium dithioacid salt.

28. The prodrug of claim 19, wherein the compound is selected from the group consisting of piretanide, piretanide aldehyde, piretanide methyl ester, piretanide cyanomethyl ester, piretanide ethyl ester, piretanide isoamyl ester, piretanide octyl ester, piretanide benzyl ester, piretanide dibenzylamide, piretanide diethylamide, piretanide morpholinoethyl ester, piretanide 3-(dimethylaminopropyl) ester, piretanide N,N-diethylglycolamide ester, piretanide N,N-dimethylglycolamide ester, piretanide pivaxetil ester, piretanide propaxetil ester, piretanide methoxy(polyethyleneoxy)ₙ₋₁-ethyl ester, piretanide benzyltrimethylammonium salt and piretanide cetyltrimethylammonium salt.

29. The prodrug of claim 19, wherein the compound is selected from the group consisting of piretanide thioacid, piretanide S-methyl thioester, piretanide S-cyanomethyl thioester, piretanide S-ethyl thioester, piretanide S-isoamyl thioester, piretanide S-octyl thioester, piretanide S-benzyl thioester, piretanide S-(morpholinoethyl) thioester, piretanide S-[3-(dimethylaminopropyl)]thioester, piretanide S-(N,N-diethylglycolamido) thioester, piretanide S-(N,N-dimethylglycolamido) thioester, piretanide S-pivaxetil thioester, piretanide S-propaxetil thioester, piretanide S-[methoxy(polyethyleneoxy)ₙ₋₁-ethyl]thioester, piretanide S-benzyltrimethylammonium thioacid salt and piretanide S-cetyltrimethylammonium thioacid salt.

30. The prodrug of claim 19, wherein the compound is selected from the group consisting of metastable piretanide thioacid, piretanide O-methyl thioester, piretanide O-cyanomethyl thioester, piretanide O-ethyl thioester, piretanide O-isoamyl thioester, piretanide O-octyl thioester, piretanide O-benzyl thioester, piretanide O-(morpholinoethyl) thioester, piretanide O-[3-(dimethylaminopropyl)]thioester, piretanide O-(N,N-diethylglycolamido) thioester, piretanide, O-(N,N-dimethylglycolamido) thioester, piretanide O-pivaxetil thioester, piretanide O-propaxetil thioester, piretanide O-[methoxy(polyethyleneoxy)ₙ₋₁-ethyl]thioester, piretanide O-benzyltrimethylammonium thioacid salt and piretanide O-cetyltrimethylammonium thioacid salt.

31. The prodrug of claim 19, wherein the compound is selected from the group consisting of piretanide thioaldehyde, piretanide dithioacid, piretanide methyl dithioester, piretanide cyanomethyl dithioester, piretanide ethyl dithioester, piretanide isoamyl dithioester, piretanide octyl dithioester, piretanide benzyl dithioester, piretanide dibenzylthioamide, piretanide diethylthioamide, piretanide morpholinoethyl dithioester, piretanide 3-(dimethylaminopropyl) dithioester, piretanide N,N-diethylglycolamido dithioester, piretanide N,N-dimethylglycolamido dithioester, piretanide pivaxetil dithioester, piretanide propaxetil dithioester, piretanide methoxy(polyethyleneoxy)ₙ₋₁-ethyl dithioester, piretanide benzyltrimethylammonium dithioacid salt and piretanide cetyltrimethylammonium dithioacid salt.

32. The prodrug of claim 19, wherein the compound is present in an amount effective for regulating epilepsy, neuropathic pain, neural function and/or migraines.

33. A prodrug capable of passage across the blood-brain barrier comprising a compound of formula VII: 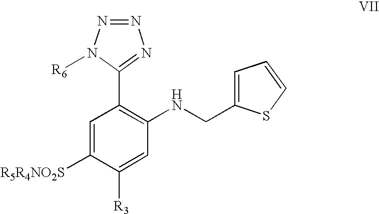
or an ester, pharmaceutically acceptable salt, solvate, tautomer or hydrate thereof wherein
R₃ is selected from the group consisting of aryl, halo, hydroxy, alkoxy, and aryloxy, unsubstituted or substituted;
R₄ and R₅ are each independently selected from the group consisting of hydrogen, alkylaminodialkyl, alkylhydroxyaminodiakyl, unsubstituted or substituted; and
R₆ is selected from the group consisting of alkyloxycarbonylalkyl, alkylaminocarbonyldialkyl, alkylaminodialkyl, alkylhydroxy, a biocompatible polymer, methyloxyalkyl, methyloxyalkaryl, methylthioalkyl and methylthioalkaryl, unsubstituted or substituted.

34. The prodrug of claim 33, wherein the compound is selected from the group consisting of azosemide, tetrazolyl-substituted azosemide, azosemide benzyltrimethylammonium salt, and azosemide cetyltrimethylammonium salt.

35. The prodrug of claim 33 wherein the compound is present in an amount effective for regulating epilepsy, neuropathic pain, neural function and/or migraines.

36. A prodrug capable of passage across the blood-brain barrier comprising a compound of formula VIII: 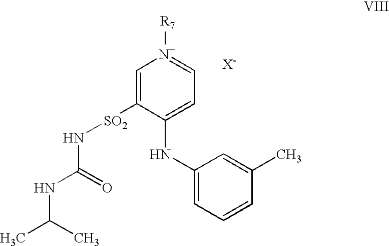
or an ester, pharmaceutically acceptable salt, solvate, tautomer, zwitterion or hydrate thereof, wherein
R₇ is selected from the group consisting of hydrogen, alkyloxycarbonylalkyl, alkylaminocarbonyldialkyl, alkylaminodialkyl, alkylhydroxy, a biocompatible polymer, methyloxyalkyl, methyloxyalkaryl, methylthioalkyl and methylthioalkaryl, unsubstituted or substituted; and
X⁻ is a halide or an anionic moiety; or alternatively, X⁻ is not present.

37. The prodrug of claim 36, wherein the compound is selected from the group consisting of torsemide and a pyridine-substituted torsemide quaternary ammonium salt.

38. The prodrug of claim 36, wherein the compound is present in an amount effective for regulating epilepsy, neuropathic pain, neural function and/or migraines.

39. A pharmaceutical composition comprising a compound selected from the group consisting of the following: 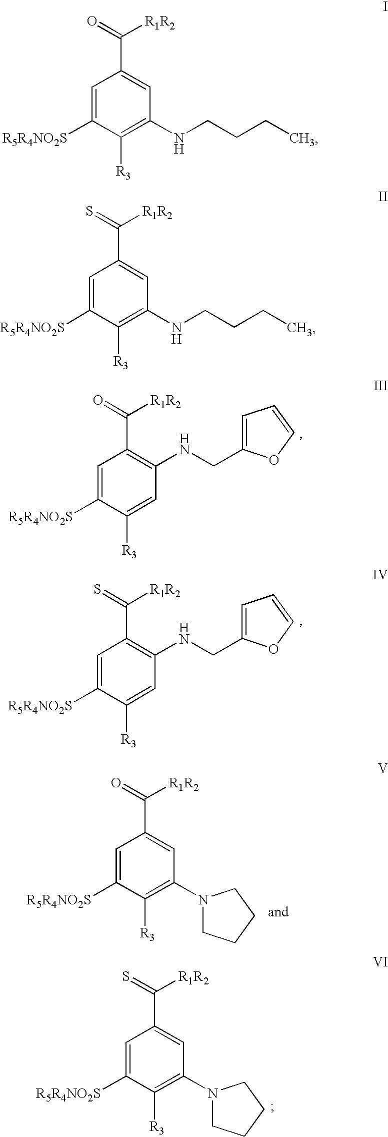
or a pharmaceutically acceptable salt, solvate, tautomer, hydrate or combination thereof,
wherein
R₁ is not present, H, O or S;
R₂ is not present, H or when R₁ is O or S, R₂ is selected from the group consisting of hydrogen, alkyl, aralkyl, aryl, alkylaminodialkyl, alkylcarbonylaminodialkyl, alkyloxycarbonylalkyl, alkylcarbonyloxyalkyl, alkylaldehyde, alkylketoalkyl, alkylamide, alkarylamide, arylamide, an alkylammonium group, alkylcarboxylic acid, alkylheteroaryl, alkylhydroxy, a biocompatible polymer such as alkyloxy(polyalkyloxy)alkylhydroxyl a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, and when R₁ is not present, R₂ is selected from the group consisting of hydrogen, N,N-dialkylamino, N,N-dialkarylamino, N,N-diarylamino, N-alkyl-N-alkarylamino, N-alkyl-N-arylamino, N-alkaryl-N-arylamino, unsubstituted or substituted;
R₃ is selected from the group consisting of aryl, halo, hydroxy, alkoxy, and aryloxy, unsubstituted or substituted; and
R₄ and R₅ are each independently selected from the group consisting of hydrogen, alkylaminodialkyl, carbonylalkyl, carbonylalkaryl, carbonylaryl and salts thereof, with the following provisos:
R₃ of formula I is not phenyloxy when R₁ is O and R₂, R₄ and R₅ are H;
R₃ of formula III is not Cl, when R₁ is O and R₂, R₄ and R₅ are H;
R₂ of formula III is not methyl when R₁ is O, R₃ is Cl, and R₄ and R₅ are H; and
R₃ of formula V is not phenyloxy when R₁ is O and R₂, R₄ and R₅ are H; and
a pharmaceutically acceptable carrier, excipient or diluent.

40. The pharmaceutical composition of claim 39, wherein the compound is present in an amount effective for regulating neuropathic pain, seizures, seizure disorders, epilepsy, status epilepticus, migraine headache, headache, intracranial hypertension, central nervous system edema, neural function, neurotoxicity, head trauma, stroke, ischemia, hypoxia, Alzheimer's Disease, obesity, Parkinson's Disease, neuroprotection and neuronal synchronization.

41. The pharmaceutical composition of claim 39, wherein the compound is selected from the group consisting of bumetanide aldehyde, bumetanide methyl ester, bumetanide cyanomethyl ester, bumetanide ethyl ester, bumetanide isoamyl ester, bumetanide octyl ester, bumetanide benzyl ester, bumetanide dibenzylamide, bumetanide diethylamide, bumetanide morpholinoethyl ester, bumetanide 3-(dimethylaminopropyl) ester, bumetanide N,N-diethylglycolamidoe ester, bumetanide N,N-dimethylglycolamidoe ester, bumetanide pivaxetil ester, bumetanide propaxetil ester, bumetanide methoxy(polyethyleneoxy)ₙ₋₁-ethyl ester, bumetanide benzyltrimethylammonium salt, and bumetanide cetyltrimethylammonium salt.

42. The pharmaceutical composition of claim 39, wherein the compound is selected from the group consisting of bumetanide S-methyl thioester, bumetanide S-cyanomethyl thioester, bumetanide S-ethyl thioester, bumetanide S-isoamyl thioester, bumetanide S-octyl thioester, bumetanide S-benzyl thioester, bumetanide S-(morpholinoethyl) thioester, bumetanide S-[3-(dimethylaminopropyl)]thioester, bumetanide S-(N,N-diethylglycolamido) thioester, bumetanide S-(N,N-dimethylglycolamido) thioester, bumetanide S-pivaxetil thioester, bumetanide S-propaxetil thioester, bumetanide S-methoxy(polyethyleneoxy)ₙ₋₁-ethyl thioester bumetanide S-thioacid (thiobumetanide), bumetanide S-benzyltrimethylammonium thioacid salt, and bumetanide S-cetyltrimethylammonium thioacid salt.

43. The pharmaceutical composition of claim 39, wherein the compound is selected from the group consisting of metastable bumetanide thioacid, bumetanide O-methyl thioester, bumetanide O-cyanomethyl thioester, bumetanide O-ethyl thioester, bumetanide O-isoamyl thioester, bumetanide O-octyl thioester, bumetanide O-benzyl thioester, bumetanide O-(morpholinoethyl) thioester, bumetanide O-[3-(dimethylaminopropyl)]thioester, bumetanide O-(N,N-diethylglycolamido) thioester, bumetanide, O-(N,N-dimethylglycolamido) thioester, bumetanide O-pivaxetil thioester, bumetanide O-propaxetil thioester, bumetanide O-[methoxy(polyethyleneoxy)ₙ₋₁-ethyl thioester, bumetanide O-benzyltrimethylammonium thioacid salt and bumetanide O-cetyltrimethylammonium thioacid salt.

44. The pharmaceutical composition of claim 39, wherein the compound is selected from the group consisting of bumetanide thioaldehyde, bumetanide dithioacid, bumetanide methyl dithioester, bumetanide cyanomethyl dithioester, bumetanide ethyl dithioester, bumetanide isoamyl dithioester, bumetanide octyl dithioester, bumetanide benzyl dithioester, bumetanide dibenzylthioamide bumetanide diethylthioamide, bumetanide morpholinoethyl dithioester, bumetanide 3-(dimethylaminopropyl) dithioester, bumetanide N,N-diethylglycolamido dithioester, bumetanide N,N-dimethylglycolamido dithioester, bumetanide pivaxetil dithioester, bumetanide propaxetil dithioester, bumetanide methoxy(polyethyleneoxy)ₙ₋₁-ethyl dithioester, bumetanide benzyltrimethylammonium dithioacid salt and bumetanide cetyltrimethylammonium dithioacid salt.

45. The pharmaceutical composition of claim 39, wherein the compound is selected from the group consisting of furosemide methyl ester, furosemide cyanomethyl ester, furosemide ethyl ester, furosemide isoamyl ester, furosemide octyl ester, furosemide benzyl ester, furosemide morpholinoethyl ester, furosemide 3-(dimethylaminopropyl) ester, furosemide N,N-diethylglycolamido ester, furosemide N,N-dimethylglycolamido ester, furosemide pivaxetil ester, furosemide propaxetil ester, furosemide methoxypolyethyleneoxy)ₙ₋₁-ethyl ester, furosemide benzyltrimethylammonium acid salt and furosemide cetyltrimethylammonium acid salt.

46. The pharmaceutical composition of claim 39, wherein the compound is selected from the group consisting of furosemide thioacid, furosemide S-methyl thioester, furosemide S-cyanomethyl thioester, furosemide S-ethyl thioester, furosemide S-isoamyl thioester, furosemide S-octyl thioester, furosemide S-benzyl thioester, furosemide S-(morpholinoethyl) thioester, furosemide S-[3-(dimethylaminopropyl)]thioester, furosemide S-(N,N-diethylglycolamido) thioester. furosemide S-(N,N-dimethylglycolamido) thioester, furosemide S-pivaxetil thioester, furosemide S-propaxetil thioester, furosemide S-[methoxy(polyethyleneoxy)ₙ₋₁-ethyl]thioester, furosemide S-benzyltrimethylammonium thioacid salt, and furosemide S-cetyltrimethylammonium thioacid salt.

47. The pharmaceutical composition of claim 39, wherein the compound is selected from the group consisting of metastable furosemide thioacid, furosemide O-methyl thioester, furosemide O-cyanomethyl thioester, furosemide O-ethyl thioester, furosemide O-isoamyl thioester, furosemide O-octyl thioester, furosemide O-benzyl thioester, furosemide O-(morpholinoethyl) thioester, furosemide O-[3-(dimethylaminopropyl)]thioester, furosemide O-(N,N-diethylglycolamido) thioester, furosemide O-(N,N-dimethylglycolamido) thioester, furosemide O-pivaxetil thioester, furosemide O-propaxetil thioester, furosemide O-[methoxy(polyethyleneoxy)ₙ₋₁-ethyl]thioester, furosemide O-benzyltrimethylammonium thioacid salt and furosemide O-cetyltrimethylammonium thioacid salt.

48. The pharmaceutical composition of claim 39, wherein the compound is selected from the group consisting of furosemide thioaldehyde, furosemide dithioacid, furosemide methyl dithioester, furosemide cyanomethyl dithioester, furosemide ethyl dithioester, furosemide isoamyl dithioester, furosemide octyl dithioester, furosemide benzyl dithioester, furosemide dibenzylthioamide, furosemide diethylthioamide, furosemide morpholinoethyl dithioester, furosemide 3-(dimethylaminopropyl) dithioester, furosemide N,N-diethylglycolamido dithioester, furosemide N,N-dimethylglycolamido dithioester, furosemide pivaxetil dithioester, furosemide propaxetil dithioester, furosemide methoxy(polyethyleneoxy)ₙ₋₁-ethyl dithioester, furosemide benzyltrimethylammonium dithioacid salt and furosemide cetyltrimethylammonium dithioacid salt.

49. The pharmaceutical composition of claim 39, wherein the compound is selected from the group consisting of piretanide aldehyde, piretanide methyl ester, piretanide cyanomethyl ester, piretanide ethyl ester, piretanide isoamyl ester, piretanide octyl ester, piretanide benzyl ester, piretanide dibenzylamide, piretanide diethylamide, piretanide morpholinoethyl ester, piretanide 3-(dimethylaminopropyl) ester, piretanide N,N-diethylglycolamide ester, piretanide N,N-dimethylglycolamide ester, piretanide pivaxetil ester, piretanide propaxetil ester, piretanide methoxy(polyethyleneoxy)ₙ₋₁-ethyl ester, piretanide benzyltrimethylammonium salt and piretanide cetyltrimethylammonium salt.

50. The pharmaceutical composition of claim 39, wherein the compound is selected from the group consisting of piretanide thioacid, piretanide S-methyl thioester, piretanide S-cyanomethyl thioester, piretanide S-ethyl thioester, piretanide S-isoamyl thioester, piretanide S-octyl thioester, piretanide S-benzyl thioester, piretanide S-(morpholinoethyl) thioester, piretanide S-[3-(dimethylaminopropyl)]thioester, piretanide S-(N,N-diethylglycolamido) thioester, piretanide S-(N,N-dimethylglycolamido) thioester, piretanide S-pivaxetil thioester, piretanide S-propaxetil thioester, piretanide S-[methoxy(polyethyleneoxy)ₙ₋₁-ethyl]thioester, piretanide S-benzyltrimethylammonium thioacid salt and piretanide S-cetyltrimethylammonium thioacid salt.

51. The pharmaceutical composition of claim 39, wherein the compound is selected from the group consisting of metastable piretanide thioacid, piretanide O-methyl thioester, piretanide O-cyanomethyl thioester, piretanide O-ethyl thioester piretanide O-isoamyl thioester, piretanide O-octyl thioester, piretanide O-benzyl thioester, piretanide O-(morpholinoethyl) thioester, piretanide O-[3-(dimethylaminopropyl)]thioester, piretanide O-(NN-diethylglycolamido) thioester, piretanide, O-(N,N-dimethylglycolamido) thioester, piretanide O-pivaxetil thioester, piretanide O-propaxetil thioester, piretanide O-[methoxy(polyethyleneoxy)ₙ₋₁-ethyl]thioester, piretanide O-benzyltrimethylammonium thioacid salt and piretanide O-cetyltrimethylammonium thioacid salt.

52. The pharmaceutical composition of claim 39, wherein the compound is selected from the group consisting of piretanide thioaldehyde, piretanide dithioacid, piretanide methyl dithioester, piretanide cyanomethyl dithioester, piretanide ethyl dithioester, piretanide isoamyl dithioester, piretanide octyl dithioester, piretanide benzyl dithioester, piretanide dibenzylthioamide, piretanide diethylthioamide, piretanide morpholinoethyl dithioester, piretanide 3-(dimethylaminopropyl) dithioester, piretanide N,N-diethylglycolamido dithioester, piretanide N,N-dimethylglycolamido dithioester, piretanide pivaxetil dithioester, piretanide propaxetil dithioester, piretanide methoxy(polyethyleneoxy)ₙ₋₁-ethyl dithioester, piretanide benzyltrimethylammonium dithioacid salt and piretanide cetyltrimethylammonium dithioacid salt.

53. The pharmaceutical composition of claim 39, wherein the compound is selected from the group consisting of bumetanide methyl ester, bumetanide cyanomethyl ester, bumetanide morpholinoethyl ester, bumetanide 3-(dimethylaminopropyl) ester and bumetanide pivaxetil ester.

54. A pharmaceutical composition comprising a compound of formula VII: 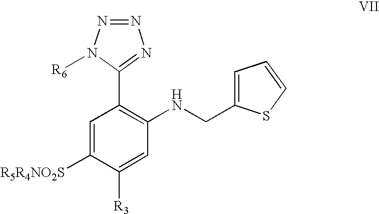
or a pharmaceutically acceptable salt, solvate, tautomer, hydrate or combination thereof, wherein
R₃ is selected from the group consisting of aryl, halo, hydroxy, alkoxy, and aryloxy, unsubstituted or substituted;
R₄ and R₅ are each independently selected from the group consisting of hydrogen, alkylaminodialkyl, alkylhydroxyaminodiakyl, unsubstituted or substituted; and
R₆ is selected from the group consisting of alkyloxycarbonylalkyl, alkylaminocarbonyldialkyl, alkylaminodialkyl, alkylhydroxy, a biocompatible polymer, methyloxyalkyl, methyloxyalkaryl, methylthioalkyl and methylthioalkaryl, unsubstituted or substituted; and
a pharmaceutically acceptable carrier, excipient or diluent.

55. The pharmaceutical composition of claim 54, wherein the compound is present in an amount effective for regulating neuropathic pain, seizures, seizure disorders, epilepsy, status epilepticus, migraine headache, headache, intracranial hypertension, central nervous system edema, neural function, neurotoxicity, head trauma, stroke, ischemia, hypoxia, Alzheimer's Disease, obesity, Parkinson's Disease, neuroprotection and neuronal synchronization.

56. The pharmaceutical composition of claim 54, wherein the compound is a selected from the group consisting of tetrazolyl-substituted azosemide, azosemide benzyltrimethylammonium salt and azosemide cetyltrimethylammonium salt.

57. A pharmaceutical composition comprising a compound of formula VIII: 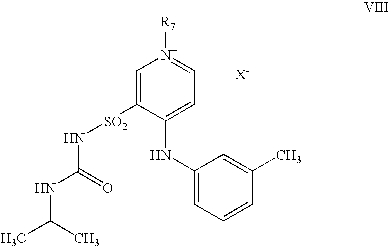
or a pharmaceutically acceptable salt, solvate, tautomer, zwitterion, hydrate or combination thereof, wherein
R₇ is selected from the group consisting of hydrogen, alkyloxycarbonylalkyl, alkylaminocarbonyldialkyl, alkylaminodialkyl, alkylhydroxy, a biocompatible polymer, methyloxyalkyl, methyloxyalkaryl, methylthioalkyl and methylthioalkaryl, unsubstituted or substituted;
X⁻ is a halide or an anionic moiety; or alternatively, X⁻ is not present; and
a pharmaceutically acceptable carrier, excipient or diluent.

58. The pharmaceutical composition of claim 57, wherein the compound is present in an amount effective for regulating neuropathic pain, seizures, seizure disorders, epilepsy, status epilepticus, migraine headache, headache, intracranial hypertension, central nervous system edema, neural function, neurotoxicity, head trauma, stroke, ischemia, hypoxia, Alzheimer's Disease, obesity, Parkinson's Disease, neuroprotection and neuronal synchronization.

59. The pharmaceutical composition of claim 57, wherein the compound is a pyridine-substituted torsemide quaternary ammonium salt.

60. A method of synthesizing a compound of formula I, II, III, IV, V, VI, VII and/or VIII comprising reacting (a) bumetanide or thiobumetanide (bumetanide thioacid), (b) furosemide or thiofurosemide (furosemide thioacid), (c) piretanide or thiopiretanide (piretanide thioacid), (d) azosemide or (e) torsemide with a functional group and/or compound selected from the group consisting of an aluminum hydride, alkyl halide, alcohol, alkaryl halide, mono- and dialkylamine, mono- and dialkarylamine, mono- and diarylamine, and quaternary ammonium hydroxide, unsubstituted or substituted, a biocompatible polymer or combinations thereof, under conditions sufficient to form a compound of formula I, II, III, IV, V, VI, VII and/or VIII.

61. A method of modifying a diuretic or diuretic-like compound to increase lipophilicity of the diuretic or diuretic-like compound comprising reacting the diuretic or diuretic-like compound with a functional croup and/or compound selected from the group consisting of an alkyl halide, alcohol, aldehyde, alkaryl halide, alkyl amine, aryl amine, quaternary ammonium hydroxide and quaternary ammonium salt, unsubstituted or substituted, a biocompatible polymer or combinations thereof, under conditions sufficient to provide a diuretic or diuretic-like compound with increased lipophilic properties compared to an unmodified diuretic or diuretic-like compound.

62. The method of claim 61, wherein the diuretic or diuretic-like compound is selected from the group consisting of bumetanide, thiobumetanide (bumetanide thioacid), furosemide, thiofurosemide (furosemide thioacid), piretanide, thiopiretanide (piretanide thioacid) azosemide, torsemide, indacrinone and oxazolinone.

63. A method of facilitating the passage of a diuretic or diuretic-like compound across the blood-brain barrier comprising reacting the diuretic or diuretic-like compound with a functional group and/or compound selected from the group consisting of an alkyl halide, alcohol, aldehyde, alkaryl halide, mono- and dialkylamine, mono- and dialkarylamine, mono- and diarylamine, quaternary ammonium hydroxide and quaternary ammonium salt, unsubstituted or substituted, a biocompatible polymer or combinations thereof, under conditions sufficient to provide a diuretic or diuretic-like compound capable of passing through the blood-brain barrier.

64. The method of claim 63, wherein the diuretic or diuretic-like compound is selected from the group consisting of bumetanide, furosemide, piretanide, azosemide, torsemide, indacrinone and oxazolinone.

65. A kit comprising one or more containers containing pharmaceutical dosage units comprising an effective amount of one or more compounds of formula I, II, III, IV, V, VI, VII and/or VIII or pharmaceutically acceptable salt, solvate, hydrate, tautomer or combination thereof, wherein the container is packaged with optional instructions for the use thereof.

66. A method of regulating a central nervous system (CNS) disorder comprising administering an effective amount of a compound selected from the group consisting of the following: 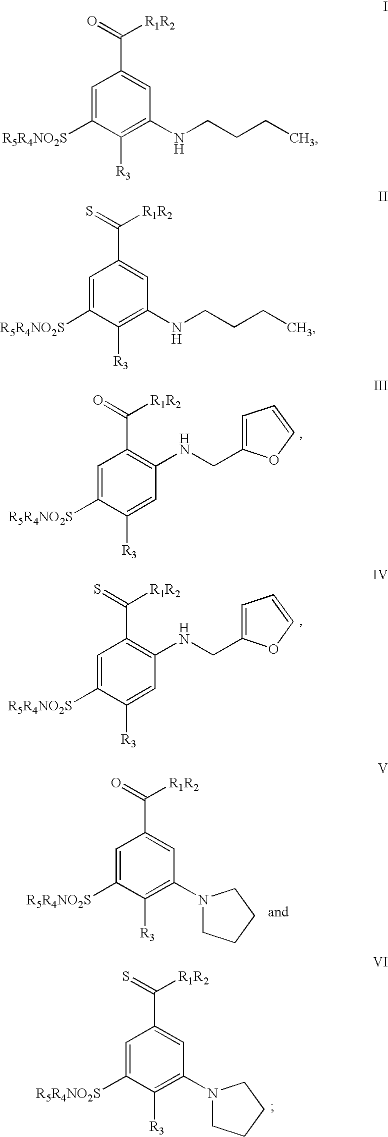
or a pharmaceutically acceptable salt, solvate, tautomer or hydrate thereof, wherein
R₁ is not present, H, O or S;
R₂ is not present, H or when R₁ is O or S, R, is selected from the group consisting of hydrogen, alkyl, aralkyl, aryl, alkylaminodialkyl, alkylcarbonylaminodialkyl, alkyloxycarbonylalkyl, alkylcarbonyloxyalkyl, alkylaldehyde, alkylketoalkyl, alkylamide, alkarylamide, arylamide, an alkylammonium group, alkylcarboxylic acid, alkylheteroaryl, alkylhydroxy, a biocompatible polymer such as alkyloxy(polyalkyloxy)alkylhydroxyl, a polyethylene glycol (PEG), a polyethylene glycol ester (PEG ester) and a polyethylene glycol ether (PEG ether), methyloxyalkyl, methyloxyalkaryl, methylthioalkyl and methylthioalkaryl, unsubstituted or substituted, and when R₁ is not present, R₂ is selected from the group consisting of hydrogen, N,N-dialkylamino, N,N-dialkarylamino, N,N-diarylamino, N-alkyl-N-alkarylamino, N-alkyl-N-arylamino, N-alkaryl-N-arylamino, unsubstituted or substituted;
R₃ is selected from the group consisting of aryl, halo, hydroxy, alkoxy, and aryloxy, unsubstituted or substituted; and
R₄ and R₅ are each independently selected from the group consisting of hydrogen, alkylaminodialkyl, carbonylalkyl, carbonylalkaryl, carbonylaryl and salts thereof.

67. The method of claim 66, wherein the compound is selected from the group consisting of bumetanide methyl ester, bumetanide cyanomethyl ester, bumetanide morpholinoethyl ester, bumetanide 3-(dimethylaminopropyl) ester, bumetanide pivaxetil ester, bumetanide methoxy(polyethyleneoxy)ₙ₋₁-ethyl ester, bumetanide benzyltrimethylammonium acid salt, bumetanide cetyltrimethylammonium acid salt, bumetanide[—(C═O)—SH]thioacid, bumetanide S-methyl thioester, bumetanide S-cyanomethyl thioester, bumetanide S-ethyl thioester, bumetanide S-isoamyl thioester, bumetanide S-octyl thioester, bumetanide S-benzyl thioester, bumetanide S-(morpholinoethyl) thioester, bumetanide S-[3-(dimethylaminopropyl)]thioester, bumetanide S-(N,N-diethylglycolamido) thioester, bumetanide S-(N,N-dimethylglycolamido) thioester, bumetanide S-pivaxetil thioester, bumetanide S-propaxetil thioester, bumetanide S-[methoxy(polyethyleneoxy)ₙ₋₁-ethyl] thioester, bumetanide[—(C═O)—S⁻—]benzyltrimethylammonium thioacid salt and bumetanide[—(C═O)—S⁻]cetyltrimethylammonium thioacid salt, bumetanide[—(C═S)—OH]thioacid, bumetanide O-methyl thioester, bumetanide O-cyanomethyl thioester, bumetanide O-ethyl thioester, bumetanide O-isoamyl thioester, bumetanide O-octyl thioester, bumetanide O-benzyl thioester, bumetanide O-(morpholinoethyl) thioester, bumetanide O-[3-(dimethylaminopropyl)]thioester, bumetanide O-(N,N-diethylglycolamido) thioester, bumetanide, O-(N,N-dimethylglycolamido) thioester, bumetanide O-pivaxetil thioester, bumetanide O-propaxetil thioester, bumetanide O-[methoxy(polyethyleneoxy)ₙ₋₁-ethyl]thioester, bumetanide[—(C═S)—O⁻]benzyltrimethylammonium thioacid salt and bumetanide[—(C═S)—O⁻]cetyltrimethylammonium thioacid salt, bumetanide thioaldehyde, bumetanide[—(C═S)—SH]dithioacid, bumetanide methyl dithioester, bumetanide cyanomethyl dithioester, bumetanide ethyl dithioester, bumetanide isoamyl dithioester, bumetanide octyl dithioester, bumetanide benzyl dithioester, bumetanide dibenzylthioamide, bumetanide diethylthioamide, bumetanide morpholinoethyl dithioester, bumetanide 3-(dimethylaminopropyl) dithioester, bumetanide N,N-diethylglycolamido dithioester, bumetanide N,N-dimethylglycolamido dithioester, bumetanide pivaxetil dithioester, bumetanide propaxetil dithioester, bumetanide methoxy(polyethyleneoxy)ₙ₋₁-ethyl dithioester, bumetanide benzyltrimethylammonium dithioacid salt and bumetanide cetyltrimethylammonium dithioacid salt.

68. The method of claim 66, wherein the compound is selected from the group consisting of furosemide methyl ester, furosemide cyanomethyl ester, bumetanide morpholinoethyl ester, furosemide 3-(dimethylaminopropyl) ester, bumetanide pivaxetil ester, furosemide methoxy(polyethyleneoxy)ₙ₋₁-ethyl ester, furosemide benzyltrimethylammonium acid salt, furosemide cetyltrimethylammonium acid salt, furosemide[—(C═O)—SH]thioacid, furosemide S-methyl thioester, furosemide S-cyanomethyl thioester, furosemide S-ethyl thioester, furosemide S-isoamyl thioester, furosemide S-octyl thioester, furosemide S-benzyl thioester, furosemide S-(morpholinoethyl) thioester, furosemide S-[3-(dimethylaminopropyl)]thioester, furosemide S-(N,N-diethylglycolamido) thioester, furosemide S-(N,N-dimethylglycolamido) thioester, furosemide S-pivaxetil thioester, furosemide S-propaxetil thioester, furosemide S-[methoxy(polyethyleneoxy)ₙ₋₁-ethyl]thioester, furosemide[—(C═O)—S⁻]benzyltrimethylammonium thioacid salt and furosemide[—(C═O)—S⁻]cetyltrimethylammonium thioacid salt, furosemide[—(C═S)—OH]thioacid, furosemide O-methyl thioester, furosemide O-cyanomethyl thioester, furosemide O-ethyl thioester, furosemide O-isoamyl thioester, furosemide O-octyl thioester, furosemide O-benzyl thioester, furosemide O-(morpholinoethyl) thioester, furosemide O-[3-(dimethylaminopropyl)]thioester, furosemide O-(N,N-diethylglycolamido) thioester, furosemide O-N,N-dimethylglycolamido) thioester, furosemide O-pivaxetil thioester, furosemide O-propaxetil thioester, furosemide O-[methoxy(polyethyleneoxy)ₙ₋₁-ethyl]thioester, furosemide[—(C═S)—O⁻] benzyltrimethylammonium thioacid salt and furosemide[—(C═S)—O⁻] cetyltrimethylammonium thioacid salt, furosemide thioaldehyde, furosemide[—(C═S)—SH]dithioacid, furosemide methyl dithioester, bumetanide cyanomethyl dithioester, bumetanide ethyl dithioester, furosemide isoamyl dithioester, furosemide octyl dithioester, furosemide benzyl dithioester, furosemide dibenzylthioamide, furosemide diethylthioamide, furosemide morpholinoethyl dithioester, furosemide 3-(dimethylaminopropyl) dithioester, furosemide N,N-diethylglycolamido dithioester, furosemide N,N-dimethylglycolamido dithioester, furosemide pivaxetil dithioester, furosemide propaxetil dithioester, furosemide methoxy(polyethyleneoxy)ₙ₋₁-ethyl dithioester, furosemide benzyltrimethylammonium dithioacid salt and furosemide cetyltrimethylammonium dithioacid salt.

69. The method of claim 66, wherein the compound is selected from the group consisting of piretanide methyl ester, piretanide cyanomethyl ester, piretanide morpholinoethyl ester, piretanide 3-(dimethylaminopropyl) ester, piretanide pivaxetil ester, piretanide methoxy(polyethyleneoxy)ₙ₋₁-ethyl ester, piretanide benzyltrimethylammonium acid salt, piretanide cetyltrimethylammonium acid salt, piretanide[—(C═O)—SH]thioacid, piretanide S-methyl thioester, piretanide S-cyanomethyl thioester, piretanide S-ethyl thioester, piretanide S-isoamyl thioester, piretanide S-octyl thioester, piretanide S-benzyl thioester, piretanide S-(morpholinoethyl) thioester, piretanide S-[3-(dimethylaminopropyl)]thioester, piretanide S-(N,N-diethylglycolamido) thioester, piretanide S-(N,N-dimethylglycolamido) thioester, piretanide S-pivaxetil thioester, piretanide S-propaxetil thioester, piretanide S-[methoxy(polyethyleneoxy)ₙ₋₁-ethyl]thioester, piretanide[—(C═O)—S⁻]benzyltrimethylammonium thioacid salt, piretanide[—(C═O)—S⁻]cetyltrimethylammonium thioacid salt, piretanide[—(C═S)—OH]thioacid, piretanide O-methyl thioester, piretanide O-cyanomethyl thioester, piretanide O-ethyl thioester, piretanide O-isoamyl thioester, piretanide O-octyl thioester, piretanide O-benzyl thioester, piretanide O-(morpholinoethyl) thioester, piretanide O-[3-(dimethylaminopropyl)]thioester, piretanide O-(N,N-diethylglycolamido) thioester, piretanide, O-(N,N-dimethylglycolamido) thioester, piretanide O-pivaxetil thioester, piretanide O-propaxetil thioester, piretanide O-[methoxy(polyethyleneoxy)ₙ₋₁-ethyl]thioester, piretanide[—(C═S)—O⁻]benzyltrimethylammonium thioacid salt, piretanide[—(C═S)—O⁻]cetyltrimethylammonium thioacid salt, piretanide thioaldehyde, bumetanide[—(C═S)—SH]dithioacid, piretanide methyl dithioester, piretanide cyanomethyl dithioester, piretanide ethyl dithioester, piretanide isoamyl dithioester, piretanide octyl dithioester, piretanide benzyl dithioester, piretanide dibenzylthioamide, piretanide diethylthioamide, bumetanide morpholinoethyl dithioester, bumetanide 3-(dimethylaminopropyl) dithioester, bumetanide N,N-diethylglycolamido dithioester, piretanide N,N-dimethylglycolamido dithioester, piretanide pivaxetil dithioester, piretanide propaxetil dithioester, piretanide methoxy(polyethyleneoxy)ₙ₋₁-ethyl dithioester, piretanide benzyltrimethylammonium dithioacid salt and piretanide cetyltrimethylammonium dithioacid salt.

70. The method of claim 66, wherein the CNS disorder is selected from the group consisting of neuropathic pain, seizures, seizure disorders, epilepsy, status epilepticus, migraine headache, headache, intracranial hypertension, central nervous system edema, neural function, neurotoxicity, head trauma, stroke, ischemia, hypoxia, Alzheimer's Disease, obesity, Parkinson's Disease, neuroprotection and neuronal synchronization.

71. A method of regulating a central nervous system (CNS) disorder comprising administering an effective amount of a compound of formula VII:

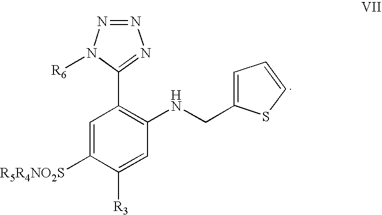

72. A method of regulating a central nervous system (CNS) disorder comprising administering an effective amount of a compound of formula VIII:

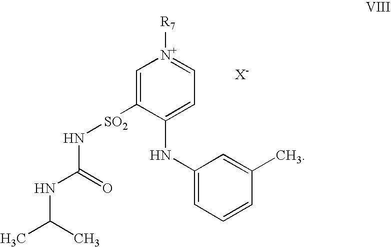

73. A method of regulating a central nervous system (CNS) disorder comprising administering an effective amount of a prodrug of claim 19.

74. A method of regulating a central nervous system (CNS) disorder comprising administering an effective amount of a prodrug of claim 33.

75. A method of regulating a central nervous system (CNS) disorder comprising administering an effective amount of a prodrug of claim 36.

76. A method of regulating epilepsy comprising administering an effective amount of a modified diuretic or diuretic-like compound, wherein said modified diuretic or diuretic-like compound traverses the blood brain barrier.

77. A method of regulating epilepsy comprising administering an effective amount of a compound selected from the group consisting of bumetanide methyl esters bumetanide cyanomethyl ester, bumetanide morpholinoethyl ester, bumetanide 3-(dimethylaminopropyl) ester, bumetanide pivaxetil ester, furosemide methyl ester, furosemide cyanomethyl ester, furosemide morpholinoethyl ester, furosemide 3-(dimethylaminopropyl) ester, furosemide pivaxetil ester, piretanide methyl ester, piretanide cyanomethyl ester, piretanide morpholinoethyl ester, piretanide 3-(dimethylaminopropyl) ester and piretanide pivaxetil ester.

78. A method of regulating epilepsy comprising administering an effective amount of a compound selected from the group consisting of bumetanide S-methyl thioester, bumetanide S-cyanomethyl thioester, bumetanide S-(morpholinoethyl) thioester, bumetanide S-[3-(dimethylaminopropyl)]thioester, bumetanide S-pivaxetil thioester, furosemide S-methyl thioester, furosemide S-cyanomethyl thioester, furosemide S-(morpholinoethyl) thioester, furosemide S-[3-(dimethylaminopropyl)]thioester, furosemide S-pivaxetil thioester, piretanide S-methyl thioester, piretanide S-cyanomethyl thioester, piretanide S-(morpholinoethyl) thioester, piretanide S-[3-(dimethylaminopropyl)]thioester and piretanide S-pivaxetil thioester.

79. A method of regulating epilepsy comprising administering an effective amount of a compound selected from the group consisting of bumetanide O-methyl thioester, bumetanide O-cyanomethyl thioester, bumetanide O-(morpholinoethyl) thioester, bumetanide O-[3-(dimethylaminopropyl)]thioester, bumetanide O-pivaxetil thioester, furosemide O-methyl thioester, furosemide O-cyanomethyl thioester, furosemide O-(morpholinoethyl) thioester, furosemide O-[3-(dimethylaminopropyl)]thioester, furosemide O-pivaxetil thioester, piretanide O-methyl thioester, piretanide O-cyanomethyl thioester, piretanide O-(morpholinoethyl) thioester, piretanide O-[3-(dimethylaminopropyl)]thioester and piretanide O-pivaxetil thioester.

80. A method of regulating epilepsy comprising administering an effective amount of a compound selected from the group consisting of bumetanide methyl dithioester, bumetanide cyanomethyl dithioester, bumetanide morpholinoethyl dithioester, bumetanide 3-(dimethylaminopropyl) dithioester, bumetanide pivaxetil dithioester, furosemide methyl dithioester, furosemide cyanomethyl dithioester, furosemide morpholinoethyl dithioester, furosemide 3-(dimethylaminopropyl) dithioester, furosemide pivaxetil dithioester, piretanide methyl dithioester, piretanide cyanomethyl dithioester, piretanide morpholinoethyl dithioester, piretanide 3-(dimethylaminopropyl) dithioester and piretanide pivaxetil dithioester.

81. A method of regulating epilepsy comprising administering an effective amount of a compound selected from the group consisting of S-bumetanide thioacid, O-bumetanide thioacid, bumetanide dithioacid, S-furosemide thioacid, O-furosemide thioacid, furosemide dithioacid, S-piretanide thioacid, O-piretanide thioacid and piretanide dithioacid.
